(19) United States
(12) Patent Application Publication
(10) Pub. No.: US 2013/0172384 A1
(43) Pub. Date: Jul. 4, 2013

(54) FATTY ACID SYNTHASE INHIBITORS

(71) Applicants: Nicholas D. Adams, Collegeville, PA (US); Christopher Joseph Aquino, Research Triangle Park, NC (US); Jonathan M. Ghergurovich, Collegeville, PA (US); David Lee Musso, Raleigh, NC (US); Cynthia A. Parrish, Collegeville, PA (US)

(72) Inventors: Nicholas D. Adams, Collegeville, PA (US); Christopher Joseph Aquino, Research Triangle Park, NC (US); Jonathan M. Ghergurovich, Collegeville, PA (US); David Lee Musso, Raleigh, NC (US); Cynthia A. Parrish, Collegeville, PA (US)

(73) Assignee: GlaxoSmithKline Intellectual Property Development Limited

(21) Appl. No.: 13/824,000

(22) PCT Filed: Sep. 15, 2011
(86) PCT No.: PCT/US11/51672

§ 371 Date: Mar. 15, 2013

Related U.S. Application Data
(60) Provisional application No. 61/383,774, filed on Sep. 17, 2010.

(51) Int. Cl. C07D 471/04; C07D 413/14; C07D 405/14; C07D 401/14; C07D 403/06

(52) U.S. Cl. CPC C07D 471/04; C07D 401/14; C07D 403/06; C07D 405/14; C07D 413/14
USPC 514/307; 514/300; 514/312; 514/364; 514/384; 546/121; 546/146; 546/158; 548/143; 548/263.2

(57) ABSTRACT

This invention relates to the use of triazolone and triazolethione derivatives for the modulation, notably the inhibition of the activity or function of fatty acid synthase (FAS). Suitably, the present invention relates to the use of triazolones and triazolethiones in the treatment of cancer.

### FIELD OF INVENTION

This invention relates to novel triazolones and triazolethiones which are inhibitors of fatty acid synthase (FAS), to pharmaceutical compositions containing them, to processes for their preparation, and to their use in therapy for the treatment of cancers.

### BACKGROUND

Fatty acids have an essential role in a variety of cellular processes including building blocks for membranes, anchors for targeting membrane proteins, precursors in the synthesis of lipid second messengers and as a medium to store energy, Menendez J S and Lupu R, Fatty acid synthase and the lipogenic phenotype in cancer pathogenesis, Nature Reviews Cancer, 7: 763-777 (2007). Fatty acids can either be obtained from the diet or can be synthesized de novo from carbohydrate precursors. The biosynthesis of the latter is catalyzed by the muliti-functional homodimeric FAS. FAS synthesizes long chain fatty acids by using acetyl-CoA as a primer and Malonyl Co-A as a 2 carbon donor, and NADPH as reducing equivalents (Wakil S J, Lipids, Structure and function of animal fatty acid synthase, 39: 1045-1053 (2004), Asturias F J et al., Structure and molecular organization of mammalian fatty acid synthase, Nature Struct. Mol. Biol. 12:225-232 (2005), Maier T, et al., Architecture of Mammalian Fatty Acid Synthase at 4.5 Å Resolution, Science 311:1258-1262 (2006)).

De novo fatty acid synthesis is active during embryogenesis and in fetal lungs where fatty acids are used for the production of lung surfactant. In adults, most normal human tissues preferentially acquire fatty acids from the diet. Therefore the level of de novo lipogensis and expression of liopogenic enzymes is low (Weiss L, et al., Fatty-acid biosynthesis in man, a pathway of minor importance. Purification, optimal assay conditions, and organ distribution of fatty-acid synthase. Biological Chemistry Hoppe-Seyler 367(9):905-912 (1986)). In contrast, many tumors have high rates of de novo fatty acid synthesis (Medes G, et al., Metabolism of Neoplastic Tissue. IV. A Study of Lipid Synthesis in Neoplastic Tissue Slices in Vitro, Can Res, 13:27-29, (1953)). FAS has now been shown to be overexpressed in numerous cancer types including prostate, ovary, colon, endometrium lung, bladder, stomach and kidney (Kuhajda F P, Fatty-acid synthase and human cancer: new perspectives on its role in tumor biology, Nutrition; 16:202-208 (2000)). This differential expression and function of FAS in tumors and normal cells provide an approach for cancer therapy with the potential of a substantial therapeutic window.

Pharmacological and small interference RNA mediated inhibition of FAS has demonstrated a preferential inhibition of cancer cell proliferation. Additionally these inhibitors induce apoptosis in cancers cells in vitro and retard growth in human tumors in murine xenograft models in vivo (Menendez J S and Lupu R, Nature Reviews Cancer, 7: 763-777 (2007)). Based upon these findings, FAS is considered a major potential target of antineoplastic intervention.

### SUMMARY OF THE INVENTION

This invention relates to compounds of the Formula (I), as shown below:

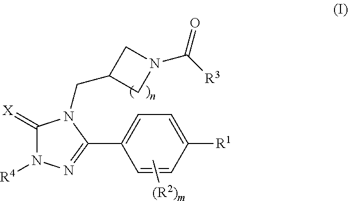

wherein:

R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;

R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;

R⁵ is selected from the group consisting of hydrogen, (C₁-C₄)alkyl, (C₃-C₇)cycloalkyl, phenyl, and phenyl(C₁-C₃)alkyl-;

R⁶ is hydrogen, (C₁-C₄)alkyl, or (C₃-C₇)cycloalkyl;

or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;

R⁷ and R⁸ are each independently hydrogen, (C₁-C₄)alkyl, or (C₃-C₇)cycloalkyl;

or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;

R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶;

X is O or S;

m is 0-3; and

n is 1 or 2;

or pharmaceutically acceptable salts thereof.

This invention also relates to pharmaceutical compositions, which comprise compounds of Formula (I) and pharmaceutically acceptable carriers.

This invention also relates to methods of treating cancer which comprise administering an effective amount of a compound of Formula (I) to a human in need thereof.

This invention also relates to methods of treating cancer which comprise co-administering an compound of Formula (I) and a second compound to a human in need thereof.

### DETAILED DESCRIPTION OF THE INVENTION

This invention relates to compounds of Formula (I), and pharmaceutically acceptable salts thereof.

This invention also relates to compounds of Formula (I)(A):

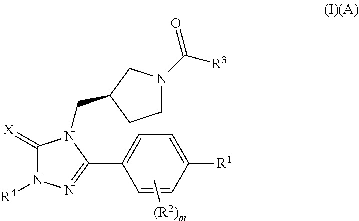

or pharmaceutically acceptable salts thereof, wherein R¹, R², R³, X, and m are defined according to Formula (I).

This invention also relates to compounds of Formula (I)(B):

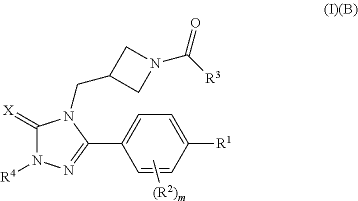

or pharmaceutically acceptable salts thereof, wherein R¹, R², R³, X, and m are defined according to Formula (I).

In one embodiment, this invention relates to compounds of Formula (I), (I)(A), or (I)(B), wherein R¹ is phenyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is 4-fluorophenyl, 4-chlorophenyl, 2,4-difluorophenyl, 2-chloro-4-fluorophenyl, 4-chloro-2-fluorophenyl, 2,4-dichlorophenyl, 4-chloro-2,6-difluorophenyl, 2-fluoro-4-methoxyphenyl, 2-chloro-4-methoxyphenyl, 2,5-difluoro-4-methoxyphenyl, 3-acetylphenyl, 3-(aminocarbonyl)phenyl, 4-(aminocarbonyl)phenyl, 3-cyanophenyl, 4-cyanophenyl, 4-cyano-3-fluorophenyl, 3-(aminosulfonyl)phenyl, 3-methoxyphenyl, 4-methoxyphenyl, 4-trifluoromethoxyphenyl, 4-methoxy-3-methylphenyl, 3-hydroxy-4-methoxyphenyl, 3,4-dimethoxyphenyl, 3-(acetylamino)phenyl, 3-[(aminocarbonyl)amino]phenyl, 3-{[(methylamino)carbonyl]amino}phenyl, 3-{[(n-butylamino)carbonyl]amino}phenyl, 3-{[(dimethylamino)carbonyl]amino}phenyl, 3-[(methylsulfonyl)amino]phenyl, 3-{[(methylamino)sulfonyl]amino}phenyl, 3-{[(dimethylamino)sulfonyl]amino}phenyl, or 3-(5-methyl-1,3,4-oxadiazol-2-yl)phenyl, or pharmaceutically acceptable salts thereof.

In another embodiment, this invention also relates to compounds of Formula (I), (I)(A), or (I)(B), wherein R¹ is 5- or 6-membered heteroaryl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is 2-fluoropyridin-5-yl, or pharmaceutically acceptable salts thereof.

In another embodiment, this invention also relates to compounds of Formula (I), (I)(A), or (I)(B), wherein R¹ is 9- or 10-membered heterocyclyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is benzofuranyl, 2,3-dihydrobenzofuryl, indolyl, indolinyl, benzthiazolyl, benzimidazolyl, benzoxazolyl, indazolyl, pyrrolopyridinyl, imidazopyridinyl, quinolinyl, or isoquinolinyl, wherein said benzofuranyl, 2,3-dihydrobenzofuryl, indolyl, indolinyl, benzthiazolyl, benzimidazolyl, benzoxazolyl, indazolyl, pyrrolopyridinyl, imidazopyridinyl, quinolinyl, or isoquinolinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is benzofuranyl, 2,3-dihydrobenzofuryl, indolyl, benzimidazolyl, benzoxazolyl, indazolyl, imidazopyridinyl, quinolinyl, or isoquinolinyl, wherein said benzofuranyl, 2,3-dihydrobenzofuryl, indolyl, benzimidazolyl, benzoxazolyl, indazolyl, imidazopyridinyl, quinolinyl, or isoquinolinyl is optionally substituted by (C₁-C₄)alkyl, —CF₃, cyano, hydroxyl, methoxy, —OCF₃, amino, methylamino or dimethylamino, or pharmaceutically acceptable salts thereof.

In another embodiment, this invention also relates to compounds of Formula (I), (I)(A), or (I)(B), wherein R¹ is benzofuran-5-yl, 3-methyl-benzofuran-5-yl, 2-carboxy-3-methyl-benzofuran-5-yl, 2,3-dihydro-1-benzofuran-5-yl, benzoxazol-5-yl, 1H-indol-5-yl, 1H-indol-6-yl, 3-cyano-1H-indol-5-yl, 1H-indazol-5-yl, 1H-indazol-6-yl, 3-amino-1H-indazol-6-yl, 1H-benzimidazol-5-yl, imidazo[1,2-a]pyridin-2-yl, imidazo[1,2-a]pyridin-6-yl, 7-methylimidazo[1,2-a]pyridin-6-yl, imidazo[1,2-a]pyridin-7-yl, quinolin-3-yl, quinolin-5-yl, 8-methylquinolin-5-yl, quinolin-6-yl, quinolin-7-yl, or isoquinolin-8-yl, or pharmaceutically acceptable salts thereof.

In another embodiment, this invention also relates to compounds of any of the above embodiments, wherein R² is fluoro, chloro, hydroxyl, methoxy, or methyl, and m is 1, or pharmaceutically acceptable salts thereof.

In another embodiment, this invention also relates to compounds of any of the above embodiments, wherein R³ is (C₁-C₄)alkyl, —CF₃, (C₃-C₆)cycloalkyl, methoxy, or dimethylamino, wherein said (C₃-C₆)cycloalkyl is optionally substituted 1 or 2 times independently by fluoro or methyl, or pharmaceutically acceptable salts thereof.

In another embodiment, this invention also relates to compounds of any of the above embodiments, wherein R³ is methyl, ethyl, isopropyl, t-butyl, —CF₃, cyclopropyl, 1-methyl-cyclopropyl, 2,2-difluoro-cyclopropyl, cyclopentyl, methoxy, or dimethylamino, or pharmaceutically acceptable salts thereof.

In another embodiment, this invention also relates to compounds of any of the above embodiments, wherein R³ is cyclopropyl, or pharmaceutically acceptable salts thereof.

In another embodiment, this invention also relates to compounds of any of the above embodiments, wherein R⁴ is hydrogen, (C₁-C₄)alkyl, —CO(C₁-C₄)alkyl, —CO(C₃-C₆)cycloalkyl, or —CONR⁷R⁸; wherein said (C₁-C₄)alkyl is optionally substituted by phenyl, —CONR⁷R⁸, hydroxyl, (C₁-C₄)alkoxy, or —NR⁷R⁸, or pharmaceutically acceptable salts thereof.

In another embodiment, this invention also relates to compounds of any of the above embodiments, wherein R⁴ is hydrogen, methyl, benzyl, 2-hydroxy-2-methylpropyl, 2-(1-pyrrolidinyl)ethyl, 2-oxo-2-(1-pyrrolidinyl)ethyl, acetyl, cyclopropylcarbonyl, or ethylaminocarbonyl, or pharmaceutically acceptable salts thereof.

In another embodiment, this invention also relates to compounds of any of the above embodiments, wherein X is O, or pharmaceutically acceptable salts thereof.

One particular embodiment of the invention is a compound of Formula (I) wherein:

R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl;

R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;

R⁵ is selected from the group consisting of hydrogen, (C₁-C₄)alkyl, phenyl, and phenyl(C₁-C₃)alkyl-;

R⁶ is hydrogen or (C₁-C₄)alkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl;

R⁷ and R⁸ are each independently hydrogen or (C₁-C₄)alkyl;

or R⁷ and R⁸ taken together with the nitrogen to which they are attached represent a 3- to 7-membered saturated ring optionally containing one other heteroatom which is oxygen, nitrogen, or sulfur;

R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶;

X is O or S;

m is 0-3; and

n is 1 or 2;

or pharmaceutically acceptable salts thereof.

Another particular embodiment of the invention is a compound of Formula (I)(A) wherein:

R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl;

R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;

R⁵ is selected from the group consisting of hydrogen, (C₁-C₄)alkyl, phenyl, and phenyl(C₁-C₃)alkyl-;

R⁶ is hydrogen or (C₁-C₄)alkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl;

R⁷ and R⁸ are each independently hydrogen or (C₁-C₄)alkyl;

or R⁷ and R⁸ taken together with the nitrogen to which they are attached represent a 3- to 7-membered saturated ring optionally containing one other heteroatom which is oxygen, nitrogen, or sulfur;

R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶;

X is O or S; and

m is 0-3;

or pharmaceutically acceptable salts thereof.

Another particular embodiment of the invention is a compound of Formula (I)(B) wherein:

R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl;

R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;

R⁵ is selected from the group consisting of hydrogen, (C₁-C₄)alkyl, phenyl, and phenyl(C₁-C₃)alkyl-;

R⁶ is hydrogen or (C₁-C₄)alkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl;

R⁷ and R⁸ are each independently hydrogen or (C₁-C₄)alkyl;

or R⁷ and R⁸ taken together with the nitrogen to which they are attached represent a 3- to 7-membered saturated ring optionally containing one other heteroatom which is oxygen, nitrogen, or sulfur;

R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶;

X is O or S; and

m is 0-3;

or pharmaceutically acceptable salts thereof.

This invention also relates to the following compounds:
5-[4-(1-benzofuran-5-yl)-3-fluorophenyl]-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)-3-fluorophenyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-[3-fluoro-4-(1H-indol-5-yl)phenyl]-2,4-dihydro-3H-1,2,4-triazol-3-one;
4-{[(3R)-1-(cyclopropylcarbonyl)-3-pyrrolidinyl]methyl}-5-[3-fluoro-4-(1H-indol-6-yl)phenyl]-2,4-dihydro-3H-1,2,4-triazol-3-one;
5-[4-(1-benzofuran-5-yl)-2-fluorophenyl]-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)-2-fluorophenyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-[2-fluoro-4-(1H-indol-5-yl)phenyl]-2,4-dihydro-3H-1,2,4-triazol-3-one;
4-{[(3R)-1-(cyclopropylcarbonyl)-3-pyrrolidinyl]methyl}-5-[2-fluoro-4-(1H-indol-6-yl)phenyl]-2,4-dihydro-3H-1,2,4-triazol-3-one;
N′-[4′-(4-{[(3R)-1-(cyclopropylcarbonyl)-3-pyrrolidinyl]methyl}-5-oxo-4,5-dihydro-1H-1,2,4-triazol-3-yl)-3′-fluoro-3-biphenylyl]-N,N-dimethylsulfamide;
N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3′-fluoro-3-biphenylyl]-N,N-dimethylsulfamide;
4-{[(3R)-1-(cyclopropylcarbonyl)-3-pyrrolidinyl]methyl}-2-methyl-5-[2′,3,5′-trifluoro-4′-(methyloxy)-4-biphenylyl]-2,4-dihydro-3H-1,2,4-triazol-3-one;
5-[4-(1-benzofuran-5-yl)-2-methylphenyl]-4-{[(3R)-1-(cyclopropylcarbonyl)-3-pyrrolidinyl]methyl}-2,4-dihydro-3H-1,2,4-triazol-3-one;
N′-[4′-(4-{[(3R)-1-(cyclopropylcarbonyl)-3-pyrrolidinyl]methyl}-5-oxo-4,5-dihydro-1H-1,2,4-triazol-3-yl)-3′-methyl-3-biphenylyl]-N,N-dimethylsulfamide;
4-{[(3R)-1-(cyclopropylcarbonyl)-3-pyrrolidinyl]methyl}-5-[4-(1H-indol-6-yl)-2-methylphenyl]-2,4-dihydro-3H-1,2,4-triazol-3-one;
4-{[(3R)-1-(cyclopropylcarbonyl)-3-pyrrolidinyl]methyl}-5-(4′-fluoro-3-methyl-4-biphenylyl)-2,4-dihydro-3H-1,2,4-triazol-3-one;
4-{[(3R)-1-(cyclopropylcarbonyl)-3-pyrrolidinyl]methyl}-5-[4-(1H-indol-5-yl)-2-methylphenyl]-2,4-dihydro-3H-1,2,4-triazol-3-one;
5-[2-chloro-4-(1H-indol-6-yl)phenyl]-4-{[(3R)-1-(cyclopropylcarbonyl)-3-pyrrolidinyl]methyl}-2,4-dihydro-3H-1,2,4-triazol-3-one;
5-[2-chloro-4-(1H-indol-5-yl)phenyl]-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)-2-chlorophenyl]-4-{[(3R)-1-(cyclopropylcarbonyl)-3-pyrrolidinyl]methyl}-2,4-dihydro-3H-1,2,4-triazol-3-one;
5-(3-chloro-4′-fluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(1H-indol-6-yl)phenyl]-2,4-dihydro-3H-1,2,4-triazol-3-one;
5-[4-(1-benzofuran-5-yl)phenyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(1H-indazol-6-yl)phenyl]-2,4-dihydro-3H-1,2,4-triazol-3-one;
4-{[(3R)-1-(cyclopropylcarbonyl)-3-pyrrolidinyl]methyl}-5-[4-(1H-indazol-5-yl)phenyl]-2,4-dihydro-3H-1,2,4-triazol-3-one;
5-(4′-chloro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2,4-dihydro-3H-1,2,4-triazol-3-one;
5-[2′-chloro-4′-(methyloxy)-4-biphenylyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(1H-indol-5-yl)phenyl]-2,4-dihydro-3H-1,2,4-triazol-3-one;
5-[4-(1H-benzimidazol-5-yl)phenyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-(2′,4′-dichloro-4-biphenylyl)-2,4-dihydro-3H-1,2,4-triazol-3-one;
4-{[(3R)-1-(cyclopropylcarbonyl)-3-pyrrolidinyl]methyl}-5-[4-(1H-indol-6-yl)phenyl]-2-methyl-2,4-dihydro-3H-1,2,4-triazol-3-one;
5-[4-(1-benzofuran-5-yl)phenyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-[4-(1H-indol-5-yl)phenyl]-2-methyl-2,4-dihydro-3H-1,2,4-triazol-3-one;
4-{[(3R)-1-(cyclopropylcarbonyl)-3-pyrrolidinyl]methyl}-5-[4-(1H-indazol-6-yl)phenyl]-2-methyl-2,4-dihydro-3H-1,2,4-triazol-3-one;
5-[2′-chloro-4′-(methyloxy)-4-biphenylyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-methyl-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-(2-hydroxy-2-methylpropyl)-2,4-dihydro-3H-1,2,4-triazol-3-one;
2-acetyl-4-{[(3R)-1-(cyclopropylcarbonyl)-3-pyrrolidinyl]methyl}-5-(4′-fluoro-4-biphenylyl)-2,4-dihydro-3H-1,2,4-triazol-3-one;
2-(cyclopropylcarbonyl)-4-{[(3R)-1-(cyclopropylcarbonyl)-3-pyrrolidinyl]methyl}-5-(4′-fluoro-4-biphenylyl)-2,4-dihydro-3H-1,2,4-triazol-3-one;
4-{[(3R)-1-(cyclopropylcarbonyl)-3-pyrrolidinyl]methyl}-N-ethyl-3-(4′-fluoro-4-biphenylyl)-5-oxo-4,5-dihydro-1H-1,2,4-triazole-1-carboxamide;
4-{[(3R)-1-(cyclopropylcarbonyl)-3-pyrrolidinyl]methyl}-5-(4′-fluoro-4-biphenylyl)-2-[2-oxo-2-(1-pyrrolidinyl)ethyl]-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-(phenylmethyl)-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-[2-(1-pyrrolidinyl)ethyl]-2,4-dihydro-3H-1,2,4-triazol-3-one;
4-{[(3R)-1-(cyclopropylcarbonyl)-3-pyrrolidinyl]methyl}-5-(2′,3,4′-trifluoro-4-biphenylyl)-2,4-dihydro-3H-1,2,4-triazol-3-one;
4-{[(3R)-1-(cyclopropylcarbonyl)-3-pyrrolidinyl]methyl}-5-[3-fluoro-4′-(methyloxy)-4-biphenylyl]-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-3′-fluoro-4-biphenylcarbonitrile;
5-(4′-chloro-3-fluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[3′-(methyloxy)-4-biphenylyl]-2,4-dihydro-3H-1,2,4-triazol-3-one;
N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylsulfamide;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]methanesulfonamide;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]acetamide;
4′-(4-{[(3R)-1-(cyclopropylcarbonyl)-3-pyrrolidinyl]methyl}-5-oxo-4,5-dihydro-1H-1,2,4-triazol-3-yl)-3-biphenylsulfonamide;
4′-(4-{[(3R)-1-(cyclopropylcarbonyl)-3-pyrrolidinyl]methyl}-5-oxo-4,5-dihydro-1H-1,2,4-triazol-3-yl)-3-biphenylcarboxamide;
4′-(4-{[(3R)-1-(cyclopropylcarbonyl)-3-pyrrolidinyl]methyl}-5-oxo-4,5-dihydro-1H-1,2,4-triazol-3-yl)-3-biphenylcarbonitrile;
4′-(4-{[(3R)-1-(cyclopropylcarbonyl)-3-pyrrolidinyl]methyl}-5-oxo-4,5-dihydro-1H-1,2,4-triazol-3-yl)-3-fluoro-4-biphenylcarbonitrile;
4-{[(3R)-1-(cyclopropylcarbonyl)-3-pyrrolidinyl]methyl}-5-[4-(7-methylimidazo[1,2-a]pyridin-6-yl)phenyl]-2,4-dihydro-3H-1,2,4-triazol-3-one;
5-(3′-acetyl-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(6-fluoro-3-pyridinyl)phenyl]-2,4-dihydro-3H-1,2,4-triazol-3-one;
4-{[(3R)-1-(cyclopropylcarbonyl)-3-pyrrolidinyl]methyl}-5-(4-imidazo[1,2-a]pyridin-6-ylphenyl)-2,4-dihydro-3H-1,2,4-triazol-3-one;
4-{[(3R)-1-(cyclopropylcarbonyl)-3-pyrrolidinyl]methyl}-5-[3′-(5-methyl-1,3,4-oxadiazol-2-yl)-4-biphenylyl]-2,4-dihydro-3H-1,2,4-triazol-3-one;
5-[3′,4′-bis(methyloxy)-4-biphenylyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(2,3-dihydro-1-benzofuran-5-yl)phenyl]-2,4-dihydro-3H-1,2,4-triazol-3-one;
4-{[(3R)-1-(cyclopropylcarbonyl)-3-pyrrolidinyl]methyl}-5-{4′-[(trifluoromethyl)oxy]-4-biphenylyl}-2,4-dihydro-3H-1,2,4-triazol-3-one;
4-{[(3R)-1-(cyclopropylcarbonyl)-3-pyrrolidinyl]methyl}-5-[2′,5′-difluoro-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-[2′-fluoro-4′-(methyloxy)-4-biphenylyl]-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-4-biphenylcarbonitrile;
4′-(4-{[(3R)-1-(cyclopropylcarbonyl)-3-pyrrolidinyl]methyl}-5-oxo-4,5-dihydro-1H-1,2,4-triazol-3-yl)-4-biphenylcarboxamide;
N-butyl-N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea;
5-(2′-chloro-4′-fluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(3-quinolinyl)phenyl]-2,4-dihydro-3H-1,2,4-triazol-3-one;
4-{[(3R)-1-(cyclopropylcarbonyl)-3-pyrrolidinyl]methyl}-5-[4-(5-quinolinyl)phenyl]-2,4-dihydro-3H-1,2,4-triazol-3-one;
4-{[(3R)-1-(cyclopropylcarbonyl)-3-pyrrolidinyl]methyl}-5-[4-(6-quinolinyl)phenyl]-2,4-dihydro-3H-1,2,4-triazol-3-one;
4-{[(3R)-1-(cyclopropylcarbonyl)-3-pyrrolidinyl]methyl}-5-[4-(7-quinolinyl)phenyl]-2,4-dihydro-3H-1,2,4-triazol-3-one;
4-{[(3R)-1-(cyclopropylcarbonyl)-3-pyrrolidinyl]methyl}-5-[3′-hydroxy-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-[3′-methyl-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-[4-(8-methyl-5-quinolinyl)phenyl]-2,4-dihydro-3H-1,2,4-triazol-3-one;
5-(4′-chloro-2′,6′-difluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-(4-imidazo[1,2-a]pyridin-2-ylphenyl)-2,4-dihydro-3H-1,2,4-triazol-3-one;
4-{[(3R)-1-(cyclopropylcarbonyl)-3-pyrrolidinyl]methyl}-5-(4-imidazo[1,2-a]pyridin-7-ylphenyl)-2,4-dihydro-3H-1,2,4-triazol-3-one;
4-{[(3R)-1-(cyclopropylcarbonyl)-3-pyrrolidinyl]methyl}-5-[4-(8-isoquinolinyl)phenyl]-2,4-dihydro-3H-1,2,4-triazol-3-one;
5-[4-(3-amino-1H-indazol-6-yl)phenyl]-4-{[(3R)-1-(cyclopropylcarbonyl)-3-pyrrolidinyl]methyl}-2,4-dihydro-3H-1,2,4-triazol-3-one;
5-[4-(1,3-benzoxazol-5-yl)phenyl]-4-{[(3R)-1-(cyclopropylcarbonyl)-3-pyrrolidinyl]methyl}-2,4-dihydro-3H-1,2,4-triazol-3-one;
N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3-biphenylyl]-N,N-dimethylsulfamide;
4′-(4-{[(3R)-1-(cyclopropylcarbonyl)-3-pyrrolidinyl]methyl}-1-methyl-5-oxo-4,5-dihydro-1H-1,2,4-triazol-3-yl)-3-fluoro-4-biphenylcarbonitrile;
4-{[(3R)-1-(cyclopropylcarbonyl)-3-pyrrolidinyl]methyl}-5-(4-imidazo[1,2-a]pyridin-6-ylphenyl)-2-methyl-2,4-dihydro-3H-1,2,4-triazol-3-one;
5-[4-(3-amino-1H-indazol-6-yl)phenyl]-4-{[(3R)-1-(cyclopropylcarbonyl)-3-pyrrolidinyl]methyl}-2-methyl-2,4-dihydro-3H-1,2,4-triazol-3-one;
5-[4-(4-{[(3R)-1-(cyclopropylcarbonyl)-3-pyrrolidinyl]methyl}-5-oxo-4,5-dihydro-1H-1,2,4-triazol-3-yl)phenyl]-3-methyl-1-benzofuran-2-carboxylic acid;
4-{[(3R)-1-(cyclopropylcarbonyl)-3-pyrrolidinyl]methyl}-5-[4-(3-methyl-1-benzofuran-5-yl)phenyl]-2,4-dihydro-3H-1,2,4-triazol-3-one;
5-[4-(4-{[(3R)-1-(cyclopropylcarbonyl)-3-pyrrolidinyl]methyl}-5-oxo-4,5-dihydro-1H-1,2,4-triazol-3-yl)phenyl]-1H-indole-3-carbonitrile;
5-[4-(1-benzofuran-5-yl)phenyl]-4-{[(3R)-1-(cyclopentylcarbonyl)-3-pyrrolidinyl]methyl}-2,4-dihydro-3H-1,2,4-triazol-3-one;
5-[4-(1-benzofuran-5-yl)phenyl]-4-{[(3R)-1-(2-methylpropanoyl)-3-pyrrolidinyl]methyl}-2,4-dihydro-3H-1,2,4-triazol-3-one;
(3R)-3-({3-[4-(1-benzofuran-5-yl)phenyl]-5-oxo-1,5-dihydro-4H-1,2,4-triazol-4-yl}methyl)-N,N-dimethyl-1-pyrrolidinecarboxamide;
5-[4-(1-benzofuran-5-yl)phenyl]-4-{[(3R)-1-propanoyl-3-pyrrolidinyl]methyl}-2,4-dihydro-3H-1,2,4-triazol-3-one;
methyl(3S)-3-({3-[4-(1-benzofuran-5-yl)phenyl]-5-oxo-1,5-dihydro-4H-1,2,4-triazol-4-yl}methyl)-1-pyrrolidinecarboxylate;
4-{[(3R)-1-acetyl-3-pyrrolidinyl]methyl}-5-[4-(1-benzofuran-5-yl)phenyl]-2,4-dihydro-3H-1,2,4-triazol-3-one;
5-[4-(1-benzofuran-5-yl)phenyl]-4-{[(3R)-1-(2,2-dimethylpropanoyl)-3-pyrrolidinyl]methyl}-2,4-dihydro-3H-1,2,4-triazol-3-one;
5-[4-(1-benzofuran-5-yl)phenyl]-4-({(3R)-1-[(1-methylcyclopropyl)carbonyl]-3-pyrrolidinyl}methyl)-2,4-dihydro-3H-1,2,4-triazol-3-one;
5-[4-(1-benzofuran-5-yl)phenyl]-4-{[(3R)-1-(trifluoroacetyl)-3-pyrrolidinyl]methyl}-2,4-dihydro-3H-1,2,4-triazol-3-one;
5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer A);
5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer B);
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylurea;
N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylurea;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylsulfamide;
5-(4-bromophenyl)-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)phenyl]-4-{[(3R)-1-(cyclopropylcarbonyl)-3-pyrrolidinyl]methyl}-2,4-dihydro-3H-1,2,4-triazole-3-thione;
5-[2′-chloro-4′-(methyloxy)-4-biphenylyl]-4-{[(3R)-1-(cyclopropylcarbonyl)-3-pyrrolidinyl]methyl}-2,4-dihydro-3H-1,2,4-triazole-3-thione;
4-{[(3R)-1-(cyclopropylcarbonyl)-3-pyrrolidinyl]methyl}-5-[4-(1H-indol-6-yl)phenyl]-2,4-dihydro-3H-1,2,4-triazole-3-thione;
4-{[(3R)-1-(cyclopropylcarbonyl)-3-pyrrolidinyl]methyl}-5-(4′-fluoro-4-biphenylyl)-2,4-dihydro-3H-1,2,4-triazole-3-thione;
4-{[(3R)-1-(cyclopropylcarbonyl)-3-pyrrolidinyl]methyl}-5-(3,4′-difluoro-4-biphenylyl)-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-3,3′-difluoro-4-biphenylcarbonitrile;
4-{[(3R)-1-(cyclopropylcarbonyl)-3-pyrrolidinyl]methyl}-5-[2′,3-difluoro-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-(2′-fluoro-4-biphenylyl)-2,4-dihydro-3H-1,2,4-triazol-3-one;
4-{[(3R)-1-(cyclopropylcarbonyl)-3-pyrrolidinyl]methyl}-5-(2′,3-difluoro-4-biphenylyl)-2,4-dihydro-3H-1,2,4-triazol-3-one;
4-{[(3R)-1-(cyclopropylcarbonyl)-3-pyrrolidinyl]methyl}-5-[2-fluoro-4-(7-quinolinyl)phenyl]-2,4-dihydro-3H-1,2,4-triazol-3-one;
4-{[(3R)-1-(cyclopropylcarbonyl)-3-pyrrolidinyl]methyl}-5-[2-fluoro-4-(3-quinolinyl)phenyl]-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-2,3′-difluoro-4-biphenylcarbonitrile;
4-{[(3R)-1-(cyclopropylcarbonyl)-3-pyrrolidinyl]methyl}-5-[4-(2-hydroxy-3-quinolinyl)phenyl]-2,4-dihydro-3H-1,2,4-triazol-3-one; and
4-{[(3R)-1-(cyclopropylcarbonyl)-3-pyrrolidinyl]methyl}-5-[4′-(methyloxy)-4-biphenylyl]-2,4-dihydro-3H-1,2,4-triazol-3-one-d₂;

and pharmaceutically acceptable salts thereof.

This invention also relates to compounds exemplified in the Experimental section. Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted. It is to be understood that the present invention includes all combinations and subsets of the particular groups defined hereinabove.

The invention also includes various deuterated forms of the compounds of Formula (I). Each available hydrogen atom attached to a carbon atom may be independently replaced with a deuterium atom. A person of ordinary skill in the art will know how to synthesize deuterated forms of the compounds of Formula (I). Commercially available deuterated starting materials may be employed in the preparation of deuterated forms of the compounds of Formula (I), or they may be synthesized using conventional techniques employing deuterated reagents (e.g. lithium aluminum deuteride).

### DEFINITIONS

Terms are used within their accepted meanings. The following definitions are meant to clarify, but not limit, the terms defined.

As used herein, the term “alkyl” refers to a straight or branched chain hydrocarbon radical, preferably having from one to twelve carbon atoms, which may be unsubstituted or substituted, saturated or unsaturated with multiple degrees of substitution included within the present invention. When optionally substituted, the alkyl group is unsubstituted or substituted with suitable substituents selected from the group consisting of halogen, amino, substituted amino, cyano, hydroxyl, alkoxy, alkylthio, alkylsulfonyl, aminosulfonyl, carboxylic acid, carboxylic ester, carboxamide, aminocarbonyl, and heterocyclyl. Examples of “alkyl” as used herein include, but are not limited to, methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —OR^{a}, where R^{a} is (C₁-C₄)alkyl or (C₃-C₇)cycloalkyl as defined above.

As used herein, the term “heterocyclyl” refers to an unsubstituted or substituted mono- or polycyclic ring system containing one or more heteroatoms. Preferred heteroatoms include nitrogen, oxygen, and sulfur, including N-oxides, sulfur oxides, and dioxides. The term “9- or 10-membered heterocyclyl” represents a fully unsaturated or partially unsaturated, bicyclic group, containing 9 or 10 ring atoms, including 1 to 5 heteroatoms independently selected from nitrogen, oxygen and sulfur, which group may be unsubstituted or substituted by one or more of the substituents defined herein. Selected 9- or 10-membered heterocycyl groups contain one nitrogen, oxygen or sulfur ring heteroatom, and optionally contain 1, 2, 3, or 4 additional nitrogen ring atoms and/or 1 additional oxygen or sulfur atom. Examples of 9- or 10-membered heterocyclyl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the term “heteroaryl”, unless otherwise defined, refers to an aromatic ring system containing carbon atom(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 heteroatoms. Bicyclic heteroaryl rings may contain from 8 to 10 member atoms. The 5-membered heteroaryl groups present in the compounds of this invention contain one nitrogen, oxygen, or sulfur ring heteroatom, and optionally contain 1, 2, or 3 additional nitrogen ring atoms. The 6-membered heteroaryl groups present in the compounds of this invention contain 1, 2, 3, or 4 nitrogen ring heteroatoms. Examples of 5- or 6-membered heteroaryl groups include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

As used herein, the term “cyano” refers to the group —CN.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups include acyl, alkyl, alkylsulfonyl, alkoxy, alkoxycarbonyl, cyano, halogen, haloalkyl, hydroxyl, oxo, amide, sulfamide, urea, amino, substituted amino, acylamino, phenylcarbonyl, dialkylaminosulfonamide, morpholino, sulfonamide, thiourea, nitro, pyrrolidinyl, pyrazolyl, pyrrolyl, phenyl, and tetrazolyl, wherein pyrrolidinyl, pyrazolyl and tetrazolyl can be further substituted with one to three (C₁-C₃)alkyl.

The invention further provides a pharmaceutical composition (also referred to as pharmaceutical formulation) comprising a compound of Formula (I) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula (I) or salt thereof with at least one excipient.

### PHARMACEUTICAL COMPOSITIONS

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from disease conditions targeted by the present compounds. Such treatment comprises the step of administering a therapeutically effective amount of a compound of Formula (I) or salt thereof to said mammal, particularly a human. Treatment can also comprise the step of administering a therapeutically effective amount of a pharmaceutical composition containing a compound of Formula (I) or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

While it is possible that, for use in therapy, a therapeutically effective amount of a compound of Formula (I) or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula (I) or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula (I) per se. Similar dosages should be appropriate for treatment (including prophylaxis) of the other conditions referred herein for treatment. In general, determination of appropriate dosing can be readily arrived at by one skilled in medicine or the pharmacy art.

### Combinations

When a compound of Formula (I) is administered for the treatment of cancer, the term “co-administering” and derivatives thereof as used herein is meant either simultaneous administration or any manner of separate sequential administration of a FAS inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracycline, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present FAS inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME@ or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

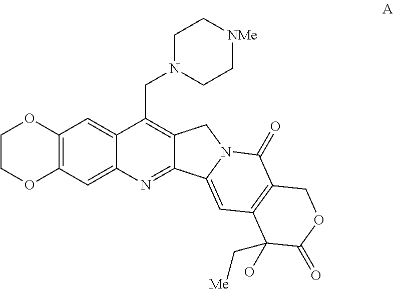 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta) IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and Bennett, C. F. and Cowsert, L. M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alpha beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of Formula (I) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

### EXPERIMENTALS

Abbreviations: aq., aqueous; Boc₂O, di-tert-butyl dicarbonate; CDI, 1,1′-carbonyldiimidazole; CH₂Cl₂, dichloromethane; CHCl₃, chloroform; CH₃CN, acetonitrile; Cs₂CO₃, cesium carbonate; CsF, cesium fluoride; d, day(s); DBU, 1,8-diazabicyclo[5.4.0]undec-7-ene; DIAD, diisopropyl azodicarboxylate; DIPEA, diisopropylethylamine; DMF, N,N-dimethylformamide; DMSO, dimethylsulfoxide; EDC, N-(3-dimethylaminopropyl)-N′-ethylcarbodiimide hydrochloride; Et₂O, diethyl ether; EtOAc, ethyl acetate; EtOH, ethanol; h, hour(s); HCl, hydrochloric acid; H₂O, water; HOAc, acetic acid; HOAt, 1-hydroxy-7-azabenzotriazole; HOBt, 1-hydroxybenzotriazole; K₂CO₃, potassium carbonate; KOAc, potassium acetate; KOCN, potassium cyanate; K₃PO₄, potassium phosphate; MeOH, methanol; MgSO₄, magnesium sulfate; min., minute(s); N₂, nitrogen gas; NaHCO₃, sodium bicarbonate; NaOAc, sodium acetate; Na₂SO₄, sodium sulfate; NH₄OH, ammonium hydroxide; NMP, N-methylpyrrolidone; PdCl₂(dppf), 1,1′-bis(diphenylphosphino)ferrocene-palladium(II)dichloride.dichloromethane complex; Pd(P-t-Bu₃)₂, bis(tri-tert-butylphosphine)palladium(0); Pd(PPh₃)₄, tetrakis(triphenylphosphine)palladium(0); PPh₃, triphenylphosphine; i-PrOH, isopropyl alcohol; THF, tetrahydrofuran; TFA, trifluoroacetic acid.

### Preparation

The derivatives described herein were prepared by the general methods described below:

### SCHEMES/EXPERIMENTALS

The benzohydrazide intermediates can be prepared according to Scheme I and coupled to a pyrrolidine or azetidine carbamate intermediate using a reagent such as CDI. These acylsemicarbazide intermediates can then be cyclized to a triazolone under refluxing aqueous potassium carbonate conditions. The pyrrolidine amine can then be acylated.

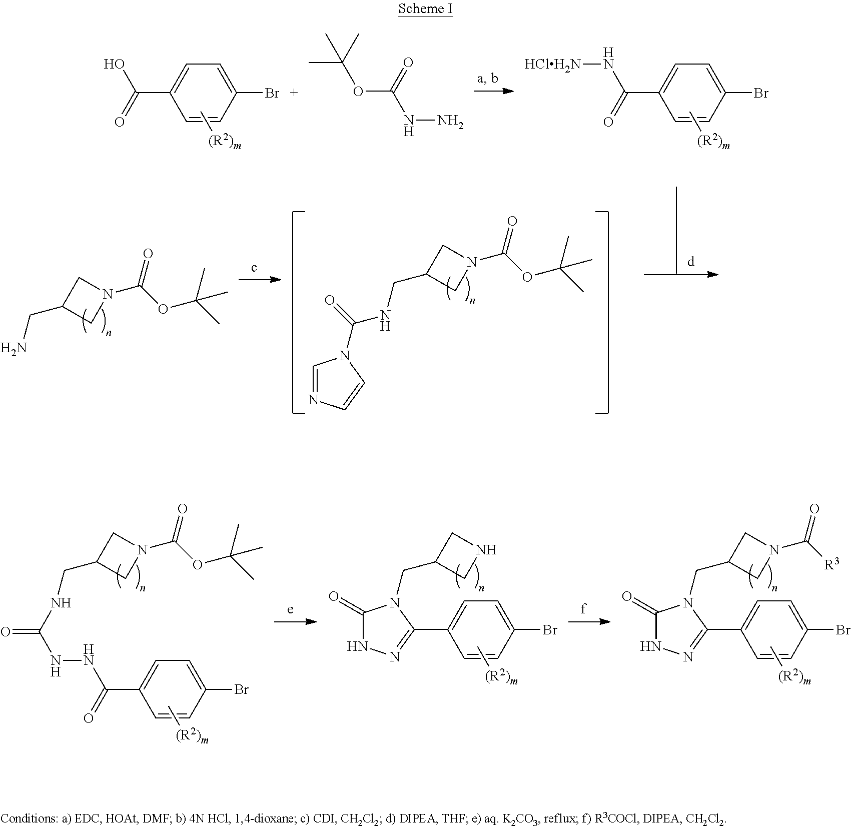

The benzohydrazide intermediates can be coupled to a pyrrolidine or azetidine amide intermediates using a reagent such as CDI. These acylsemicarbazide intermediates can then be cyclized to a triazolone under refluxing aq. potassium carbonate (Scheme II).

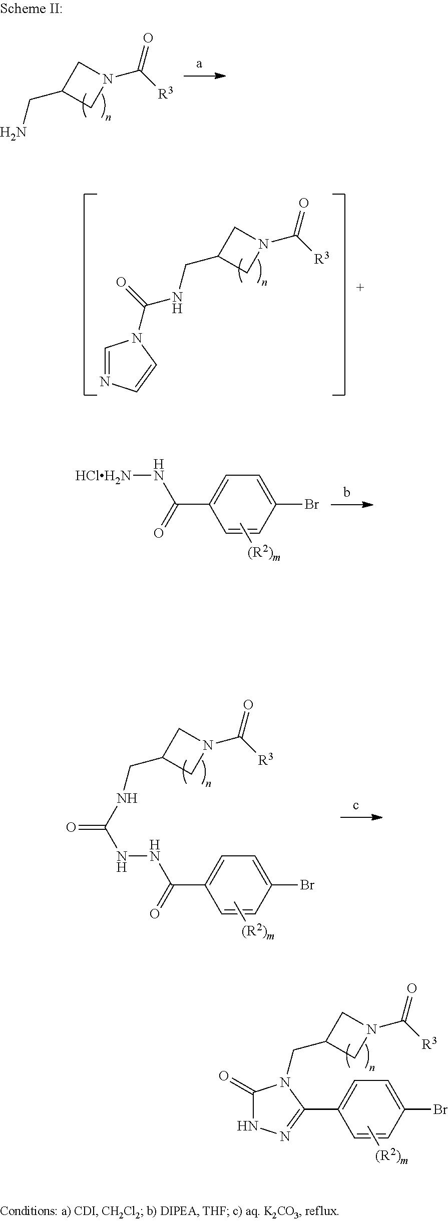

The triazolone phenylbromides can then be coupled with an aryl boronic ester/acid under Suzuki coupling conditions or can first be converted to the intermediate boronic ester to allow metal-mediated cross-coupling with aryl halides or sulfonates (Scheme III).

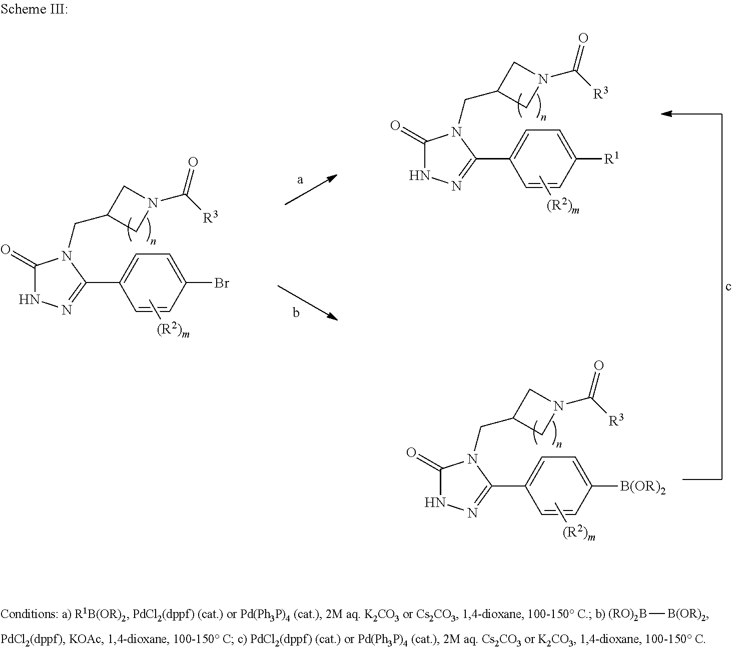

Substitution of the triazolone core can occur at the N-1 nitrogen under conditions shown in Scheme IV.

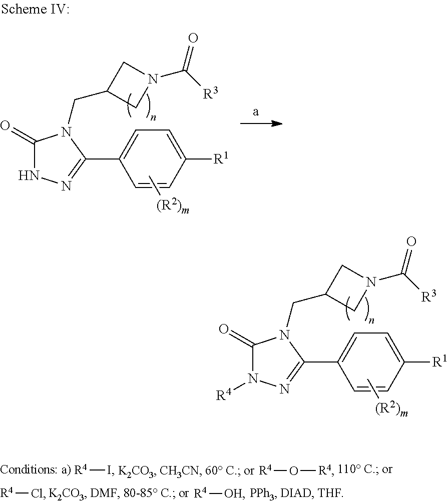

As shown in Scheme V, a similar route can be followed to prepare the corresponding triazolethiones using a reagent such as 1,1′-thiocarbonyldiimidazole. Subsequent elaboration to the final products can occur following similar conditions to those outlined above.

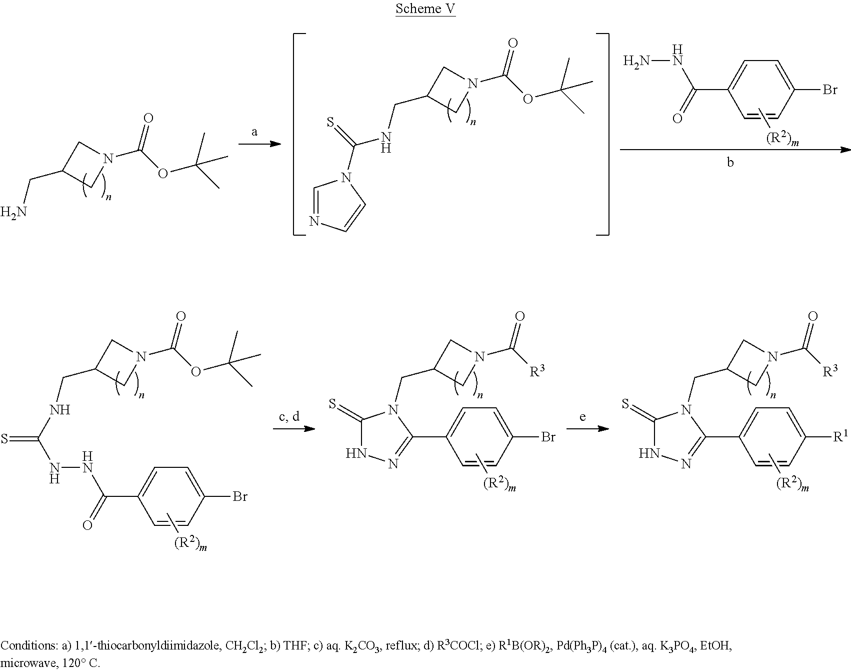

### Example 1

### 5-[4-(1-benzofuran-5-yl)-3-fluorophenyl]-4-{[(3R)-1-(cyclopropylcarbonyl)-3-pyrrolidinyl]methyl}-2,4-dihydro-3H-1,2,4-triazol-3-one

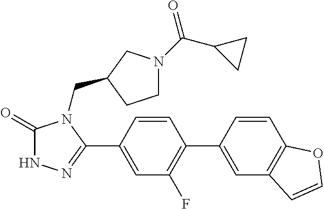 [SMILES: O=C(C1CC1)N1CC[C@@H](Cn2c(-c3ccc(-c4ccc5occc5c4)c(F)c3)n[nH]c2=O)C1]

### a) 1,1-dimethylethyl 2-[(4-bromo-3-fluorophenyl)carbonyl]hydrazinecarboxylate

To a solution of 4-bromo-3-fluorobenzoic acid (2.50 g, 11.42 mmol) and tert-butyl hydrazinecarboxylate (2.06 g, 11.42 mmol) in DMF (50 mL) were added HOAt (0.155 g, 1.142 mmol) followed by EDC (2.63 g, 13.70 mmol). The reaction was stirred at room temperature under nitrogen. After 2 h, the reaction was diluted with water (150 mL), causing a precipitate to form. After stirring for 10 min, the precipitate was collected by filtration, rinsed with water, and dried to constant weight to afford the title compound (3.26 g, 86%) as a white solid. LC-MS: m/e=333 [M+23]⁺.

### b) 4-bromo-3-fluorobenzohydrazide hydrochloride

To a mixture of 1,1-dimethylethyl 2-[(4-bromo-3-fluorophenyl)carbonyl]hydrazinecarboxylate (3.27 g, 9.82 mmol) in 1,4-dioxane (60 mL) was added HCl (4 N solution in 1,4-dioxane) (24.54 mL, 98 mmol). The resulting slurry was stirred at room temperature. After 17 h, the reaction mixture was concentrated under reduced pressure and dried to constant weight under high vacuum to afford the title compound (2.63 g, 99%) as a pale yellow solid. MS (ES)⁺ m/e 232.9, 234.8 [M+H]⁺.

### c) 1,1-dimethylethyl(3S)-3-{[({2-[(4-bromo-3-fluorophenyl)carbonyl]hydrazino}carbonyl)amino]methyl}-1-pyrrolidinecarboxylate

To a solution of 1,1-dimethylethyl(3S)-3-(aminomethyl)-1-pyrrolidinecarboxylate (1.000 g, 4.99 mmol, 87% ee) in CH₂Cl₂ (15 mL) was added CDI (0.810 g, 4.99 mmol). The reaction was stirred at ambient temperature under N₂. After 2.5 h, the reaction was concentrated under reduced pressure and the residue was taken up in THF (20 mL), and 4-bromo-3-fluorobenzohydrazide hydrochloride (1.346 g, 4.99 mmol) and DIPEA (1.308 mL, 7.49 mmol) were added. The clear solution was heated to 60° C. After 20 h the reaction mixture was concentrated under reduced pressure and the residue was purified by silica gel chromatography (Analogix, SF25—120 g column, 110 mL/min, 100% EtOAc to 5% MeOH in EtOAc). The fractions containing clean product were concentrated under reduced pressure and dried to constant weight to afford the title compound (1.85 g, 81%) as an off-white foam. MS (ES)⁺ m/e 459.2, 461.3 [M+H]⁺.

### d) 5-(4-bromo-3-fluorophenyl)-4-{[(3R)-1-(cyclopropylcarbonyl)-3-pyrrolidinyl]methyl}-2,4-dihydro-3H-1,2,4-triazol-3-one

To a mixture of 1,1-dimethylethyl(3S)-3-{[({2-[(4-bromo-3-fluorophenyl)carbonyl]hydrazino}carbonyl)amino]methyl}-1-pyrrolidinecarboxylate (1.850 g, 4.03 mmol) in water (80 mL) was added K₂CO₃ (2.78 g, 20.14 mmol) and the mixture was heated to reflux (bath=130° C.) eventually giving a clear solution. After 40 h at reflux, LCMS indicated complete conversion. The reaction was cooled to room temperature, the pH was adjusted to ˜7 with 6 N HCl and the mixture was concentrated in vacuo and dried to constant weight under high vacuum. The residue was diluted with CH₂Cl₂ (40 mL), DIPEA (1.407 mL, 8.06 mmol) was added and the reaction was cooled to 0° C. A solution of cyclopropanecarbonyl chloride (0.375 mL, 4.03 mmol) was added. The resulting suspension was allowed to warm to room temperature under N₂. After 1 h, the reaction was diluted with water and the aqueous layer was back-extracted twice with CH₂Cl₂. The combined extracts were dried, filtered, and concentrated under reduced pressure. The residue was purified by silica gel chromatography (SF25—120 g column, Analogix, 7% MeOH in EtOAc, 110 mL/min flow rate). The appropriate fractions were concentrated and dried to constant weight to afford the title compound (0.855 g, 51.9%) as a tacky white solid. MS (ES)⁺ m/e 409.2, 411.2 [M+H]⁺. This material was purified by prep chiral SFC to afford the title compound in 99.4% ee as a white glassy solid.

### e) 5-[4-(1-benzofuran-5-yl)-3-fluorophenyl]-4-{[(3R)-1-(cyclopropylcarbonyl)-3-pyrrolidinyl]methyl}-2,4-dihydro-3H-1,2,4-triazol-3-one

A sealable reaction tube was charged with 5-(4-bromo-3-fluorophenyl)-4-{[(3R)-1-(cyclopropylcarbonyl)-3-pyrrolidinyl]methyl}-2,4-dihydro-3H-1,2,4-triazol-3-one (150 mg, 0.367 mmol, >99.4% ee), 5-(4,4,5,5-tetramethyl-1,3,2-dioxaborolan-2-yl)-1-benzofuran (94 mg, 0.385 mmol), Pd(Ph₃P)₄ (21.18 mg, 0.018 mmol), Cs₂CO₃ (299 mg, 0.916 mmol), 1,4-dioxane (3 mL), and water (1 mL). The reaction vessel was purged with N₂, sealed, and heat to 100° C. After 16 h at 100° C., LCMS indicated 46% desired product with 12% starting bromide. The reaction mixture was subject to microwave irradiation at 130° C. for 30 min. LCMS shows complete conversion. The reaction was diluted with water and the pH was adjusted to ˜7 with 1 N HCl. The mixture was diluted with brine and extracted twice with EtOAc. The combined extracts were dried over Na₂SO₄, filtered, and concentrated under reduced pressure. The residue was purified by silica gel chromatography (Analogix, SF25—40 g column, 7% MeOH in EtOAc, flow rate=40 mL/min). The appropriate fractions were concentrated and dried to constant weight to afford the title compound (104 mg, 63.6%) as a white solid. MS (ES)⁺ m/e 447.2 [M+H]⁺.

### Example 2

### 5-[4-(1-benzofuran-5-yl)-3-fluorophenyl]-4-{[(3R)-1-(cyclopropylcarbonyl)-3-pyrrolidinyl]methyl}-2-methyl-2,4-dihydro-3H-1,2,4-triazol-3-one

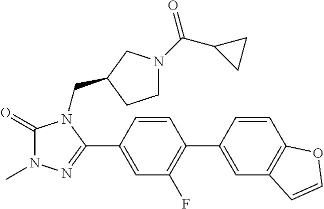 [SMILES: Cn1nc(-c2ccc(-c3ccc4occc4c3)c(F)c2)n(C[C@@H]2CCN(C(=O)C3CC3)C2)c1=O]

A microwave vial was charged with 5-[4-(1-benzofuran-5-yl)-3-fluorophenyl]-4-{[(3R)-1-(cyclopropylcarbonyl)-3-pyrrolidinyl]methyl}-2,4-dihydro-3H-1,2,4-triazol-3-one (63.0 mg, 0.141 mmol), K₂CO₃ (39.0 mg, 0.282 mmol), CH₃CN (2 mL) and iodomethane (0.026 mL, 0.423 mmol). The mixture was purged with N₂, sealed, and heated to 60° C. After 40 h, LCMS indicated ˜60% clean conversion. Another protion of iodomethane (9 μL, 1 equiv.) was added and the reaction was heated to 70° C. After an additional 22 h, LCMS shows 82% clean conversion. The reaction mixture was filtered through a syringe filter and concentrated under reduced pressure. The residue was purified by reverse phase HPLC (10-95% CH₃CN/H₂O+0.1% TFA). The appropriate fractions were adjusted to pH ˜7 with saturated aq. NaHCO₃ and extracted twice with CH₂Cl₂. The organic extracts were dried over Na₂SO₄, filtered, concentrated under reduced pressure, and dried to constant weight in a 50° C. vacuum oven to afford the title compound (37 mg, 56.9%) as an off-white solid. MS (ES)⁺ m/e 461.3 [M+H]⁺. ¹H NMR (400 MHz, DMSO-d₆) δ ppm 8.09 (d, J=1.52 Hz, 1H), 7.91 (br. s., 1H), 7.50-7.81 (m, 5H), 7.06 (s, 1H), 3.81-3.97 (m, 2H), 3.47-3.69 (m, 2H), 3.45 (s, 3H), 2.89-3.32 (m, 2H), 2.39-2.54 (m, 1H), 1.72-1.98 (m, 1H), 1.38-1.70 (m, 2H), 0.57-0.73 (m, 4H).

### Example 3

### 4-{[(3R)-1-(cyclopropylcarbonyl)-3-pyrrolidinyl]methyl}-5-[3-fluoro-4-(1H-indol-5-yl)phenyl]-2,4-dihydro-3H-1,2,4-triazol-3-one

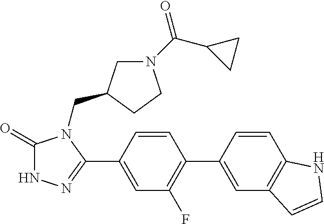 [SMILES: O=C(C1CC1)N1CC[C@@H](Cn2c(-c3ccc(-c4ccc5[nH]ccc5c4)c(F)c3)n[nH]c2=O)C1]

A microwave vial was charged with 5-(4-bromo-3-fluorophenyl)-4-{[(3R)-1-(cyclopropylcarbonyl)-3-pyrrolidinyl]methyl}-2,4-dihydro-3H-1,2,4-triazol-3-one (130 mg, 0.318 mmol, >98% ee), 5-(4,4,5,5-tetramethyl-1,3,2-dioxaborolan-2-yl)-1H-indole (77 mg, 0.318 mmol), Pd(Ph₃P)₄ (18.35 mg, 0.016 mmol), Cs₂CO₃ (259 mg, 0.794 mmol), 1,4-dioxane (3 mL), and water (1 mL). The reaction vessel was purged with N₂, sealed, and heated in a microwave reactor at 130° C. for three 30 min cycles. The reaction was diluted with water and the pH was adjusted to ˜7 with 1 N HCl. The resulting precipitate was collected by filtration and rinsed with 10% EtOAc in hexanes, dissolved in DMSO, and purified by reverse phase HPLC (10-95% CH₃CN/water+0.1% TFA). The appropriate fractions were neutralized with saturated aq. NaHCO₃ and extracted twice with EtOAc. The organic extracts were dried over Na₂SO₄, filtered, and concentrated from 10% EtOAc in hexanes to afford the title compound (35 mg, 24.7%) as a white solid. MS (ES)⁺ m/e 446.3 [M+H]⁺. ¹H NMR (400 MHz, DMSO-d₆) δ ppm 12.09 (br. s., 1H), 11.27 (br. s., 1H), 7.80 (s, 1H), 7.71 (td, J=8.15, 2.15 Hz, 1H), 7.55-7.67 (m, 2H), 7.52 (d, J=8.59 Hz, 1H), 7.43 (t, J=2.65 Hz, 1H), 7.34 (dd, J=8.34, 2.02 Hz, 1H), 6.52 (bs, 1H) 3.77-3.93 (m, 2H), 2.87-3.67 (m, 4H), 2.29-2.49 (m, 1H), 1.73-1.97 (m, 1H), 1.39-1.73 (m, 2H), 0.65 (dd, 4H).

### Example 4

### 4-{[(3R)-1-(cyclopropylcarbonyl)-3-pyrrolidinyl]methyl}-5-[3-fluoro-4-(1H-indol-6-yl)phenyl]-2,4-dihydro-3H-1,2,4-triazol-3-one

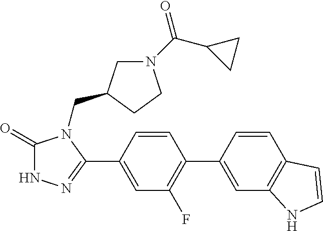 [SMILES: O=C(C1CC1)N1CC[C@@H](Cn2c(-c3ccc(-c4ccc5cc[nH]c5c4)c(F)c3)n[nH]c2=O)C1]

A microwave vial was charged with 5-(4-bromo-3-fluorophenyl)-4-{[(3R)-1-(cyclopropylcarbonyl)-3-pyrrolidinyl]methyl}-2,4-dihydro-3H-1,2,4-triazol-3-one (130 mg, 0.318 mmol, >98% ee), 1H-indol-6-ylboronic acid (51.1 mg, 0.318 mmol), Pd(Ph₃P)₄ (18.35 mg, 0.016 mmol), Cs₂CO₃ (259 mg, 0.794 mmol), 1,4-dioxane (3 mL), and water (1 mL). The reaction vessel was purged with N₂, sealed, and heated in a microwave reactor at 130° C. for 30 min. The reaction was diluted with water and the pH was adjusted to ˜7 with 1 N HCl. The resulting precipitate was collected by filtration and rinsed with 10% EtOAc in hexanes, dissolved in DMSO, and purified by reverse phase HPLC (10-95% CH₃CN/water+0.1% TFA). The appropriate fractions were neutralized with saturated aq. NaHCO₃ and extracted twice with EtOAc. The organic extracts were dried over Na₂SO₄, filtered, concentrated, and dried to constant weight in a 50° C. vacuum oven to afford the title compound (86 mg, 60.8%) as a white solid. MS (ES)⁺ m/e 446.2 [M+H]⁺. ¹H NMR (400 MHz, DMSO-d₆) δ ppm 12.10 (br. s., 1H), 11.30 (br. s., 1H), 7.73 (td, J=8.15, 1.89 Hz, 1H), 7.56-7.70 (m, 4H), 7.46 (t, J=2.78 Hz, 1H), 7.25 (dd, J=8.08, 2.53 Hz, 1H), 6.50 (br. s., 1H), 3.78-3.93 (m, 2H), 2.88-3.68 (m, 4H), 2.29-2.49 (m, 1H), 1.72-1.98 (m, 1H), 1.44-1.71 (m, 2H), 0.58-0.73 (m, 4H).

### Example 5

### 5-[4-(1-benzofuran-5-yl)-2-fluorophenyl]-4-{[(3R)-1-(cyclopropylcarbonyl)-3-pyrrolidinyl]methyl}-2,4-dihydro-3H-1,2,4-triazol-3-one

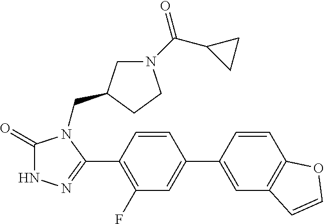 [SMILES: O=C(C1CC1)N1CC[C@@H](Cn2c(-c3ccc(-c4ccc5occc5c4)cc3F)n[nH]c2=O)C1]

### a) 1,1-dimethylethyl 2-[(4-bromo-2-fluorophenyl)carbonyl]hydrazinecarboxylate

To a solution of 4-bromo-2-fluorobenzoic acid (2.50 g, 11.42 mmol) and tert-butyl hydrazinecarboxylate (2.06 g, 11.42 mmol) in DMF (50 mL) were added HOAt (0.155 g, 1.142 mmol) followed by EDC (2.63 g, 13.70 mmol). The reaction was stirred at room temperature under nitrogen. After 2 h, the reaction was diluted with water (150 mL), causing a precipitate to form. After stirring for 10 min, the precipitate was collected by filtration, rinsed with water, and dried to constant weight to afford the title compound (2.94 g, 77%) as a white solid. LC-MS: m/e=333 [M+23]⁺.

### b) 4-bromo-2-fluorobenzohydrazide hydrochloride

To a mixture of 1,1-dimethylethyl 2-[(4-bromo-2-fluorophenyl)carbonyl]hydrazinecarboxylate (2.94 g, 8.82 mmol) in 1,4-dioxane (60 mL) was added HCl (4 N in 1,4-dioxane) (22.06 mL, 88 mmol). The resulting slurry was stirred at room temperature. After 17 h the reaction was diluted with 1,4-dioxane (100 mL) and an additional portion of 4 N HCl (10 mL, 40 mmol) was added. The reaction was stirred for an additional 22 h, then concentrated under reduced pressure and dried to constant weight under high vacuum to afford the title compound (2.37 g, 100%) as a pale yellow solid. MS (ES)⁺ m/e 234.8 [M+H]⁺.

### c) (3R)-1-(cyclopropylcarbonyl)-3-pyrrolidinecarbonitrile

A solution of 1,1-dimethylethyl(3R)-3-cyano-1-pyrrolidinecarboxylate (27 g, 138 mmol) in EtOH (200 mL) was treated with 4 M HCl in 1,4-dioxane (120 mL, 480 mmol) and stirred for 2 h. The mixture was evaporated to an oil, then azeotroped with EtOH and CHCl₃. The residue was taken up in CHCl₃ (300 mL) and DIPEA (71.9 mL, 413 mmol) and cooled over an ice bath. The mixture was treated with cyclopropanecarbonyl chloride (14.98 mL, 165 mmol) in CHCl₃ (100 mL), the ice bath was removed, and the mixture was stirred for 2 h. The mixture was washed with 1 M HCl and brine, dried over Na₂SO₄, and evaporated. Flash chromatography (CH₂Cl₂-5% MeOH in CH₂Cl₂) afforded the title compound (22 g, 97%). LCMS (ES) 165 [M+H]⁺. ¹H NMR (400 MHz, CDCl₃) δ ppm 0.73-0.91 (m, 2H), 0.96-1.10 (m, 2H), 1.47-1.81 (m, 1H), 2.08-2.52 (m, 2H), 3.03-3.33 (m, 1H), 3.48-4.13 (m, 4H).

### d) {[(3S)-1-(cyclopropylcarbonyl)-3-pyrrolidinyl]methyl}amine

A solution of (3R)-1-(cyclopropylcarbonyl)-3-pyrrolidinecarbonitrile (12 g, 73.1 mmol) in EtOH (900 mL) and ammonia solution (45 mL, 73.1 mmol) was divided between 3×500 mL Parr flasks, flushed with N₂, and raney nickel was added. The mixtures were placed on Parr shakers, flushed several times with N₂, and shaken under a hydrogen atmosphere at 60 psi for 3 h. The contents of each flask were flushed with N₂ and filtered through Celite® under a N₂ atmosphere, keeping the catalyst wet, washing through with a little EtOH then immediately dousing with water. The solvent was evaporated to give a clear oil of the desired compound (11.1 g, 90%; 84% pure and ˜88% ee). Purification by preparative chiral HPLC (Chiralpak AD 20μ, heptane:EtOH:isopropylamine—75:25:0.1; UV 220 nm) afforded the title compound (99.4% ee). LCMS (ES) 169 [M+H]⁺; ¹H NMR (400 MHz, CDCl₃) δ ppm 0.69-0.83 (m, 2H), 0.91-1.09 (m, 2H), 1.28 (br. s., 2H), 1.52-1.82 (m, 2H), 1.97-2.20 (m, 1H), 2.20-2.45 (m, 1H), 2.67-2.98 (m, 2H), 3.06-3.38 (m, 1H), 3.38-3.92 (m, 3H).

### e) 2-[(4-bromo-2-fluorophenyl)carbonyl]-N-{[(3S)-1-(cyclopropylcarbonyl)-3-pyrrolidinyl]methyl}hydrazinecarboxamide

To a solution of {[(3S)-1-(cyclopropylcarbonyl)-3-pyrrolidinyl]methyl}amine (800 mg, 4.76 mmol, >95% ee) in CH₂Cl₂ (15 mL) was added CDI (771 mg, 4.76 mmol). The reaction was stirred at amient temperature under N₂. After 16 h, the reaction mixture was concentrated under reduced pressure and the residue was taken up in THF (20 mL) and 4-bromo-2-fluorobenzohydrazide hydrochloride (1282 mg, 4.76 mmol) and DIPEA (1.246 mL, 7.13 mmol) were added. The clear yellow solution was heated to 60° C., and gradually turned into a white slurry. After 7 h, the precipitate was collected by filtration and dried to constant weight to afford the title compound (1750 mg, 86%) as a white solid. MS (ES)⁺ m/e 427.0 [M+H]⁺.

### f) 5-(4-bromo-2-fluorophenyl)-4-{[(3R)-1-(cyclopropylcarbonyl)-3-pyrrolidinyl]methyl}-2,4-dihydro-3H-1,2,4-triazol-3-one

To a mixture of 2-[(4-bromo-2-fluorophenyl)carbonyl]-N-{[(3S)-1-(cyclopropylcarbonyl)-3-pyrrolidinyl]methyl}hydrazinecarboxamide (1.750 g, 4.10 mmol, >95% ee) in water (80 mL) was added K₂CO₃ (2.83 g, 20.48 mmol) and the mixture was heated to reflux eventually giving a clear solution. After 60 h (bath 130° C.), LCMS indicated complete conversion. The reaction was cooled to rt, the pH was adjusted to ˜6.5 with 1 N HCl and the mixture was extracted twice with EtOAc. The combined extracts were dried over Na₂SO₄, filtered, and concentrated. The residue was purified by silica gel chromatography (Analogix, SF40-115 g column, 7% MeOH in EtOAc, flow rate=110 mL/min). The appropriate fractions were concentrated under reduced pressure and dried to constant weight to afford the title compound (0.6677 g, 39.8%) as a white solid. MS (ES)⁺ m/e 409.2, 411.2 [M+H]⁺.

### g) 5-[4-(1-benzofuran-5-yl)-2-fluorophenyl]-4-{[(3R)-1-(cyclopropylcarbonyl)-3-pyrrolidinyl]methyl}-2,4-dihydro-3H-1,2,4-triazol-3-one

A sealable reaction tube was charged with 5-(4-bromo-2-fluorophenyl)-4-{[(3R)-1-(cyclopropylcarbonyl)-3-pyrrolidinyl]methyl}-2,4-dihydro-3H-1,2,4-triazol-3-one (150 mg, 0.367 mmol, >98% ee), 5-(4,4,5,5-tetramethyl-1,3,2-dioxaborolan-2-yl)-1-benzofuran (94 mg, 0.385 mmol), Pd(Ph₃P)₄ (21.18 mg, 0.018 mmol), Cs₂CO₃ (299 mg, 0.916 mmol), 1,4-dioxane (3 mL), and water (1 mL). The reaction vessel was purged with N₂, sealed, and heated to 100° C. After 16 h at 100° C., LCMS indicated 41% desired product with 13% starting bromide. The reaction mixture was subject to microwave irradiation at 130° C. for 15 min. LCMS shows 66% product, 4% starting bromide so it was resubjected to the microwave for 15 min at 130° C. LCMS shows complete conversion. The reaction was diluted with water and the pH was adjusted to ˜7 with 1 N HCl. The mixture was diluted with brine and extracted twice with EtOAc. The combined extracts were dried over Na₂SO₄, filtered, and concentrated under reduced pressure. The residue was purified by silica gel chromatography (Analogix, SF25—40 g column, 7% MeOH in EtOAc, flow rate=40 mL/min). The appropriate fractions were concentrated and dried to constant weight in a 50° C. vacuum oven to afford the title compound (130 mg, 79%) as a yellow solid. MS (ES)⁺ m/e 447.2 [M+H]⁺. ¹H NMR (400 MHz, DMSO-d₆) δ ppm 12.16 (s, 1H), 8.10 (s, 1H), 8.09 (d, J=2.27 Hz, 1H), 7.64-7.86 (m, 5H), 7.05 (d, J=2.02 Hz, 1H), 2.87-3.67 (m, 6H), 2.28-2.49 (m, 1H), 1.69-1.96 (m, 1H), 1.33-1.66 (m, 2H).

### Example 6

### 5-[4-(1-benzofuran-5-yl)-2-fluorophenyl]-4-{[(3R)-1-(cyclopropylcarbonyl)-3-pyrrolidinyl]methyl}-2-methyl-2,4-dihydro-3H-1,2,4-triazol-3-one

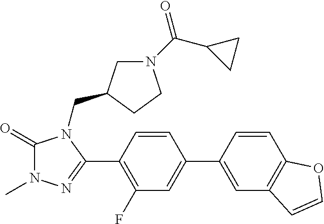 [SMILES: Cn1nc(-c2ccc(-c3ccc4occc4c3)cc2F)n(C[C@@H]2CCN(C(=O)C3CC3)C2)c1=O]

A reaction vial was charged with 5-[4-(1-benzofuran-5-yl)-2-fluorophenyl]-4-{[(3R)-1-(cyclopropylcarbonyl)-3-pyrrolidinyl]methyl}-2,4-dihydro-3H-1,2,4-triazol-3-one (80 mg, 0.179 mmol), K₂CO₃ (49.5 mg, 0.358 mmol), CH₃CN (2 mL) and iodomethane (0.056 mL, 0.896 mmol). The mixture was purged with N₂, sealed, and heated to 60° C. overnight. The reaction was diluted with water and the pH was adjusted to ˜7 with 1 N HCl. The aqueous layer was extracted twice with EtOAc. The organic extracts were dried over Na₂SO₄, filtered, and concentrated under reduced pressure. The residue was purified by silica gel chromatography (3% MeOH in EtOAc) but did not remove a minor impurity, so the material was repurified by reverse phase HPLC (10-95% CH₃CN/H₂O+0.1% TFA). The appropriate fractions were adjusted to pH ˜7 with saturated aq. NaHCO₃ and extracted twice with EtOAc. The organic extracts were dried over Na₂SO₄, filtered, concentrated under reduced pressure, and dried to constant weight in a 50° C. vacuum oven to afford the title compound (34 mg, 41.2%) as a white solid. MS (ES)⁺ m/e 461.4 [M+H]⁺.

### Example 7

### 4-{[(3R)-1-(cyclopropylcarbonyl)-3-pyrrolidinyl]methyl}-5-[2-fluoro-4-(1H-indol-5-yl)phenyl]-2,4-dihydro-3H-1,2,4-triazol-3-one

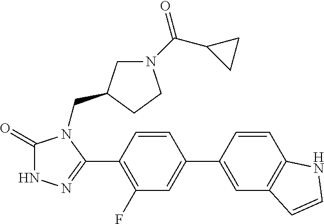 [SMILES: O=C(C1CC1)N1CC[C@@H](Cn2c(-c3ccc(-c4ccc5[nH]ccc5c4)cc3F)n[nH]c2=O)C1]

A microwave vial was charged with 5-(4-bromo-2-fluorophenyl)-4-{[(3R)-1-(cyclopropylcarbonyl)-3-pyrrolidinyl]methyl}-2,4-dihydro-3H-1,2,4-triazol-3-one (100 mg, 0.244 mmol, >98% ee), 5-(4,4,5,5-tetramethyl-1,3,2-dioxaborolan-2-yl)-1H-indole (59.4 mg, 0.244 mmol), Pd(Ph₃P)₄ (14.12 mg, 0.012 mmol), Cs₂CO₃ (199 mg, 0.611 mmol), 1,4-dioxane (3 mL), and water (1 mL). The reaction vessel was purged with N₂, sealed, and heated in a microwave reactor at 130° C. for two 15 min cycles. The reaction was diluted with water and the pH was adjusted to ˜7 with 1 N HCl. The mixture was diluted with brine and extracted twice with EtOAc. The combined extracts were dried over Na₂SO₄, filtered, and concentrated under reduced pressure. The residue was purified by reverse phase HPLC (10-95% CH₃CN/water+0.1% TFA). The appropriate fractions were neutralized with saturated aq. NaHCO₃ and extracted twice with CH₂Cl₂. The organic extracts were dried over Na₂SO₄, filtered, and concentrated from 10% EtOAc in hexanes to afford the title compound (33 mg, 30.3%) as an off-white solid. MS (ES)⁺ m/e 446.2 [M+H]⁺. ¹H NMR (400 MHz, DMSO-d₆) δ ppm 12.13 (br. s., 1H), 11.27 (br. s., 1H), 8.00 (s, 1H), 7.58-7.83 (m, 3H), 7.46-7.57 (m, 2H), 7.42 (t, J=2.78 Hz, 1H), 6.52 (br. s., 1H), 2.75-3.73 (m, 6H), 2.23-2.49 (m, 1H), 1.32-2.02 (m, 3H), 0.53-0.75 (m, 4H).

### Example 8

### 4-{[(3R)-1-(cyclopropylcarbonyl)-3-pyrrolidinyl]methyl}-5-[2-fluoro-4-(1H-indol-6-yl)phenyl]-2,4-dihydro-3H-1,2,4-triazol-3-one

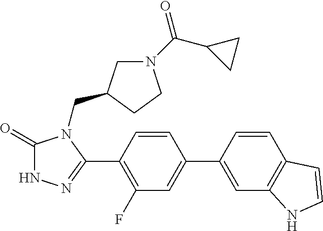 [SMILES: O=C(C1CC1)N1CC[C@@H](Cn2c(-c3ccc(-c4ccc5cc[nH]c5c4)cc3F)n[nH]c2=O)C1]

A microwave vial was charged with 5-(4-bromo-2-fluorophenyl)-4-{[(3R)-1-(cyclopropylcarbonyl)-3-pyrrolidinyl]methyl}-2,4-dihydro-3H-1,2,4-triazol-3-one (130 mg, 0.318 mmol, >98% ee), 1H-indol-6-ylboronic acid (53.7 mg, 0.334 mmol), Pd(Ph₃P)₄ (18.35 mg, 0.016 mmol), Cs₂CO₃ (259 mg, 0.794 mmol), 1,4-dioxane (3 mL), and water (1 mL). The reaction vessel was purged with N₂, sealed, and heated in a microwave reactor at 130° C. for 30 min. The reaction was diluted with water and the pH was adjusted to ˜7 with 1 N HCl. The mixture was diluted with brine and extracted twice with EtOAc. The combined extracts were dried over Na₂SO₄, filtered, and concentrated under reduced pressure. The residue was purified by reverse phase HPLC (10-95% CH₃CN/water+0.1% TFA). The appropriate fractions were concentrated to remove a majority of the CH₃CN and the remaining aqueous mixture was neutralized with saturated aq. NaHCO₃ and extracted twice with EtOAc. The organic extracts were dried over Na₂SO₄, filtered, and concentrated under reduced pressure. The residue was triturated with 10% EtOAc in hexanes, and the solid was collected by filtration and dried to constant weight in a 50° C. vacuum oven to afford the title compound (84 mg, 59.4%) as a white solid. MS (ES)⁺ m/e 446.2 [M+H]⁺. ¹H NMR (400 MHz, DMSO-d₆) δ ppm 12.14 (br. s., 1H), 11.31 (br. s., 1H), 7.60-7.84 (m, 5H), 7.34-7.51 (m, 2H), 6.49 (br. s., 1H), 2.85-3.71 (m, 6H), 2.30-2.48 (m, 1H), 1.70-1.95 (m, 1H), 1.35-1.67 (m, 2H), 0.56-0.71 (m, 4H).

### Example 9

### N′-[4′-(4-{[(3R)-1-(cyclopropylcarbonyl)-3-pyrrolidinyl]methyl}-5-oxo-4,5-dihydro-1H-1,2,4-triazol-3-yl)-3′-fluoro-3-biphenylyl]-N,N-dimethylsulfamide

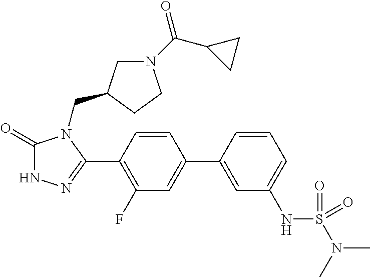 [SMILES: CN(C)S(=O)(=O)Nc1cccc(-c2ccc(-c3n[nH]c(=O)n3C[C@@H]3CCN(C(=O)C4CC4)C3)c(F)c2)c1]

A microwave vial was charged with 5-(4-bromo-2-fluorophenyl)-4-{[(3R)-1-(cyclopropylcarbonyl)-3-pyrrolidinyl]methyl}-2,4-dihydro-3H-1,2,4-triazol-3-one (100 mg, 0.244 mmol, >98% ee), (3-{[(dimethylamino)sulfonyl]amino}phenyl)boronic acid (62.6 mg, 0.257 mmol), Pd(Ph₃P)₄ (14.12 mg, 0.012 mmol), Cs₂CO₃ (199 mg, 0.611 mmol), 1,4-dioxane (3 mL), and water (1 mL). The reaction vessel was purged with N₂, sealed, and heated in a microwave reactor at 130° C. for 30 min. The reaction was concentrated under reduced pressure, taken up in DMSO (3 mL), filtered through an Acrodisc® syringe filter and purified by reverse phase HPLC (10-95% CH₃CN/water+0.1% TFA). The appropriate fractions were concentrated to remove a majority of the CH₃CN and the remaining aqueous mixture was neutralized with saturated aq. NaHCO₃ and extracted twice with EtOAc. The organic extracts were dried over Na₂SO₄, filtered, and concentrated under reduced pressure. The residue was concentrated from 10% EtOAc in hexanes (helped to solidify), and the solid was dried to constant weight in a 50° C. vacuum oven to afford the title compound (66 mg, 51.1%) as a white solid. MS (ES)⁺ m/e 529.1 [M+H]⁺. ¹H NMR (400 MHz, DMSO-d₆) δ ppm 12.17 (s, 1H), 10.08 (br. s., 1H), 7.57-7.82 (m, 3H), 7.54 (s, 1H), 7.41-7.51 (m, 2H), 7.28 (dt, J=7.01, 2.05 Hz, 1H), 2.81-3.76 (m, 6H), 2.74 (s, 6H), 2.29-2.48 (m, 1H), 1.70-1.96 (m, 1H), 1.35-1.66 (m, 2H), 0.56-0.72 (m, 4H).

### Example 10

### N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3′-fluoro-3-biphenylyl]-N,N-dimethylsulfamide

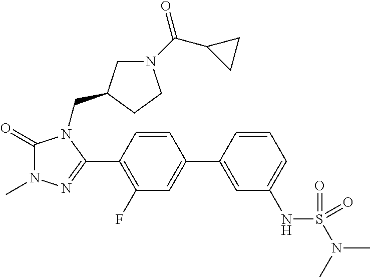 [SMILES: CN(C)S(=O)(=O)Nc1cccc(-c2ccc(-c3nn(C)c(=O)n3C[C@@H]3CCN(C(=O)C4CC4)C3)c(F)c2)c1]

### a) 5-(4-bromo-2-fluorophenyl)-4-{[(3R)-1-(cyclopropylcarbonyl)-3-pyrrolidinyl]methyl}-2-methyl-2,4-dihydro-3H-1,2,4-triazol-3-one

A reaction vial was charged with 5-(4-bromo-2-fluorophenyl)-4-{[(3R)-1-(cyclopropylcarbonyl)-3-pyrrolidinyl]methyl}-2,4-dihydro-3H-1,2,4-triazol-3-one (176 mg, 0.430 mmol), K₂CO₃ (119 mg, 0.860 mmol), CH₃CN (4 mL) and iodomethane (0.081 mL, 1.290 mmol). The mixture was purged with N₂, sealed, and heated to 60° C. After 2.5 h, LCMS shows ˜60% conversion, so the reaction temperature was increased to 80° C. After 20 h, LCMS indicated complete conversion. The reaction was diluted with water and extracted thrice with EtOAc. The combined extracts were dried over Na₂SO₄, filtered, and concentrated under reduced pressure. The residue was purified by silica gel chromatography (Analogix, 5% MeOH in EtOAc) to afford the title compound (141 mg, 77%) as a tacky white solid. MS (ES)⁺ m/e 422.8, 424.8 [M+H]⁺.

### b) N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3′-fluoro-3-biphenylyl]-N,N-dimethylsulfamide

A microwave vial was charged with 5-(4-bromo-2-fluorophenyl)-4-{[(3R)-1-(cyclopropylcarbonyl)-3-pyrrolidinyl]methyl}-2-methyl-2,4-dihydro-3H-1,2,4-triazol-3-one (75 mg, 0.177 mmol, >98% ee), (3-{[(dimethylamino)sulfonyl]amino}phenyl)boronic acid (45.4 mg, 0.186 mmol), Pd(Ph₃P)₄ (10.24 mg, 8.86 μmol), K₂CO₃ (61.2 mg, 0.443 mmol), 1,4-dioxane (3 mL), and water (1 mL). The reaction vessel was purged with N₂, sealed, and heated in a microwave reactor at 130° C. for 30 min. The reaction was concentrated under reduced pressure, taken up in DMSO (2 mL), filtered through an Acrodisc® syringe filter and purified by reverse phase HPLC (10-95% CH₃CN/water+0.1% TFA). The appropriate fractions were concentrated to remove a majority of the CH₃CN and the remaining aqueous mixture was neutralized with saturated aq. NaHCO₃ and extracted twice with CH₂Cl₂. The organic extracts were dried over Na₂SO₄, filtered, and concentrated under reduced pressure. The residue was redissolved in CH₂Cl₂, concentrated under reduced pressure, and dried to constant weight in a 50° C. vacuum oven to afford the title compound (64 mg, 66.6%) as a white solid. MS (ES)⁺ m/e 543.2 [M+H]⁺.

### Example 11

### 4-{[(3R)-1-(cyclopropylcarbonyl)-3-pyrrolidinyl]methyl}-2-methyl-5-[2′,3,5′-trifluoro-4′-(methyloxy)-4-biphenylyl]-2,4-dihydro-3H-1,2,4-triazol-3-one

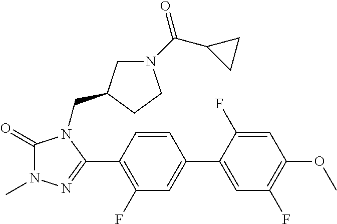 [SMILES: COc1cc(F)c(-c2ccc(-c3nn(C)c(=O)n3C[C@@H]3CCN(C(=O)C4CC4)C3)c(F)c2)cc1F]

A microwave vial was charged with 5-(4-bromo-2-fluorophenyl)-4-{[(3R)-1-(cyclopropylcarbonyl)-3-pyrrolidinyl]methyl}-2-methyl-2,4-dihydro-3H-1,2,4-triazol-3-one (75 mg, 0.177 mmol, >98% ee), [2,5-difluoro-4-(methyloxy)phenyl]boronic acid (33.3 mg, 0.177 mmol), Pd(Ph₃P)₄ (10.24 mg, 8.86 μmol), K₂CO₃ (61.2 mg, 0.443 mmol), 1,4-dioxane (3 mL), and water (1 mL). The reaction vessel was purged with N₂, sealed, and heated in a microwave reactor at 130° C. for 30 min. The reaction was concentrated under reduced pressure, taken up in DMSO (2 mL), filtered through an Acrodisc® syringe filter, and purified by reverse phase HPLC (10-95% CH₃CN/water+0.1% TFA). The appropriate fractions were concentrated to remove a majority of the CH₃CN and the remaining aqueous mixture was neutralized with saturated aq. NaHCO₃ and extracted twice with CH₂Cl₂. The organic extracts were dried over Na₂SO₄, filtered, concentrated under reduced pressure, and dried to constant weight in a 50° C. vacuum oven to afford the title compound (44 mg, 51.0%) as a white solid. MS (ES)⁺ m/e 487.2 [M+H]⁺.

### Example 12

### 5-[4-(1-benzofuran-5-yl)-2-methylphenyl]-4-{[(3R)-1-(cyclopropylcarbonyl)-3-pyrrolidinyl]methyl}-2,4-dihydro-3H-1,2,4-triazol-3-one

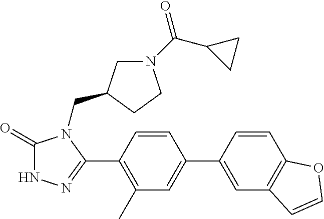 [SMILES: Cc1cc(-c2ccc3occc3c2)ccc1-c1n[nH]c(=O)n1C[C@@H]1CCN(C(=O)C2CC2)C1]

### a) tert-butyl 2-(4-bromo-2-methylbenzoyl)hydrazinecarboxylate

A mixture of 4-bromo-2-methylbenzoic acid (8.6 g, 40 mmol), tert-butyl hydrazinecarboxylate (5.8 g, 44 mmol), EDC (15.3 g, 80 mmol), HOAt (10.9 g, 80 mmol), and DIPEA (10.3 g, 80 mmol) in DMF (200 mL) was stirred overnight at room temperature. The mixture was poured into water and the resulting precipitate was collected by filtration and dried to afford the title compound (9.6 g, 72%) as a white solid. ¹H NMR (400 MHZ, CDCl₃): δ ppm 8.16 (s, 1H), 7.40-7.35 (m, 3H), 6.86 (s, 1H), 2.46 (s, 3H), 1.50 (s, 9H); LC-MS: m/e=352 [M+23]⁺.

### b) 4-bromo-2-methylbenzohydrazide

A solution of tert-butyl 2-(4-bromo-2-methylbenzoyl)hydrazinecarboxylate (8 g, 24.3 mmol) in 4 N aq. HCl (100 mL) and 1,4-dioxane (100 mL) was stirred at room temperature for 4 h. The reaction mixture was concentrated partially and the precipitate (white solid) was collected by filtration. The solid was added to saturated aq. NaHCO₃ (200 mL), stirred, and collected by filtration to afford the title compound (4 g, 72%) as a yellow solid. ¹H NMR (400 MHZ, CDCl₃): δ ppm 7.40-7.22 (m, 3H), 2.43 (s, 3H); LC-MS: m/e=231 [M+1]⁺.

### c) (S)-2-(4-bromo-2-methylbenzoyl)-N-((1-(cyclopropanecarbonyl)pyrrolidin-3-yl)methyl)hydrazinecarboxamide

A solution of 4-bromo-2-methylbenzohydrazide (4 g, 15 mmol) and (S)—N-((1-(cyclopropanecarbonyl)pyrrolidin-3-yl)methyl)-1H-imidazole-1-carboxamide (4.35 g, 16.6 mmol) in THF (200 mL) was stirred overnight at 65° C. The reaction mixture was cooled and the resulting precipitate was collected by filtration to afford the title compound (3.4 g, 54%) as a white solid. ¹H NMR (400 MHZ, DMSO-d₆): δ ppm 9.85 (s, 1H), 7.93-7.90 (m, 1H), 7.52-7.39 (m, 3H), 6.67-6.59 (m, 1H), 3.74-2.98 (m, 5H), 2.48-2.25 (m, 2H), 2.37 (s, 3H), 2.03-1.51 (m, 3H), 0.72-0.69 (m, 4H); LC-MS: m/e=425 [M+1]⁺.

### d) 5-[4-(1-benzofuran-5-yl)-2-methylphenyl]-4-{[(3R)-1-(cyclopropylcarbonyl)-3-pyrrolidinyl]methyl}-2,4-dihydro-3H-1,2,4-triazol-3-one

A mixture of (S)-2-(4-bromo-2-methylbenzoyl)-N-((1-(cyclopropanecarbonyl)pyrrolidin-3-yl)methyl)hydrazinecarboxamide (3.4 g, 8 mmol) in 0.05 M aq. K₂CO₃ (1 L) was stirred at 120° C. for 1 d, then cooled and extracted with EtOAc (3×200 mL). The organic layer was dried over Na₂SO₄ and concentrated under reduced pressure. The residue was diluted with CH₂Cl₂ and a solid, determined to be starting material (0.72 g), was filtered away from the filtrate. The filtrate was purified by silica gel chromatography (eluted with CH₂Cl₂:MeOH=25:1) to afford the title compound (1.2 g, 46%). ¹H NMR (400 MHZ, CDCl₃): δ ppm 10.59 (s, 1H), 7.53-7.45 (M, 2H), 7.19-7.16 (m, 1H), 3.71-2.97 (m, 6H), 2.62-2.44 (m, 1H), 2.28 (S, 3H), 2.04-1.41 (M, 3H), 1.01-0.91 (m, 2H), 0.75-0.72 (m, 2H); LC-MS: m/e=407 [M+1]⁺.

### e) 5-[4-(1-benzofuran-5-yl)-2-methylphenyl]-4-{[(3R)-1-(cyclopropylcarbonyl)-3-pyrrolidinyl]methyl}-2,4-dihydro-3H-1,2,4-triazol-3-one

A microwave vial was charged with 5-(4-bromo-2-methylphenyl)-4-{[(3R)-1-(cyclopropylcarbonyl)-3-pyrrolidinyl]methyl}-2,4-dihydro-3H-1,2,4-triazol-3-one (150 mg, 0.370 mmol, >98% ee), 5-(4,4,5,5-tetramethyl-1,3,2-dioxaborolan-2-yl)-1-benzofuran (95 mg, 0.389 mmol), Pd(Ph₃P)₄ (21.38 mg, 0.019 mmol), K₂CO₃ (128 mg, 0.925 mmol), 1,4-dioxane (3 mL), and water (1 mL). The reaction vessel was purged with N₂, sealed, and heated in a microwave reactor at 150° C. for 30 min. The reaction mixture was concentrated under reduced pressure and the residue was dissolved in DMSO, filtered through a syringe filter, and purified by reverse phase HPLC (10-95% CH₃CN/water+0.1% TFA). The appropriate fractions were concentrated under reduced pressure to remove a majority of the CH₃CN, leaving an aqueous mixture which was neutralized with saturated aq. NaHCO₃ and extracted twice with CH₂Cl₂. The organic extracts were dried over Na₂SO₄, filtered, concentrated, and dried to constant weight in a 50° C. vacuum oven to afford the desired compound as a white solid. LCMS shows 5% triphenylphosphine oxide, so the material was triturated and sonicated with 50% EtOAc/hexanes and allowed to stand overnight. The tacky solid was collected by filtration, rinsed with 50% EtOAc/hexanes, dissolved in CH₂Cl₂, concentrated under reduced pressure, and dried to constant weight to afford the title compound (72 mg, 44.0%) as a white solid. MS (ES)⁺ m/e 443.2 [M+H]⁺. ¹H NMR (400 MHz, DMSO-d₆) δ ppm 11.97 (s, 1H), 7.95-8.12 (m, 2H), 7.45-7.83 (m, 5H), 7.04 (s, 1H), 3.44-3.65 (m, 3H), 2.80-3.31 (m, 3H), 2.22-2.49 (m, 4H), 1.68-1.96 (m, 1H), 1.29-1.68 (m, 2H), 0.64 (d, 4H).

### Example 13

### N′-[4′-(4-{[(3R)-1-(cyclopropylcarbonyl)-3-pyrrolidinyl]methyl}-5-oxo-4,5-dihydro-1H-1,2,4-triazol-3-yl)-3′-methyl-3-biphenylyl]-N,N-dimethylsulfamide

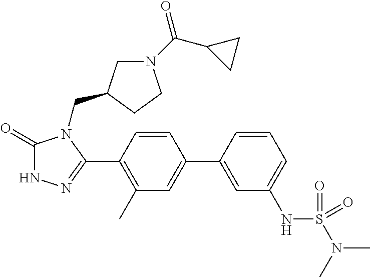 [SMILES: Cc1cc(-c2cccc(NS(=O)(=O)N(C)C)c2)ccc1-c1n[nH]c(=O)n1C[C@@H]1CCN(C(=O)C2CC2)C1]

A microwave vial was charged with 5-(4-bromo-2-methylphenyl)-4-{[(3R)-1-(cyclopropylcarbonyl)-3-pyrrolidinyl]methyl}-2,4-dihydro-3H-1,2,4-triazol-3-one (150 mg, 0.370 mmol, >98% ee), (3-{[(dimethylamino)sulfonyl]amino}phenyl)boronic acid (95 mg, 0.389 mmol), Pd(Ph₃P)₄ (21.38 mg, 0.019 mmol), K₂CO₃ (128 mg, 0.925 mmol), 1,4-dioxane (3 mL), and water (1 mL). The reaction vessel was purged with N₂, sealed, and heated in a microwave reactor at 150° C. for 30 min. The reaction mixture was concentrated under reduced pressure and the residue was dissolved in DMSO, filtered through a syringe filter, and purified by reverse phase HPLC (10-95% CH₃CN/water+0.1% TFA). The appropriate fractions were concentrated under reduced pressure to remove a majority of the CH₃CN, leaving an aqueous mixture which was neutralized with saturated aq. NaHCO₃ and extracted twice with CH₂Cl₂. The organic extracts were dried over Na₂SO₄, filtered, concentrated, and dried to constant weight in a 50° C. vacuum oven to afford the title compound as an off-white solid. MS (ES)⁺ m/e 525.1 [M+H]⁺. ¹H NMR (400 MHz, DMSO-d₆) δ ppm 11.98 (s, 1H), 9.86-10.26 (m, 1H), 7.37-7.66 (m, 6H), 7.21-7.28 (m, 1H), 3.49-2.86 (m, 6H), 2.73 (s, 6H), 2.32 (s, 4H), 1.69-1.94 (m, 1H), 1.31-1.68 (m, 2H), 0.57-0.74 (m, 4H).

### Example 14

### 4-{[(3R)-1-(cyclopropylcarbonyl)-3-pyrrolidinyl]methyl}-5-[4-(1H-indol-6-yl)-2-methylphenyl]-2,4-dihydro-3H-1,2,4-triazol-3-one

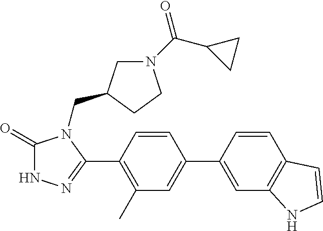 [SMILES: Cc1cc(-c2ccc3cc[nH]c3c2)ccc1-c1n[nH]c(=O)n1C[C@@H]1CCN(C(=O)C2CC2)C1]

A microwave vial was charged with 5-(4-bromo-2-methylphenyl)-4-{[(3R)-1-(cyclopropylcarbonyl)-3-pyrrolidinyl]methyl}-2,4-dihydro-3H-1,2,4-triazol-3-one (150 mg, 0.370 mmol, >98% ee), 1H-indol-6-ylboronic acid (62.6 mg, 0.389 mmol), Pd(Ph₃P)₄ (21.38 mg, 0.019 mmol), K₂CO₃ (128 mg, 0.925 mmol), 1,4-dioxane (3 mL), and water (1 mL). The reaction vessel was purged with N₂, sealed, and heated in a microwave reactor at 130° C. for 30 min. LCMS showed incomplete reaction, so the mixture was resubjected to the microwave for 30 min at 150° C. The reaction mixture was concentrated under reduced pressure and the residue was dissolved in DMSO, filtered through a syringe filter, and purified by reverse phase HPLC (10-95% CH₃CN/water+0.1% TFA). The appropriate fractions were concentrated under reduced pressure to remove a majority of the CH₃CN, leaving an aqueous suspension which was neutralized with saturated aq. NaHCO₃ and extracted twice with CH₂Cl₂. The organic extracts were dried over Na₂SO₄, filtered, concentrated, and dried to constant weight in a 50° C. vacuum oven to afford the title compound (104 mg, 63.6%) as an off-white solid. MS (ES)⁺ m/e 442.2 [M+H]⁺. ¹H NMR (400 MHz, DMSO-d₆) δ ppm 11.95 (s, 1H), 11.23 (br. s., 1H), 7.72 (br. s., 2H), 7.65 (d, J=8.34 Hz, 2H), 7.35-7.56 (m, 3H), 6.47 (br. s., 1H), 3.60-2.88 (m, 6H), 2.24-2.49 (m, 4H), 1.68-1.96 (m, 1H), 1.29-1.68 (m, 2H), 0.54-0.76 (m, 4H).

### Example 15

### 4-{[(3R)-1-(cyclopropylcarbonyl)-3-pyrrolidinyl]methyl}-5-(4′-fluoro-3-methyl-4-biphenylyl)-2,4-dihydro-3H-1,2,4-triazol-3-one

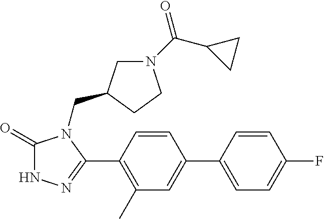 [SMILES: Cc1cc(-c2ccc(F)cc2)ccc1-c1n[nH]c(=O)n1C[C@@H]1CCN(C(=O)C2CC2)C1]

A microwave vial was charged with 5-(4-bromo-2-methylphenyl)-4-{[(3R)-1-(cyclopropylcarbonyl)-3-pyrrolidinyl]methyl}-2,4-dihydro-3H-1,2,4-triazol-3-one (150 mg, 0.370 mmol, >98% ee), (4-fluorophenyl)boronic acid (51.8 mg, 0.370 mmol), PdCl₂(dppf) (13.54 mg, 0.019 mmol), K₂CO₃ (128 mg, 0.925 mmol), 1,4-dioxane (3 mL), and water (1 mL). The reaction vessel was purged with N₂, sealed, and heated in a microwave reactor at 150° C. for 30 min. The reaction mixture was concentrated under reduced pressure and the residue was dissolved in DMSO, filtered through a syringe filter, and purified by reverse phase HPLC (10-95% CH₃CN/water+0.1% TFA). The appropriate fractions were concentrated under reduced pressure to remove a majority of the CH₃CN, leaving an aqueous mixture which was neutralized with saturated aq. NaHCO₃ and extracted twice with CH₂Cl₂. The organic extracts were dried over Na₂SO₄, filtered, concentrated, and dried to constant weight in a 50° C. vacuum oven to afford the title compound as a white solid. MS (ES)⁺ m/e 421.1 [M+H]⁺. ¹H NMR (400 MHz, DMSO-d₆) δ ppm 11.97 (s, 1H), 7.75-7.87 (m, 2H), 7.69-7.74 (m, 1H), 7.59-7.66 (m, 1H), 7.52 (dd, J=18.32, 7.96 Hz, 1H), 7.33 (t, J=8.59 Hz, 2H), 3.59-2.85 (m, 6H), 2.24-2.48 (m, 4H), 1.69-1.93 (m, 1H), 1.29-1.66 (m, 2H), 0.55-0.72 (m, 4H).

### Example 16

### 4-{[(3R)-1-(cyclopropylcarbonyl)-3-pyrrolidinyl]methyl}-5-[4-(1H-indol-5-yl)-2-methylphenyl]-2,4-dihydro-3H-1,2,4-triazol-3-one

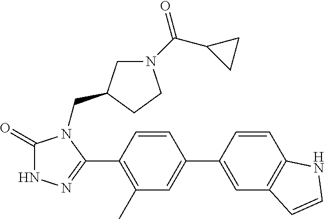 [SMILES: Cc1cc(-c2ccc3[nH]ccc3c2)ccc1-c1n[nH]c(=O)n1C[C@@H]1CCN(C(=O)C2CC2)C1]

A microwave vial was charged with 5-(4-bromo-2-methylphenyl)-4-{[(3R)-1-(cyclopropylcarbonyl)-3-pyrrolidinyl]methyl}-2,4-dihydro-3H-1,2,4-triazol-3-one (150 mg, 0.370 mmol, >98% ee), 5-(4,4,5,5-tetramethyl-1,3,2-dioxaborolan-2-yl)-1H-indole (90 mg, 0.370 mmol), PdCl₂(dppf) (13.54 mg, 0.019 mmol), K₂CO₃ (128 mg, 0.925 mmol), 1,4-dioxane (3 mL), and water (1 mL). The reaction vessel was purged with N₂, sealed, and heated in a microwave reactor at 150° C. for 30 min. The reaction mixture was diluted with water, adjusted to pH ˜6.5, and extracted twice with CH₂Cl₂. The combined extracts were dried over Na₂SO₄, filtered, and concentrated under reduced pressure. The residue was purifed by silica gel chromatography (Analogix, 40 g column, 7% MeOH in EtOAc). The appropriate fractions were concentrated and the resulting precipitate was triturated with hot 35% EtOAc/hexanes, collected by filtration, and dried to constant weight to afford the title compound (86 mg, 52.6%) as a white solid. MS (ES)⁺ m/e 442.2 [M+H]⁺. ¹H NMR (400 MHz, DMSO-d₆) δ ppm 11.95 (s, 1H), 11.21 (br. s., 1H), 7.92 (s, 1H), 7.72 (d, J=3.28 Hz, 1H), 7.59-7.68 (m, 1H), 7.35-7.56 (m, 4H), 6.51 (br. s., 1H), 3.59-2.87 (m, 6H), 2.25-2.49 (m, 4H), 1.68-1.93 (m, 1H), 1.33-1.68 (m, 2H), 0.55-0.72 (m, 4H).

### Example 17

### 5-[2-chloro-4-(1H-indol-6-yl)phenyl]-4-{[(3R)-1-(cyclopropylcarbonyl)-3-pyrrolidinyl]methyl}-2,4-dihydro-3H-1,2,4-triazol-3-one

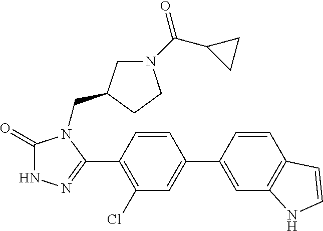 [SMILES: O=C(C1CC1)N1CC[C@@H](Cn2c(-c3ccc(-c4ccc5cc[nH]c5c4)cc3Cl)n[nH]c2=O)C1]

### a) tert-butyl 2-(4-bromo-2-chlorobenzoyl)hydrazinecarboxylate

A mixture of 4-bromo-2-chlorobenzoic acid (4.7 g, 20 mmol), EDC (7.7 g, 40 mmol), HOAt (5.4 g, 40 mmol), and DIPEA (5.2 g, 40 mmol) in DMF (100 mL) was stirred at room temperature for 10 min. tert-Butyl hydrazinecarboxylate (2.9 g, 22 mmol) was added and the resulting mixture was further stirred overnight at room temperature. The mixture was poured into water, filtered, and dried to afford the title compound (6 g, 86%) as a white solid. ¹H NMR (400 MHZ, CDCl₃): δ ppm 7.95 (s, 1H), 7.64-7.61 (m, 2H), 7.51-7.48 (m, 1H), 6.72 (s, 1H), 1.51 (s, 9H); LC-MS: m/e=373 [M+23]⁺.

### b) 4-bromo-2-chlorobenzohydrazide hydrochloride

A solution of tert-butyl 2-(4-bromo-2-chlorobenzoyl)hydrazinecarboxylate (6 g, 17 mmol) in saturated HCl (g) in EtOAc (60 mL), was stirred overnight at room temperature. The reaction mixture was filtered and washed with CH₂Cl₂ to afford the title compound (4.3 g, 87%). ¹H NMR (400 MHZ, D₂O): δ ppm 7.57 (d, J=1.2 Hz, 1H), 7.39 (dd, J=1.2, 8.4 Hz, 1H), 7.20 (d, J=8.4 Hz, 1H); LC-MS: m/e=251 [M+1]⁺.

### c) (S)—N-((1-(cyclopropanecarbonyl)pyrrolidin-3-yl)methyl)-1H-imidazole-1-carboxamide

To a solution of (S)-(3-(aminomethyl)pyrrolidin-1-yl)(cyclopropyl)methanone (5 g, 29.8 mmol) in CH₂Cl₂ (1000 mL), was added CDI (5.86 g, 35.7 mmol) in one batch, then the mixture was stirred overnight at room temperature. The mixture was concentrated and the residue was purified by silica gel chromatography (eluted with CH₂Cl₂:MeOH=30:1), to afford the title compound (5 g) as an oil with some CDI as impurity. LC-MS: m/e=263 [M+1]⁺.

### d) (S)-2-(4-bromo-2-chlorobenzoyl)-N-((1-(cyclopropanecarbonyl)pyrrolidin-3-yl)methyl)hydrazinecarboxamide

To a solution of 4-bromo-2-chlorobenzohydrazide hydrochloride (2.5 g, 9.4 mmol) and DIPEA (1.2 g, 9.4 mmol) in THF (100 mL), was added (S)—N-((1-(cyclopropanecarbonyl)pyrrolidin-3-yl)methyl)-1H-imidazole-1-carboxamide (2.46 g, 9.4 mmol). The mixture was stirred overnight at 65° C., cooled, and the precipitate was collected by filtration to afford the title compound (1.2 g, 32%) as a white solid. ¹H NMR (400 MHZ, DMSO-d₆): δ ppm 9.85 (s, 1H), 7.93-7.89 (m, 1H), 7.51-7.37 (m, 3H), 6.65-6.59 (m, 1H), 3.72-2.96 (m, 5H), 2.51-2.36 (m, 2H), 1.98-1.52 (m, 3H), 0.71-0.68 (m, 4H); LC-MS: m/e=445 [M+1]⁺.

### e) 5-(4-bromo-2-chlorophenyl)-4-{[(3R)-1-(cyclopropylcarbonyl)-3-pyrrolidinyl]methyl}-2,4-dihydro-3H-1,2,4-triazol-3-one

A mixture of (S)-2-(4-bromo-2-chlorobenzoyl)-N-((1-(cyclopropanecarbonyl)pyrrolidin-3-yl)methyl)hydrazinecarboxamide (1.5 g, 3.37 mmol) in 0.05 M aq. K₂CO₃ (500 mL) was stirred at 120° C. for 3 d. After cooling, the mixture was extracted with EtOAc (3×200 mL) and the organic layer was washed with brine, dried over Na₂SO₄, and concentrated. The residue was purified by silica gel chromatography (eluted with CH₂Cl₂:MeOH=25:1) to afford the title compound (0.5 g, 35%). ¹H NMR (400 MHZ, CDCl₃): δ ppm 10.02 (s, 1H), 7.72 (s, 1H), 7.60-7.57 (m, 1H), 7.37-7.32 (m, 1H), 3.74-2.95 (m, 6H), 2.65-2.48 (m, 1H), 2.06-1.42 (m, 3H), 0.98-0.94 (m, 2H), 0.76-0.72 (m, 2H); LC-MS: m/e=427 [M+1]⁺. This material was further purified by prep chiral SFC to afford the title compound in >95% ee.

### f) 5-[2-chloro-4-(1H-indol-6-yl)phenyl]-4-{[(3R)-1-(cyclopropylcarbonyl)-3-pyrrolidinyl]methyl}-2,4-dihydro-3H-1,2,4-triazol-3-one

A microwave vial was charged with 5-(4-bromo-2-chlorophenyl)-4-{[(3R)-1-(cyclopropylcarbonyl)-3-pyrrolidinyl]methyl}-2,4-dihydro-3H-1,2,4-triazol-3-one (150 mg, 0.352 mmol, >98% ee), 1H-indol-6-ylboronic acid (59.6 mg, 0.370 mmol), PdCl₂(dppf) (12.89 mg, 0.018 mmol), K₂CO₃ (122 mg, 0.881 mmol), 1,4-dioxane (3 mL), and water (1 mL). The reaction vessel was purged with N₂, sealed, and heated in a microwave reactor at 150° C. for 30 min. LCMS showed incomplete reaction, so the mixture was resubjected to the microwave for 30 min at 150° C. The reaction mixture was diluted with water, adjusted to pH ˜6.5, and extracted twice with CH₂Cl₂. The combined extracts were dried over Na₂SO₄, filtered, and concentrated under reduced pressure. The residue was purifed by silica gel chromatography (Analogix, 40 g column, 7% MeOH in EtOAc). The appropriate fractions were concentrated and the resulting precipitate was triturated with hot 35% EtOAc/hexanes, collected by filtration, and dried to constant weight to afford the title compound (102 mg, 62.7%) as a white solid. MS (ES)⁺ m/e 462.2 [M+H]⁺. ¹H NMR (400 MHz, DMSO-d₆) δ ppm 12.09 (s, 1H), 11.30 (br. s., 1H), 7.96 (dd, J=4.29, 1.77 Hz, 1H), 7.83 (ddd, J=7.83, 5.81, 1.77 Hz, 1H), 7.78 (s, 1H), 7.62-7.75 (m, 2H), 7.38-7.48 (m, 2H), 6.49 (br. s., 1H), 3.63-2.89 (m, 6H), 2.27-2.48 (m, 1H), 1.72-1.94 (m, 1H), 1.36-1.68 (m, 2H), 0.56-0.72 (m, 4H).

### Example 18

### 5-[2-chloro-4-(1H-indol-5-yl)phenyl]-4-{[(3R)-1-(cyclopropylcarbonyl)-3-pyrrolidinyl]methyl}-2,4-dihydro-3H-1,2,4-triazol-3-one

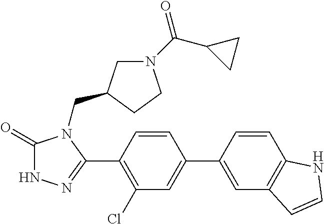 [SMILES: O=C(C1CC1)N1CC[C@@H](Cn2c(-c3ccc(-c4ccc5[nH]ccc5c4)cc3Cl)n[nH]c2=O)C1]

A microwave vial was charged with 5-(4-bromo-2-chlorophenyl)-4-{[(3R)-1-(cyclopropylcarbonyl)-3-pyrrolidinyl]methyl}-2,4-dihydro-3H-1,2,4-triazol-3-one (150 mg, 0.352 mmol, >98% ee), 5-(4,4,5,5-tetramethyl-1,3,2-dioxaborolan-2-yl)-1H-indole (86 mg, 0.352 mmol), PdCl₂(dppf) (12.89 mg, 0.018 mmol), K₂CO₃ (122 mg, 0.881 mmol), 1,4-dioxane (3 mL), and water (1 mL). The reaction vessel was purged with N₂, sealed, and heated in a microwave reactor at 150° C. for 30 min. The reaction mixture was diluted with water, adjusted to pH ˜6.5, and extracted twice with CH₂Cl₂. The combined extracts were dried over Na₂SO₄, filtered, and concentrated under reduced pressure. The residue was purifed by silica gel chromatography (Analogix, 40 g column, 7% MeOH in EtOAc) followed by re-purification by reverse phase HPLC (10-90% CH₃CN/water+0.1% TFA). The appropriate fractions were concentrated to remove the CH₃CN, giving an aqueous suspension of product which was collected by filtration and dried to constant weight to afford the title compound (66 mg, 40.5%) as a white solid. MS (ES)⁺ m/e 462.2 [M+H]⁺. ¹H NMR (400 MHz, DMSO-d₆) δ ppm 12.08 (d, J=2.53 Hz, 1H), 11.27 (br. s., 1H), 7.99 (s, 1H), 7.96 (dd, J=4.67, 1.64 Hz, 1H), 7.83 (ddd, J=8.02, 6.13, 1.77 Hz, 1H), 7.62-7.74 (m, 1H), 7.49-7.56 (m, 2H), 7.43 (t, J=2.65 Hz, 1H), 6.49-6.56 (m, 1H), 3.63-3.10 (m, 6H), 2.25-2.49 (m, 1H), 1.71-1.96 (m, 1H), 1.35-1.68 (m, 2H), 0.56-0.71 (m, 4H).

### Example 19

### 5-[4-(1-benzofuran-5-yl)-2-chlorophenyl]-4-{[(3R)-1-(cyclopropylcarbonyl)-3-pyrrolidinyl]methyl}-2,4-dihydro-3H-1,2,4-triazol-3-one

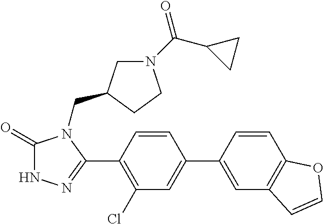 [SMILES: O=C(C1CC1)N1CC[C@@H](Cn2c(-c3ccc(-c4ccc5occc5c4)cc3Cl)n[nH]c2=O)C1]

A microwave vial was charged with 5-(4-bromo-2-chlorophenyl)-4-{[(3R)-1-(cyclopropylcarbonyl)-3-pyrrolidinyl]methyl}-2,4-dihydro-3H-1,2,4-triazol-3-one (150 mg, 0.352 mmol, >98% ee), 5-(4,4,5,5-tetramethyl-1,3,2-dioxaborolan-2-yl)-1-benzofuran (90 mg, 0.370 mmol), PdCl₂(dppf) (12.89 mg, 0.018 mmol), K₂CO₃ (122 mg, 0.881 mmol), 1,4-dioxane (3 mL), and water (1 mL). The reaction vessel was purged with N₂, sealed, and heated in a microwave reactor at 150° C. for 30 min. The reaction mixture was concentrated under reduced pressure and the residue was dissolved in DMSO, filtered through a syringe filter, and purified by reverse phase HPLC (10-95% CH₃CN/water+0.1% TFA). The appropriate fractions were concentrated under reduced pressure to remove a majority of the CH₃CN, leaving an aqueous mixture which was neutralized with saturated aq. NaHCO₃ and extracted twice with CH₂Cl₂. The organic extracts were dried over Na₂SO₄, filtered, concentrated, and dried to constant weight in a 50° C. vacuum oven to afford the title compound (99 mg, 60.7%) as a tan solid. MS (ES)⁺ m/e 463.0 [M+H]⁺. ¹H NMR (400 MHz, DMSO-d₆) δ ppm 12.11 (d, J=2.02 Hz, 1H), 8.06-8.15 (m, 2H), 8.02 (dd, J=5.31, 1.52 Hz, 1H), 7.82-7.91 (m, 1H), 7.67-7.81 (m, 3H), 7.05 (d, J=1.77 Hz, 1H), 3.63-2.88 (m, 6H), 2.23-2.48 (m, 1H), 1.70-1.97 (m, 1H), 1.35-1.70 (m, 2H), 0.55-0.73 (m, 4H).

### Example 20

### 5-(3-chloro-4′-fluoro-4-biphenylyl)-4-{[(3R)-1-(cyclopropylcarbonyl)-3-pyrrolidinyl]methyl}-2,4-dihydro-3H-1,2,4-triazol-3-one

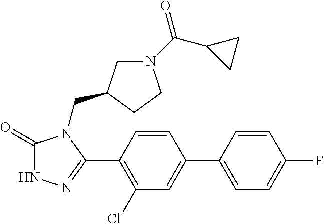 [SMILES: O=C(C1CC1)N1CC[C@@H](Cn2c(-c3ccc(-c4ccc(F)cc4)cc3Cl)n[nH]c2=O)C1]

A microwave vial was charged with 5-(4-bromo-2-chlorophenyl)-4-{[(3R)-1-(cyclopropylcarbonyl)-3-pyrrolidinyl]methyl}-2,4-dihydro-3H-1,2,4-triazol-3-one (150 mg, 0.352 mmol, >98% ee), (4-fluorophenyl)boronic acid (51.8 mg, 0.370 mmol), PdCl₂(dppf) (12.89 mg, 0.018 mmol), K₂CO₃ (122 mg, 0.881 mmol), 1,4-dioxane (3 mL), and water (1 mL). The reaction vessel was purged with N₂, sealed, and heated in a microwave reactor at 150° C. for 30 min. The reaction mixture was concentrated under reduced pressure and the residue was dissolved in DMSO, filtered through a syringe filter, and purified by reverse phase HPLC (10-95% CH₃CN/water+0.1% TFA). The appropriate fractions were concentrated under reduced pressure to remove a majority of the CH₃CN, leaving an aqueous mixture which was neutralized with saturated aq. NaHCO₃ and extracted twice with CH₂Cl₂. The organic extracts were dried over Na₂SO₄, filtered, concentrated, and dried to constant weight in a 50° C. vacuum oven to afford the title compound (115 mg, 74.0%) as an off-white solid. MS (ES)⁺ m/e 441.0 [M+H]⁺. ¹H NMR (400 MHz, DMSO-d₆) δ ppm 12.11 (d, J=1.77 Hz, 1H), 7.99 (dd, J=5.68, 1.39 Hz, 1H), 7.67-7.93 (m, 4H), 7.36 (t, J=8.72 Hz, 2H), 3.63-2.87 (m, 6H), 2.21-2.48 (m, 1H), 1.70-1.95 (m, 1H), 1.34-1.68 (m, 2H), 0.55-0.72 (m, 4H).

### Example 21

### 4-{[(3R)-1-(cyclopropylcarbonyl)-3-pyrrolidinyl]methyl}-5-[4-(1H-indol-6-yl)phenyl]-2,4-dihydro-3H-1,2,4-triazol-3-one

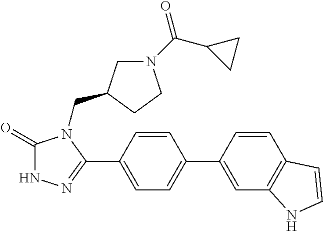 [SMILES: O=C(C1CC1)N1CC[C@@H](Cn2c(-c3ccc(-c4ccc5cc[nH]c5c4)cc3)n[nH]c2=O)C1]

### a) 2-[(4-bromophenyl)carbonyl]-N-{[(3S)-1-(cyclopropylcarbonyl)-3-pyrrolidinyl]methyl}hydrazinecarboxamide

{[(3S)-1-(cyclopropylcarbonyl)-3-pyrrolidinyl]methyl}amine (1.00 g, 5.94 mmol) and CDI (0.964 g, 5.94 mmol) were combined in dry CH₂Cl₂ (20 mL) and stirred at ambient temperature for 3 d. The reaction mixture was concentrated to dryness and the solvent replaced with dry THF (20 mL). The reaction mixture was treated with 4-bromobenzohydrazide (1.278 g, 5.94 mmol) at reflux for 3 h with stirring, during which time a heavy white precipitate formed requiring addition of THF (10 mL) to facilitate stirring. The reaction mixture was allowed to cool to ambient temperature and the solid was filtered off, washed with THF, and air dried to afford the title compound (1.89 g, 78%) as a white solid. LC-MS (ES⁻) m/z 407.12, 409.09 [M−1]. LC-MS (ES⁺) m/z 409.06, 411.08 [M+H]. ¹H NMR (400 MHz, DMSO-d₆) δ ppm 10.20-10.25 (m, 1H), 7.90 (d, J=7.73 Hz, 1H), 7.83 (d, J=8.61 Hz, 2H), 7.68-7.74 (m, 2H), 6.68-6.81 (m, 1H), 2.92-3.73 (m, 6H), 2.22-2.45 (m, 1H), 1.77-2.02 (m, 1H), 1.48-1.78 (m, 2H), 0.64-0.74 (m, 4H).

### b) 5-(4-bromophenyl)-4-{[(3R)-1-(cyclopropylcarbonyl)-3-pyrrolidinyl]methyl}-2,4-dihydro-3H-1,2,4-triazol-3-one

2-[(4-bromophenyl)carbonyl]-N-{[(3S)-1-(cyclopropylcarbonyl)-3-pyrrolidinyl]methyl}hydrazinecarboxamide (3.80 g, 9.28 mmol) was treated with 5% aq. K₂CO₃ (250 mL, 90 mmol) at reflux for 16 h. LCMS indicated complete conversion. The reaction mixture was extracted six times with EtOAc, at which point minimal product remained in the aqueous layer. The organic phases were combined, dried over MgSO₄, filtered, and concentrated to dryness to afford the title compound (2.95 g, 81% yield) as a white foam. LC-MS (ES⁻) m/z 389.25, 391.15 [M−1]. LC-MS (ES⁺) m/z 390.98, 392.99 [M+H]. ¹H NMR (400 MHz, DMSO-d₆) δ ppm 12.04 (s, 1H), 7.70-7.77 (m, 2H), 7.59-7.65 (m, 2H), 3.73-3.80 (m, 2H), 2.83-3.61 (m, 4H), 2.21-2.46 (m, 1H), 1.68-1.91 (m, 1H), 1.34-1.68 (m, 2H), 0.65 (m, 4H).

### c) 4-{[(3R)-1-(cyclopropylcarbonyl)-3-pyrrolidinyl]methyl}-5-[4-(1H-indol-6-yl)phenyl]-2,4-dihydro-3H-1,2,4-triazol-3-one

5-(4-bromophenyl)-4-{[(3R)-1-(cyclopropylcarbonyl)-3-pyrrolidinyl]methyl}-2,4-dihydro-3H-1,2,4-triazol-3-one (0.053 g, 0.135 mmol), 1H-indol-6-ylboronic acid (0.044 g, 0.271 mmol), K₃PO₄ (0.115 g, 0.542 mmol), and Pd(PPh₃)₄ (0.016 g, 0.014 mmol) were combined in EtOH (1.5 mL) and water (1.5 mL), purged with N₂, and irradiated in a microwave reactor for 45 min at 100° C. The reaction mixture was concentrated to a small volume, diluted with water, and extracted thrice with EtOAc. The organic phases were combined, dried over MgSO₄, filtered, and concentrated to dryness. The residue was purified on a 40 g silica gel column eluting with EtOAc followed by a linear gradient of 100% EtOAc to 10% MeOH/CH₂Cl₂ to afford the title compound contaminated with a major impurity. This material was repurified by reverse phase HPLC on a Phenomenex® Luna 5μ C-18 column (150×21.2 mm) eluting with 10 to 100% CH₃CN/H₂O (0.1% formic acid buffer). Appropriate fractions were combined and concentrated to a white solid. The residues was treated with saturated aq. NaHCO₃ and extracted four times with CH₂Cl₂. The organic phases were combined, dried over MgSO₄, filtered, and concentrated to dryness to afford the title compound (0.041 g, 70.8%) as a white foam. LC-MS (ES⁻) m/z 426.27 [M−1]. LC-MS (ES⁺) m/z 428.15 [M+H]. ¹H NMR (400 MHz, DMSO-d₆) δ ppm 11.98 (s, 1H), 11.23 (s, 1H), 7.79-7.85 (m, 2H), 7.68-7.75 (m, 3H), 7.63 (d, J=8.22 Hz, 1H), 7.40 (t, J=2.59 Hz, 1H), 7.36 (dt, J=8.25, 1.70 Hz, 1H), 6.45 (br. s., 1H), 2.28-3.61 (m, 7H), 1.70-1.93 (m, 1H), 1.37-1.66 (m, 2H), 0.56-0.67 (m, 4H).

### Example 22

### 5-[4-(1-benzofuran-5-yl)phenyl]-4-{[(3R)-1-(cyclopropylcarbonyl)-3-pyrrolidinyl]methyl}-2,4-dihydro-3H-1,2,4-triazol-3-one

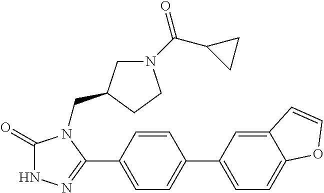 [SMILES: O=C(C1CC1)N1CC[C@@H](Cn2c(-c3ccc(-c4ccc5occc5c4)cc3)n[nH]c2=O)C1]

5-(4-Bromophenyl)-4-{[(3R)-1-(cyclopropylcarbonyl)-3-pyrrolidinyl]methyl}-2,4-dihydro-3H-1,2,4-triazol-3-one (0.090 g, 0.230 mmol), 5-(4,4,5,5-tetramethyl-1,3,2-dioxaborolan-2-yl)-1-benzofuran (0.112 g, 0.460 mmol), K₃PO₄ (0.195 g, 0.920 mmol), and Pd(PPh₃)₄ (0.027 g, 0.023 mmol) were combined in EtOH (2.5 mL) and water (2.5 mL), purged with N₂, and irradiated in a microwave reactor for 30 min at 100° C. The reaction mixture was concentrated to dryness, water was added, and the reaction mixture was extracted thrice with EtOAc. The organic phases were combined, dried over MgSO₄, filtered, and concentrated to dryness. The residue was purified on a 40 g silica gel column eluting with a linear gradient of 100% EtOAc to 10% MeOH/CH₂Cl₂ to afford the title compound (0.059 g, 59.9%) as a white foam. LC-MS (ES⁻) m/z 427.00 [M−1]. LC-MS (ES⁺) m/z 429.24 [M+H]. ¹H NMR (400 MHz, DMSO-d₆) δ ppm 12.02 (s, 1H), 8.06 (d, J=2.15 Hz, 1H), 8.02 (s, 1H), 7.83-7.89 (m, 2H), 7.66-7.79 (m, 4H), 7.01-7.06 (m, 1H), 3.78-3.89 (m, 2H), 3.43-3.64 (m, 2H), 2.31-3.31 (m, 3H), 1.71-1.94 (m, 1H), 1.40-1.68 (m, 2H), 0.64 (dd, J=6.80, 3.57 Hz, 4H).

### Example 23

### 4-{[(3R)-1-(cyclopropylcarbonyl)-3-pyrrolidinyl]methyl}-5-[4-(1H-indazol-6-yl)phenyl]-2,4-dihydro-3H-1,2,4-triazol-3-one

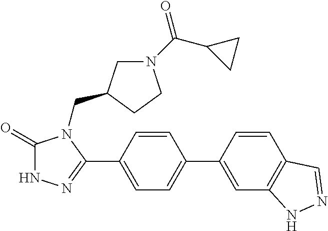 [SMILES: O=C(C1CC1)N1CC[C@@H](Cn2c(-c3ccc(-c4ccc5cn[nH]c5c4)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), 1,1-dimethylethyl 6-(4,4,5,5-tetramethyl-1,3,2-dioxaborolan-2-yl)-1H-indazole-1-carboxylate (0.132 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and heated in an oil bath at 100° C. for 4 h, after which time LCMS indicated incomplete conversion. The reaction mixture was then irradiated in a microwave reactor for 45 min at 100° C. at which point LCMS suggests modest improvement. The reaction mixture was concentrated to a small volume, diluted with water, and extracted thrice with EtOAc. The organic phases were combined, dried over MgSO₄, filtered, and concentrated to dryness. The residue was purified on a 40 g silica gel column eluting with 20 to 100% EtOAc/hexanes to remove a major impurity, followed by a linear gradient of 100% EtOAc to 10% MeOH/CH₂Cl₂ to afford the title compound (0.025 g, 30.4%) as a yellow foam. LC-MS (ES⁻) m/z 427.28 [M−1]. LC-MS (ES⁺) m/z 429.02 [M+H]. ¹H NMR (400 MHz, DMSO-d₆) δ ppm 13.21 (s, 1H), 12.02 (s, 1H), 8.12 (s, 1H), 7.85-7.94 (m, 3H), 7.82 (s, 1H), 7.74-7.81 (m, 2H), 7.49 (dt, J=8.43, 1.58 Hz, 1H), 3.79-3.89 (m, 2H), 2.90-3.64 (m, 4H), 2.30-2.54 (m, 1H), 1.72-1.95 (m, 1H), 1.39-1.69 (m, 2H), 0.64 (dd, J=6.82, 3.49 Hz, 4H).

### Example 24

### 4-{[(3R)-1-(cyclopropylcarbonyl)-3-pyrrolidinyl]methyl}-5-[4-(1H-indazol-5-yl)phenyl]-2,4-dihydro-3H-1,2,4-triazol-3-one

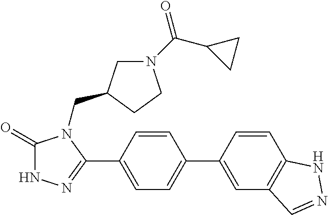 [SMILES: O=C(C1CC1)N1CC[C@@H](Cn2c(-c3ccc(-c4ccc5[nH]ncc5c4)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), 1,1-dimethylethyl 5-(4,4,5,5-tetramethyl-1,3,2-dioxaborolan-2-yl)-1H-indazole-1-carboxylate (0.132 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and heated in an oil bath at 100° C. for 4 h, after which time LCMS indicated incomplete conversion. The reaction mixture was then irradiated in a microwave reactor for 45 min at 100° C. at which point the LCMS appeared unchanged. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.030 g, 36.5%) as a yellow foam. LC-MS (ES⁻) m/z 427.24 [M−1]. LC-MS (ES⁺) m/z 429.06 [M+H]. ¹H NMR (400 MHz, DMSO-d₆) δ ppm 13.17 (s, 1H), 12.00 (s, 1H), 8.10-8.19 (m, 2H), 7.87 (dd, J=8.49, 2.04 Hz, 2H), 7.72-7.79 (m, 3H), 7.62-7.69 (m, 1H), 3.78-3.89 (m, 2H), 2.89-3.63 (m, 4H), 2.30-2.56 (m, 1H), 1.71-1.94 (m, 1H), 1.39-1.69 (m, 2H), 0.57-0.69 (m, 4H).

### Example 25

### 5-(4′-chloro-4-biphenylyl)-4-{[(3R)-1-(cyclopropylcarbonyl)-3-pyrrolidinyl]methyl}-2,4-dihydro-3H-1,2,4-triazol-3-one

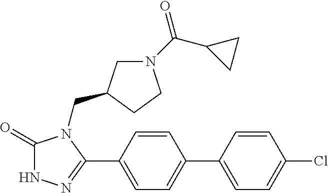 [SMILES: O=C(C1CC1)N1CC[C@@H](Cn2c(-c3ccc(-c4ccc(Cl)cc4)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), (4-chlorophenyl)boronic acid (0.060 g, 0.383 mmol), K₃PO₄ (0.163 g, 0.767 mmol), and Pd(PPh₃)₄ (0.022 g, 0.019 mmol) were combined in EtOH (2 mL), and water (2 mL), purged with N₂, and heated in an oil bath at 100° C. for 4 h, after which time LCMS indicated minimal conversion. The reaction mixture was then irradiated in a microwave reactor for 45 min at 120° C., after which time LCMS indicated complete conversion. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.0585 g, 72.2%) as a yellow foam. LC-MS (ES⁻) m/z 421.23 [M−1]. LC-MS (ES⁺) m/z 422.99 [M+H]. ¹H NMR (400 MHz, DMSO-d₆) δ ppm 12.02 (s, 1H), 7.72-7.88 (m, 6H), 7.56 (d, J=8.27 Hz, 2H), 3.78-3.87 (m, 2H), 2.88-3.64 (m, 4H), 2.27-2.54 (m, 1H), 1.70-1.93 (m, 1H), 1.40-1.67 (m, 2H), 0.59-0.67 (m, 4H).

### Example 26

### 4-{[(3R)-1-(cyclopropylcarbonyl)-3-pyrrolidinyl]methyl}-5-(4′-fluoro-4-biphenylyl)-2,4-dihydro-3H-1,2,4-triazol-3-one

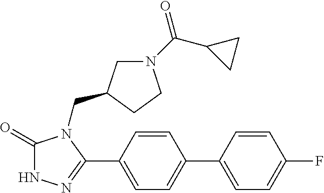 [SMILES: O=C(C1CC1)N1CC[C@@H](Cn2c(-c3ccc(-c4ccc(F)cc4)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), (4-fluorophenyl)boronic acid (0.054 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and heated in an oil bath at 100° C. for 4 h, after which time LCMS indicated incomplete conversion. The reaction mixture was then irradiated in a microwave reactor for 45 min at 120° C. after which LCMS indicated complete conversion. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.0558 g, 71.6%) as a yellow foam. LC-MS (ES⁻) m/z 405.28 [M−1]. LC-MS (ES⁺) m/z 407.05 [M+H]. ¹H NMR (400 MHz, DMSO-d₆) δ ppm 12.01 (s, 1H), 7.71-7.86 (m, 6H), 7.33 (t, J=8.65 Hz, 2H), 3.76-3.87 (m, 2H), 2.88-3.63 (m, 4H), 2.27-2.54 (m, 1H), 1.69-1.93 (m, 1H), 1.36-1.68 (m, 2H), 0.58-0.68 (m, 4H).

### Example 27

### 5-[2′-chloro-4′-(methyloxy)-4-biphenylyl]-4-{[(3R)-1-(cyclopropylcarbonyl)-3-pyrrolidinyl]methyl}-2,4-dihydro-3H-1,2,4-triazol-3-one

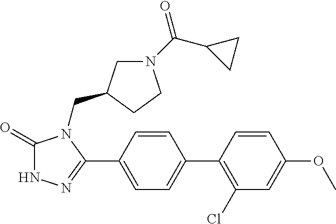 [SMILES: COc1ccc(-c2ccc(-c3n[nH]c(=O)n3C[C@@H]3CCN(C(=O)C4CC4)C3)cc2)c(Cl)c1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), [2-chloro-4-(methyloxy)phenyl]boronic acid (0.071 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and heated in an oil bath at 100° C. for 4 h, after which time LCMS indicated incomplete reaction. The reaction mixture was then irradiated in a microwave reactor for 45 min at 100° C., after which time the reaction appeared to be complete. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.065 g, 74.9%) as a yellow foam. LC-MS (ES⁻) m/z 421.23 [M−1]. LC-MS (ES⁺) m/z 422.99 [M+H]. ¹H NMR (400 MHz, DMSO-d₆) δ ppm 12.02 (s, 1H), 7.74 (dd, J=8.38, 5.27 Hz, 2H), 7.56 (d, J=7.52 Hz, 2H), 7.40 (dd, J=8.60, 4.30 Hz, 1H), 7.18 (d, J=2.58 Hz, 1H), 7.05 (ddd, J=8.62, 2.50, 1.24 Hz, 1H), 3.72-3.89 (m, 5H), 2.91-3.64 (m, 4H), 2.31-2.56 (m, 1H), 1.71-1.93 (m, 1H), 1.38-1.68 (m, 2H), 0.59-0.71 (m, 4H).

### Example 28

### 4-{[(3R)-1-(cyclopropylcarbonyl)-3-pyrrolidinyl]methyl}-5-[4-(1H-indol-5-yl)phenyl]-2,4-dihydro-3H-1,2,4-triazol-3-one

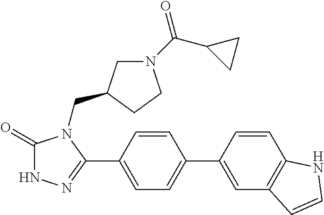 [SMILES: O=C(C1CC1)N1CC[C@@H](Cn2c(-c3ccc(-c4ccc5[nH]ccc5c4)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), 1H-indol-5-ylboronic acid (0.062 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and irradiated in a microwave reactor for 1 h at 120° C., after which time LCMS indicated complete consumption of the starting material. The reaction mixture was concentrated to a small volume, diluted with water, and extracted thrice with CH₂Cl₂. The organic phases were combined, dried over MgSO₄, filtered, and concentrated to dryness. The residue was purified by reverse phase HPLC on a SunFire Prep C-18 OBD 5μ 30×50 mm column eluted with 5 to 60% CH₃CN/H₂O (0.1% TFA buffer). Appropriate fractions were combined and concentrated to afford the title compound (0.0414 g, 50.5%) as a white solid. LC-MS (ES⁻) m/z 426.93 [M−1]. LC-MS (ES⁺) m/z 428.43 [M+H]. ¹H NMR (400 MHz, DMSO-d₆) δ ppm 11.98 (s, 1H), 11.20 (br. s., 1H), 7.91 (s, 1H), 7.79-7.87 (m, 2H), 7.67-7.76 (m, 2H), 7.44-7.53 (m, 2H), 7.40 (t, J=2.69 Hz, 1H), 6.51 (br. s., 1H), 3.77-3.88 (m, 2H), 2.90-3.62 (m, 4H), 2.31-2.54 (m, 1H), 1.71-1.95 (m, 1H), 1.41-1.68 (m, 2H), 0.57-0.69 (m, 4H).

### Example 29

### 5-[4-(1H-benzimidazol-5-yl)phenyl]-4-{[(3R)-1-(cyclopropylcarbonyl)-3-pyrrolidinyl]methyl}-2,4-dihydro-3H-1,2,4-triazol-3-one

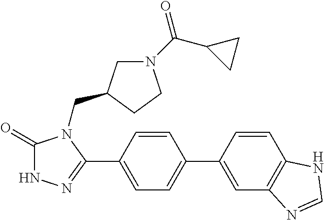 [SMILES: O=C(C1CC1)N1CC[C@@H](Cn2c(-c3ccc(-c4ccc5[nH]cnc5c4)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.0750 g, 0.192 mmol), 1,1-dimethylethyl 5-(4,4,5,5-tetramethyl-1,3,2-dioxaborolan-2-yl)-1H-benzimidazole-1-carboxylate (0.132 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and irradiated in a microwave reactor for 1 h at 120° C., after which time LCMS indicated complete consumption of the starting material. The reaction mixture was concentrated to a small volume, diluted with water, and extracted thrice with CH₂Cl₂. The organic phases were combined, dried over MgSO₄, filtered, and concentrated to dryness. The residue was triturated with MeOH and filtered to afford the title compound (0.0055 g, 6.7%) as a white precipitate. The filtrate was concentrated to dryness and was purified by reverse phase HPLC on a SunFire Prep C-18 OBD 5μ 30×50 mm column eluted with 5 to 35% CH₃CN/H₂O (0.1% TFA buffer). Appropriate fractions were combined and concentrated to afford a second batch of the title compound (0.0184 g, 22.4%) as a clear glass. LC-MS (ES⁻) m/z 427.71 [M−1]. LC-MS (ES⁺) m/z 429.36 [M+H]. ¹H NMR (400 MHz, DMSO-d₆) δ ppm 12.57 (d, J=16.12 Hz, 1H), 12.00 (br. s., 1H), 8.28 (br. s., 1H), 7.47-8.10 (m, 7H), 3.73-3.93 (m, 2H), 2.84-3.66 (m, 4H), 2.29-2.57 (m, 1H), 1.71-1.95 (m, 1H), 1.37-1.69 (m, 2H), 0.51-0.77 (m, 4H).

### Example 30

### 4-{[(3R)-1-(cyclopropylcarbonyl)-3-pyrrolidinyl]methyl}-5-(2′,4′-dichloro-4-biphenylyl)-2,4-dihydro-3H-1,2,4-triazol-3-one

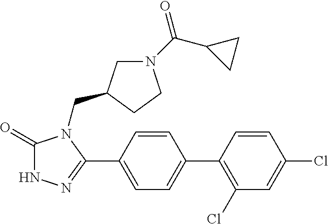 [SMILES: O=C(C1CC1)N1CC[C@@H](Cn2c(-c3ccc(-c4ccc(Cl)cc4Cl)cc3)n[nH]c2=O)C1]

5-(4-bromophenyl)-4-{[(3R)-1-(cyclopropylcarbonyl)-3-pyrrolidinyl]methyl}-2,4-dihydro-3H-1,2,4-triazol-3-one (0.075 g, 0.192 mmol), (2,4-dichlorophenyl)boronic acid (0.073 g, 0.383 mmol), K₃PO₄ (0.163 g, 0.767 mmol), and Pd(PPh₃)₄ (0.022 g, 0.019 mmol) were combined in EtOH (2 mL) and water (2 mL), purged with N₂, and irradiated in a microwave reactor for 3 h at 120° C., after which time LCMS indicated complete consumption of the starting material. The reaction mixture was concentrated to a small volume, diluted with water, and extracted thrice with EtOAc. The organic phases were combined, dried over MgSO₄, filtered, and concentrated to dryness. The residue was purified by reverse phase HPLC on a SunFire Prep C-18 OBD 5μ 30×50 mm column eluted with 5 to 70% CH₃CN/H₂O (0.1% TFA buffer). Appropriate fractions were combined and concentrated to afford the title compound (0.0445 g, 50.8%) as a white foam. LC-MS (ES⁻) m/z 455.10, 457.12 [M−1]. LC-MS (ES⁺) m/z 457.08, 459.08 [M+H]. ¹H NMR (400 MHz, DMSO-d₆) δ ppm 12.04 (s, 1H), 7.73-7.82 (m, 3H), 7.46-7.64 (m, 4H), 3.77-3.88 (m, 2H), 2.91-3.64 (m, 4H), 2.32-2.54 (m, 1H), 1.71-1.93 (m, 1H), 1.38-1.68 (m, 2H), 0.59-0.70 (m, 4H).

### Example 31

### 4-{[(3R)-1-(cyclopropylcarbonyl)-3-pyrrolidinyl]methyl}-5-[4-(1H-indol-6-yl)phenyl]-2-methyl-2,4-dihydro-3H-1,2,4-triazol-3-one

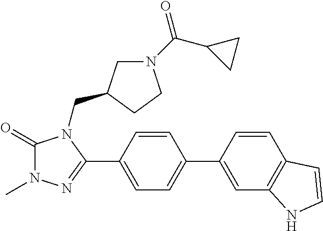 [SMILES: Cn1nc(-c2ccc(-c3ccc4cc[nH]c4c3)cc2)n(C[C@@H]2CCN(C(=O)C3CC3)C2)c1=O]

### a) 5-(4-bromophenyl)-4-{[(3R)-1-(cyclopropylcarbonyl)-3-pyrrolidinyl]methyl}-2-methyl-2,4-dihydro-3H-1,2,4-triazol-3-one

Iodomethane (0.084 mL, 1.341 mmol) was added to a solution of 5-(4-bromophenyl)-4-{[(3R)-1-(cyclopropylcarbonyl)-3-pyrrolidinyl]methyl}-2,4-dihydro-3H-1,2,4-triazol-3-one (0.477 g, 1.219 mmol) in CH₃CN (10 mL) with K₂CO₃ (0.337 g, 2.438 mmol) and stirred overnight at 60° C., after which time LCMS indicated ˜60% conversion. Additional iodomethane (0.360 mL, 5.76 mmol) was added to the reaction mixture and heating and stirring continued for 3 d, after which time LCMS indicated complete conversion. The reaction mixture was concentrated to dryness and water was added to the residue and extracted three times with EtOAc. The organic phases were combined, dried over MgSO₄, filtered, and concentrated to dryness. The residue was triturated with hexanes and filtered to afford the title compound (0.471 g, 95%) as a white solid. LC-MS (ES⁻) m/z 404.88 [M−1]. LC-MS (ES⁺) m/z 406.88 [M+H]. ¹H NMR (400 MHz, DMSO-d₆) δ ppm 7.71-7.77 (m, 2H), 7.59-7.65 (m, 2H), 3.73-3.84 (m, 2H), 3.41 (s, 3H), 2.84-3.62 (m, 4H), 2.24-2.53 (m, 1H), 1.70-1.91 (m, 1H), 1.35-1.68 (m, 2H), 0.60-0.69 (m, 4H).

### b) 4-{[(3R)-1-(cyclopropylcarbonyl)-3-pyrrolidinyl]methyl}-5-[4-(1H-indol-6-yl)phenyl]-2-methyl-2,4-dihydro-3H-1,2,4-triazol-3-one

5-(4-Bromophenyl)-4-{[(3R)-1-(cyclopropylcarbonyl)-3-pyrrolidinyl]methyl}-2-methyl-2,4-dihydro-3H-1,2,4-triazol-3-one (0.0785 g, 0.194 mmol), 1H-indol-6-ylboronic acid (0.062 g, 0.387 mmol), K₃PO₄ (0.164 g, 0.775 mmol), and Pd(PPh₃)₄ (0.022 g, 0.019 mmol) were combined in EtOH (2 mL) and water (2 mL), purged with N₂, and irradiated in a microwave reactor for 1 h at 110° C., after which time LCMS indicated complete conversion. The reaction mixture was filtered through Celite®, diluted with 100 mL EtOH, and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.077 g, 90%) as a tan foam. LC-MS (ES⁻) m/z 440.16 [M−1]. LC-MS (ES⁺) m/z 442.20 [M+H]. ¹H NMR (400 MHz, DMSO-d₆) δ ppm 11.24 (br. s., 1H), 7.81-7.88 (m, 2H), 7.70-7.77 (m, 3H), 7.65 (d, J=8.27 Hz, 1H), 7.35-7.45 (m, 2H), 6.47 (br. s., 1H), 3.81-3.91 (m, 2H), 3.44 (s, 3H), 2.91-3.64 (m, 4H), 2.34-2.58 (m, 1H), 1.72-1.95 (m, 1H), 1.40-1.68 (m, 2H), 0.59-0.69 (m, 4H).

### Example 32

### 5-[4-(1-benzofuran-5-yl)phenyl]-4-{[(3R)-1-(cyclopropylcarbonyl)-3-pyrrolidinyl]methyl}-2-methyl-2,4-dihydro-3H-1,2,4-triazol-3-one

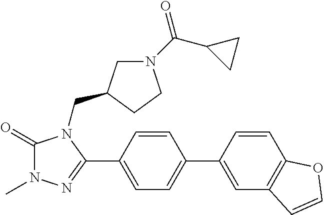 [SMILES: Cn1nc(-c2ccc(-c3ccc4occc4c3)cc2)n(C[C@@H]2CCN(C(=O)C3CC3)C2)c1=O]

5-(4-Bromophenyl)-4-{[(3R)-1-(cyclopropylcarbonyl)-3-pyrrolidinyl]methyl}-2-methyl-2,4-dihydro-3H-1,2,4-triazol-3-one (0.0785 g, 0.194 mmol), 5-(4,4,5,5-tetramethyl-1,3,2-dioxaborolan-2-yl)-1-benzofuran (0.062 g, 0.254 mmol), K₃PO₄ (0.164 g, 0.775 mmol), and Pd(PPh₃)₄ (0.022 g, 0.019 mmol) were combined in EtOH (2 mL) and water (2 mL), purged with N₂, and irradiated in a microwave reactor for 1 h at 110° C., after which time LCMS indicated complete conversion. The reaction mixture was filtered through Celite®, diluted with 100 mL EtOH, and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.079 g, 92%) as a white foam. LC-MS (ES⁺) m/z 443.19 [M+H]. ¹H NMR (400 MHz, DMSO-d₆) δ ppm 8.06 (d, J=2.26 Hz, 1H), 8.02 (s, 1H), 7.84-7.90 (m, 2H), 7.66-7.79 (m, 4H), 7.02-7.06 (m, 1H), 3.82-3.90 (m, 2H), 3.44 (s, 3H), 2.89-3.64 (m, 4H), 2.33-2.55 (m, 1H), 1.72-1.94 (m, 1H), 1.39-1.68 (m, 2H), 0.59-0.68 (m, 4H).

### Example 33

### 4-{[(3R)-1-(cyclopropylcarbonyl)-3-pyrrolidinyl]methyl}-5-[4-(1H-indol-5-yl)phenyl]-2-methyl-2,4-dihydro-3H-1,2,4-triazol-3-one

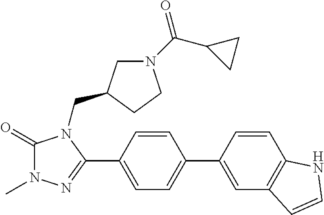 [SMILES: Cn1nc(-c2ccc(-c3ccc4[nH]ccc4c3)cc2)n(C[C@@H]2CCN(C(=O)C3CC3)C2)c1=O]

5-(4-Bromophenyl)-4-{[(3R)-1-(cyclopropylcarbonyl)-3-pyrrolidinyl]methyl}-2-methyl-2,4-dihydro-3H-1,2,4-triazol-3-one (0.0785 g, 0.194 mmol), 1H-indol-5-ylboronic acid (0.062 g, 0.387 mmol), K₃PO₄ (0.164 g, 0.775 mmol), and Pd(PPh₃)₄ (0.022 g, 0.019 mmol) were combined in EtOH (2 mL) and water (2 mL), purged with N₂, and irradiated in a microwave reactor for 1 h at 110° C., after which time LCMS indicated complete conversion. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.074 g, 87%) as a tan foam. LC-MS (ES⁻) m/z 440.25 [M−1]. LC-MS (ES⁺) m/z 442.21 [M+H]. ¹H NMR (400 MHz, DMSO-d₆) δ ppm 11.22 (br. s., 1H), 7.92 (s, 1H), 7.80-7.87 (m, 2H), 7.72 (dd, J=8.42, 3.23 Hz, 2H), 7.44-7.53 (m, 2H), 7.40 (t, J=2.69 Hz, 1H), 6.51 (br. s., 1H), 3.80-3.91 (m, 2H), 3.43 (s, 3H), 2.90-3.64 (m, 4H), 2.34-2.56 (m, 1H), 1.71-1.94 (m, 1H), 1.39-1.68 (m, 2H), 0.58-0.69 (m, 4H).

### Example 34

### 4-{[(3R)-1-(cyclopropylcarbonyl)-3-pyrrolidinyl]methyl}-5-[4-(1H-indazol-6-yl)phenyl]-2-methyl-2,4-dihydro-3H-1,2,4-triazol-3-one

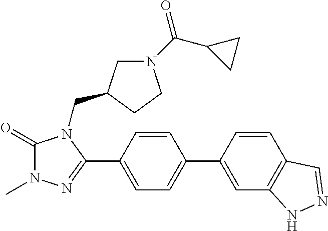 [SMILES: Cn1nc(-c2ccc(-c3ccc4cn[nH]c4c3)cc2)n(C[C@@H]2CCN(C(=O)C3CC3)C2)c1=O]

5-(4-Bromophenyl)-4-{[(3R)-1-(cyclopropylcarbonyl)-3-pyrrolidinyl]methyl}-2-methyl-2,4-dihydro-3H-1,2,4-triazol-3-one (0.0785 g, 0.194 mmol), 1,1-dimethylethyl 6-(4,4,5,5-tetramethyl-1,3,2-dioxaborolan-2-yl)-1H-indazole-1-carboxylate (0.080 g, 0.232 mmol), K₃PO₄ (0.164 g, 0.775 mmol), and Pd(PPh₃)₄ (0.022 g, 0.019 mmol) were combined in EtOH (2 mL) and water (2 mL), purged with N₂, and irradiated in a microwave reactor for 1 h at 110° C., after which time LCMS indicated complete conversion. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.059 g, 68.8%) as a tan foam. LC-MS (ES⁻) m/z 441.53 [M−1]. LC-MS (ES⁺) m/z 442.81 [M+H]. ¹H NMR (400 MHz, DMSO-d₆) δ ppm 13.21 (s, 1H), 8.12 (s, 1H), 7.75-7.94 (m, 6H), 7.50 (dt, J=8.49, 1.67 Hz, 1H), 3.82-3.93 (m, 2H), 3.44 (s, 3H), 2.89-3.65 (m, 4H), 2.34-2.58 (m, 1H), 1.72-1.95 (m, 1H), 1.39-1.68 (m, 2H), 0.58-0.68 (m, 4H).

### Example 35

### 5-[2′-chloro-4′-(methyloxy)-4-biphenylyl]-4-{[(3R)-1-(cyclopropylcarbonyl)-3-pyrrolidinyl]methyl}-2-methyl-2,4-dihydro-3H-1,2,4-triazol-3-one

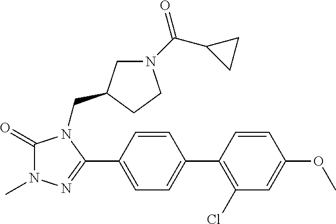 [SMILES: COc1ccc(-c2ccc(-c3nn(C)c(=O)n3C[C@@H]3CCN(C(=O)C4CC4)C3)cc2)c(Cl)c1]

5-(4-Bromophenyl)-4-{[(3R)-1-(cyclopropylcarbonyl)-3-pyrrolidinyl]methyl}-2-methyl-2,4-dihydro-3H-1,2,4-triazol-3-one (0.0785 g, 0.194 mmol), [2-chloro-4-(methyloxy)phenyl]boronic acid (0.072 g, 0.387 mmol), K₃PO₄ (0.164 g, 0.775 mmol), and Pd(PPh₃)₄ (0.022 g, 0.019 mmol) were combined in EtOH (2 mL) and water (2 mL), purged with N₂, and irradiated in a microwave reactor for 1 h at 110° C., after which time LCMS indicated complete conversion. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.088 g, 97%) as a tan foam. LC-MS (ES⁺) m/z 467.16, 469.14 [M+H]. ¹H NMR (400 MHz, DMSO-d₆) δ ppm 7.71-7.77 (m, 2H), 7.57 (d, J=7.54 Hz, 2H), 7.40 (dd, J=8.61, 4.40 Hz, 1H), 7.18 (d, J=2.54 Hz, 1H), 7.05 (ddd, J=8.59, 2.52, 1.22 Hz, 1H), 3.83 (s, 3H), 3.79-3.90 (m, 2H), 3.43 (s, 3H), 2.90-3.67 (m, 4H), 2.34-2.56 (m, 1H), 1.72-1.94 (m, 1H), 1.39-1.69 (m, 2H), 0.60-0.69 (m, 4H).

### Example 36

### 4-{[(3R)-1-(cyclopropylcarbonyl)-3-pyrrolidinyl]methyl}-5-(4′-fluoro-4-biphenylyl)-2-methyl-2,4-dihydro-3H-1,2,4-triazol-3-one

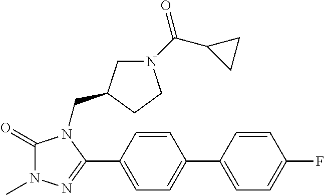 [SMILES: Cn1nc(-c2ccc(-c3ccc(F)cc3)cc2)n(C[C@@H]2CCN(C(=O)C3CC3)C2)c1=O]

5-(4-Bromophenyl)-4-{[(3R)-1-(cyclopropylcarbonyl)-3-pyrrolidinyl]methyl}-2-methyl-2,4-dihydro-3H-1,2,4-triazol-3-one (0.0785 g, 0.194 mmol), (4-fluorophenyl)boronic acid (0.054 g, 0.387 mmol), K₃PO₄ (0.164 g, 0.775 mmol), and Pd(PPh₃)₄ (0.022 g, 0.019 mmol) were combined in EtOH (2 mL) and water (2 mL), purged with N₂, and irradiated in a microwave reactor for 1 h at 110° C., after which time LCMS indicated complete conversion. The reaction mixture was diluted with 100 mL EtOH and concentrated to dryness. The residue was triturated with a small amount of CH₂Cl₂ and the triturate was loaded directly onto a 40 g silica gel column and eluted with a linear gradient of 100% EtOAc to 10% MeOH/CH₂Cl₂ to afford the title compound (0.085 g, quantitative yield) as a tan foam. LC-MS (ES⁺) m/z 421.20 [M+H]. ¹H NMR (400 MHz, DMSO-d₆) δ ppm 7.77-7.86 (m, 4H), 7.72-7.77 (m, 2H), 7.34 (m, 2H), 3.79-3.90 (m, 2H), 3.43 (s, 3H), 3.05-3.64 (m, 4H), 2.31-2.55 (m, 1H), 1.70-1.94 (m, 1H), 1.39-1.68 (m, 2H), 0.59-0.68 (m, 4H).

### Example 37

### 4-{[(3R)-1-(cyclopropylcarbonyl)-3-pyrrolidinyl]methyl}-5-(4′-fluoro-4-biphenylyl)-2-(2-hydroxy-2-methylpropyl)-2,4-dihydro-3H-1,2,4-triazol-3-one

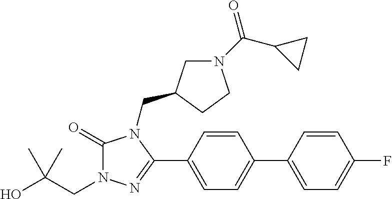 [SMILES: CC(C)(O)Cn1nc(-c2ccc(-c3ccc(F)cc3)cc2)n(C[C@@H]2CCN(C(=O)C3CC3)C2)c1=O]

4-{[(3R)-1-(Cyclopropylcarbonyl)-3-pyrrolidinyl]methyl}-5-(4′-fluoro-4-biphenylyl)-2,4-dihydro-3H-1,2,4-triazol-3-one (0.085 g, 0.209 mmol) and K₂CO₃ (0.104 g, 0.753 mmol) were combined in 2,2-dimethyloxirane (5 mL, 69.3 mmol) in a sealed tube and heated at 70° C. for 24 h, after which time LCMS indicated ˜50% conversion. The reaction mixture was returned to heating in a sealed tube at 80° C. for 24 h, after which time LCMS indicated ˜95% conversion. The reaction mixture was partitioned between EtOAc and water. The organic phase was isolated, dried over MgSO₄, filtered, and concentrated to dryness. The residue was purified on a 40 g silica gel column eluting with a linear gradient of 100% EtOAc to 10% MeOH/CH₂Cl₂ to afford the title compound (0.084 g, 84%) as a white foam. LC-MS (ES⁺) m/z 479.17 [M+H]. ¹H NMR (400 MHz, DMSO-d₆) δ ppm 7.72-7.87 (m, 6H), 7.34 (m, 2H), 4.66 (d, J=4.73 Hz, 1H), 3.83-3.92 (m, 2H), 3.68-3.73 (m, 2H), 2.84-3.62 (m, 4H), 2.33-2.57 (m, 1H), 1.69-1.94 (m, 1H), 1.36-1.68 (m, 2H), 1.16 (d, J=1.50 Hz, 6H), 0.58-0.68 (m, 4H).

### Example 38

### 2-acetyl-4-{[(3R)-1-(cyclopropylcarbonyl)-3-pyrrolidinyl]methyl}-5-(4′-fluoro-4-biphenylyl)-2,4-dihydro-3H-1,2,4-triazol-3-one

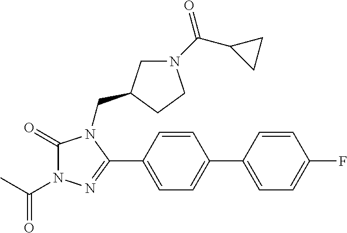 [SMILES: CC(=O)n1nc(-c2ccc(-c3ccc(F)cc3)cc2)n(C[C@@H]2CCN(C(=O)C3CC3)C2)c1=O]

4-{[(3R)-1-(Cyclopropylcarbonyl)-3-pyrrolidinyl]methyl}-5-(4′-fluoro-4-biphenylyl)-2,4-dihydro-3H-1,2,4-triazol-3-one (0.092 g, 0.226 mmol) was heated at 110° C. in acetic anhydride (5 mL, 53.0 mmol) for 1 h, after which time LCMS indicated complete conversion. The reaction mixture was concentrated under high vacuum and partitioned between EtOAc and water. The organic phase was isolated, dried over MgSO₄, filtered, and concentrated to dryness. The residue was purified on a 40 g silica gel column eluting with a linear gradient of 100% EtOAc to 5% MeOH/CH₂Cl₂ to afford the title compound (0.043 g, 42.4%). LC-MS (ES⁺) m/z 449.13 [M+H]. ¹H NMR (400 MHz, DMSO-d₆) δ ppm 7.76-7.94 (m, 6H), 7.35 (m, 2H), 3.79-3.88 (m, 2H), 2.87-3.68 (m, 4H), 2.55 (d, J=1.61 Hz, 3H), 2.33-2.53 (m, 1H), 1.73-1.97 (m, 1H), 1.39-1.67 (m, 2H), 0.58-0.69 (m, 4H).

### Example 39

### 2-(cyclopropylcarbonyl)-4-{[(3R)-1-(cyclopropylcarbonyl)-3-pyrrolidinyl]methyl}-5-(4′-fluoro-4-biphenylyl)-2,4-dihydro-3H-1,2,4-triazol-3-one

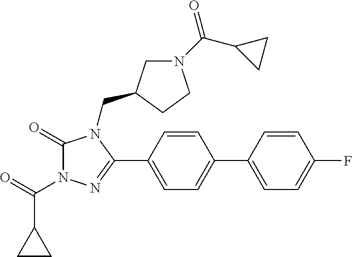 [SMILES: O=C(C1CC1)N1CC[C@@H](Cn2c(-c3ccc(-c4ccc(F)cc4)cc3)nn(C(=O)C3CC3)c2=O)C1]

4-{[(3R)-1-(Cyclopropylcarbonyl)-3-pyrrolidinyl]methyl}-5-(4′-fluoro-4-biphenylyl)-2,4-dihydro-3H-1,2,4-triazol-3-one (0.080 g, 0.197 mmol) was dissolved in DMF (0.5 mL) and treated with K₂CO₃ (0.054 g, 0.394 mmol) and cyclopropanecarbonyl chloride (0.021 g, 0.197 mmol) at 85° C. for 3 h, after which time LCMS indicated no reaction. Additional cyclopropanecarbonyl chloride (0.320 g, 3.06 mmol) was added to the reaction mixture and the mixture was stirred for three d at 85° C., after which time LCMS indicated ˜75% conversion. The reaction mixture was partitioned between EtOAc and water, and the organic phase was isolated, washed with water, dried over MgSO₄, filtered, and concentrated to dryness. The residue was purified on a 40 g silica gel column eluting with 40 to 100% EtOAc to afford the title compound (0.0378 g, 40.5%) as a white foam. LC-MS (ES⁺) m/z 475.10 [M+H]. ¹H NMR (400 MHz, DMSO-d₆) δ ppm 7.86-7.91 (m, 2H), 7.79-7.86 (m, 4H), 7.35 (m, 2H), 2.34-3.89 (m, 8H), 1.76-1.98 (m, 1H), 1.41-1.68 (m, 2H), 1.02-1.14 (m, 4H), 0.59-0.69 (m, 4H).

### Example 40

### 4-{[(3R)-1-(cyclopropylcarbonyl)-3-pyrrolidinyl]methyl}-N-ethyl-3-(4′-fluoro-4-biphenylyl)-5-oxo-4,5-dihydro-1H-1,2,4-triazole-1-carboxamide

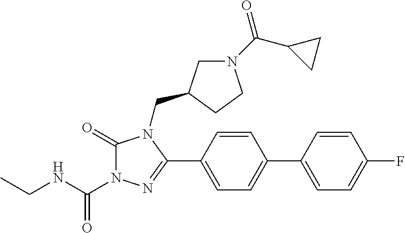 [SMILES: CCNC(=O)n1nc(-c2ccc(-c3ccc(F)cc3)cc2)n(C[C@@H]2CCN(C(=O)C3CC3)C2)c1=O]

To a suspension of 4-{[(3R)-1-(cyclopropylcarbonyl)-3-pyrrolidinyl]methyl}-5-(4′-fluoro-4-biphenylyl)-2,4-dihydro-3H-1,2,4-triazol-3-one (0.080 g, 0.197 mmol) in CH₃CN (3 mL) was added DBU (5.93 μl, 0.039 mmol) and isocyanatoethane (0.015 g, 0.217 mmol), and the reaction mixture was stirred at 25° C. for 16 h, after which time LCMS indicated complete conversion to the desired product. The reaction mixture was concentrated to dryness and purified on a 40 g silica gel column eluting with EtOAc followed by 100% EtOAc to 10% MeOH/CH₂Cl₂ to afford the desired compound contaminated with DBU. This material was partitioned between CH₂Cl₂ and 1 N HCl. The organic phase was isolated, dried over MgSO₄, filtered, and concentrated to dryness to afford the title compound (0.0721 g, 77%) as a clear oil. LC-MS (ES⁺) m/z 478.41 [M+H]. ¹H NMR (400 MHz, DMSO-d₆) δ ppm 8.16-8.23 (m, 1H), 7.86-7.91 (m, 2H), 7.78-7.85 (m, 4H), 7.35 (t, J=8.70 Hz, 2H), 2.89-3.90 (m, 8H), 2.34-2.54 (m, 1H), 1.75-1.97 (m, 1H), 1.43-1.69 (m, 2H), 1.14 (t, J=7.15 Hz, 3H), 0.59-0.69 (m, 4H).

### Example 41

### 4-{[(3R)-1-(cyclopropylcarbonyl)-3-pyrrolidinyl]methyl}-5-(4′-fluoro-4-biphenylyl)-2-[2-oxo-2-(1-pyrrolidinyl)ethyl]-2,4-dihydro-3H-1,2,4-triazol-3-one

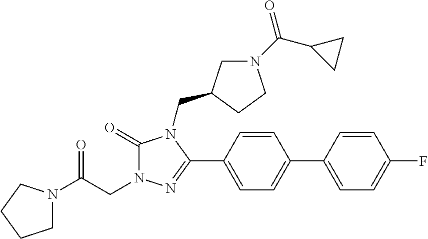 [SMILES: O=C(Cn1nc(-c2ccc(-c3ccc(F)cc3)cc2)n(C[C@@H]2CCN(C(=O)C3CC3)C2)c1=O)N1CCCC1]

4-{[(3R)-1-(Cyclopropylcarbonyl)-3-pyrrolidinyl]methyl}-5-(4′-fluoro-4-biphenylyl)-2,4-dihydro-3H-1,2,4-triazol-3-one (0.080 g, 0.197 mmol) was combined with 1-(chloroacetyl)pyrrolidine (0.032 g, 0.217 mmol) and K₂CO₃ (0.054 g, 0.394 mmol) in DMF (1 mL) and stirred at 80° C. for 16 h, after which time LCMS indicated complete conversion. The reaction mixture was diluted with EtOAc, washed twice with water and once with brine, dried over MgSO₄, filtered, and concentrated to dryness. The residue was purified on a 40 g silica gel column eluting with a linear gradient of 100% EtOAc to 10% MeOH/CH₂Cl₂ to afford the title compound (0.0817 g, 80%) as a white foam. LC-MS (ES⁺) m/z 518.22 [M+H]. ¹H NMR (400 MHz, DMSO-d₆) δ ppm 7.73-7.88 (m, 6H), 7.33 (m, 2H), 4.67 (s, 2H), 3.82-3.97 (m, 2H), 2.89-3.64 (m, 8H), 2.28-2.52 (m, 1H), 1.72-1.97 (m, 5H), 1.40-1.68 (m, 2H), 0.58-0.70 (m, 4H).

### Example 42

### 4-{[(3R)-1-(cyclopropylcarbonyl)-3-pyrrolidinyl]methyl}-5-(4′-fluoro-4-biphenylyl)-2-(phenylmethyl)-2,4-dihydro-3H-1,2,4-triazol-3-one

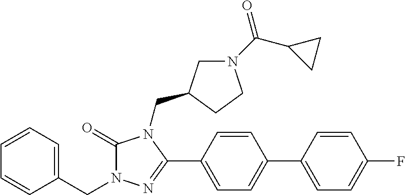 [SMILES: O=C(C1CC1)N1CC[C@@H](Cn2c(-c3ccc(-c4ccc(F)cc4)cc3)nn(Cc3ccccc3)c2=O)C1]

4-{[(3R)-1-(Cyclopropylcarbonyl)-3-pyrrolidinyl]methyl}-5-(4′-fluoro-4-biphenylyl)-2,4-dihydro-3H-1,2,4-triazol-3-one (0.080 g, 0.197 mmol) was combined with (bromomethyl)benzene (0.037 g, 0.217 mmol) and K₂CO₃ (0.054 g, 0.394 mmol) in DMF (1 mL) and stirred at 80° C. for 16 h, after which time LCMS indicated ˜50% conversion. Additional (bromomethyl)benzene (0.034 g, 0.197 mmol) was added to the reaction mixture and stirring was continued at 80° C. for 16 h. The reaction mixture was diluted with EtOAc, washed twice with water and once with brine, dried over MgSO₄, filtered, and concentrated to dryness. The residue was purified on a 40 g silica gel column eluting with a linear gradient of 100% EtOAc to 10% MeOH/CH₂Cl₂ to afford the title compound (0.0695 g, 71.1%) as a white foam. LC-MS (ES⁺) m/z 497.39 [M+H]. ¹H NMR (400 MHz, DMSO-d₆) δ ppm 7.70-7.86 (m, 6H), 7.26-7.41 (m, 7H), 5.01 (s, 2H), 3.84-3.94 (m, 2H), 2.89-3.63 (m, 4H), 2.34-2.56 (m, 1H), 1.71-1.95 (m, 1H), 1.40-1.68 (m, 2H), 0.59-0.68 (m, 4H).

### Example 43

### 4-{[(3R)-1-(cyclopropylcarbonyl)-3-pyrrolidinyl]methyl}-5-(4′-fluoro-4-biphenylyl)-2-[2-(1-pyrrolidinyl)ethyl]-2,4-dihydro-3H-1,2,4-triazol-3-one

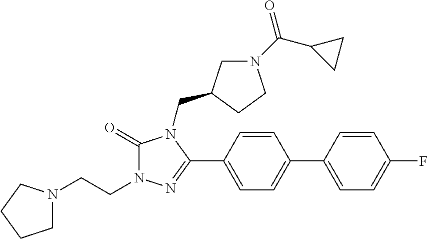 [SMILES: O=C(C1CC1)N1CC[C@@H](Cn2c(-c3ccc(-c4ccc(F)cc4)cc3)nn(CCN3CCCC3)c2=O)C1]

PPh₃ (0.052 g, 0.197 mmol) was dissolved in THF (1 mL), cooled to 0° C., and treated with DIAD (0.038 mL, 0.197 mmol) for 10 min. 2-(1-Pyrrolidinyl)ethanol (0.023 g, 0.197 mmol) and 4-{[(3R)-1-(cyclopropylcarbonyl)-3-pyrrolidinyl]methyl}-5-(4′-fluoro-4-biphenylyl)-2,4-dihydro-3H-1,2,4-triazol-3-one (0.080 g, 0.197 mmol) were added successively to the reaction mixture and stirred at 0° C. for 2 h and then allowed to warm to ambient temperature. The mixture was stirred overnight, after which time LCMS indicated ˜25% conversion. In a separate flask, PPh₃ (0.103 g, 0.394 mmol) was dissolved in THF (3 mL), cooled to 0° C. and treated with DIAD (0.038 mL, 0.197 mmol) for 15 min. To this reaction mixture was added 2-(1-pyrrolidinyl)ethanol (0.045 g, 0.394 mmol) and stirred for 5 min. The original reaction mixture was then added dropwise to the new solution using a small amount of THF to facilitate the transfer. The reaction mixture was stirred at 0° C. for 2 h and then allowed to warm to ambient temperature. The mixture was stirred overnight, after which time LCMS indicated ˜60% conversion. The reaction mixture was concentrated to dryness and partitioned between CH₂Cl₂ and saturated aq. NaHCO₃. The organic phase was isolated, dried over MgSO₄, filtered, and concentrated to dryness. The residue was purified by mass-directed HPLC on a SunFire prep C-18 column eluted with 30 to 85% CH₃CN/H₂O (0.1% formic acid buffer). Appropriate fractions were combined and concentrated to dryness. The residue was partitioned between CH₂Cl₂ and saturated aq. NaHCO₃. The organic phase was isolated, dried over MgSO₄, filtered, and concentrated to dryness to afford the title compound (0.0353 g, 35.6%) as a white foam. LC-MS (ES⁺) m/z 504.47 [M+H].

### Example 44

### 4-{[(3R)-1-(cyclopropylcarbonyl)-3-pyrrolidinyl]methyl}-5-(2′,3,4′-trifluoro-4-biphenylyl)-2,4-dihydro-3H-1,2,4-triazol-3-one

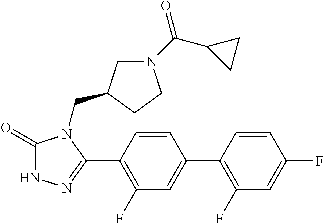 [SMILES: O=C(C1CC1)N1CC[C@@H](Cn2c(-c3ccc(-c4ccc(F)cc4F)cc3F)n[nH]c2=O)C1]

5-(4-Bromo-2-fluorophenyl)-4-{[(3R)-1-(cyclopropylcarbonyl)-3-pyrrolidinyl]methyl}-2,4-dihydro-3H-1,2,4-triazol-3-one (100 mg, 0.244 mmol), 2,4-difluorophenylboronic acid (40 mg, 0.253 mmol), PdCl₂(dppf) (20 mg, 0.024 mmol), 1,4-dioxane (2 mL), and 2 M aq. K₂CO₃ (1 mL) were placed in a 5 mL microwave vial. The vial was sealed and heated to 110° C. After 1 h, LCMS revealed that the reaction was complete. The reaction mixture was cooled to room temperature, neutralized with 6 N aq. HCl and diluted with 20 mL EtOAc. The reaction mixture was washed with water. The organic layer was separated, dried over Na₂SO₄, filtered, and concentrated to dryness. The crude material was taken up in 2 mL DMSO and purified by reverse phase HPLC, eluting with 10-70% CH₃CN/H₂O (0.1% NH₄OH). The desired fractions were collected and concentrated to dryness to afford the title compound (55 mg, 50.4%). MS (ES)⁺ m/e 443.2 [M+H]⁺.

### Example 45

### 4-{[(3R)-1-(cyclopropylcarbonyl)-3-pyrrolidinyl]methyl}-5-[3-fluoro-4′-(methyloxy)-4-biphenylyl]-2,4-dihydro-3H-1,2,4-triazol-3-one

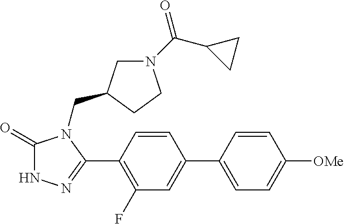 [SMILES: COc1ccc(-c2ccc(-c3n[nH]c(=O)n3C[C@@H]3CCN(C(=O)C4CC4)C3)c(F)c2)cc1]

The procedure described for Example 44 was used, replacing 2,4-difluorophenylboronic acid with 4-methoxyphenylboronic acid (1.077 equiv.), to afford the title compound (39 mg, 36.2%). MS (ES)⁺ m/e 437.3 [M+H]⁺.

### Example 46

### 4′-(4-{[(3R)-1-(cyclopropylcarbonyl)-3-pyrrolidinyl]methyl}-5-oxo-4,5-dihydro-1H-1,2,4-triazol-3-yl)-3′-fluoro-4-biphenylcarbonitrile

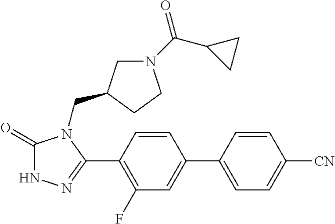 [SMILES: N#Cc1ccc(-c2ccc(-c3n[nH]c(=O)n3C[C@@H]3CCN(C(=O)C4CC4)C3)c(F)c2)cc1]

The procedure described for Example 44 was used, replacing 2,4-difluorophenylboronic acid with 4-cyanophenylboronic acid (1.114 equiv.), to afford the title compound (50 mg, 47%). MS (ES)⁺ m/e 432.2 [M+H]⁺.

### Example 47

### 5-(4′-chloro-3-fluoro-4-biphenylyl)-4-{[(3R)-1-(cyclopropylcarbonyl)-3-pyrrolidinyl]methyl}-2,4-dihydro-3H-1,2,4-triazol-3-one

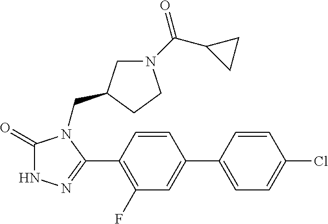 [SMILES: O=C(C1CC1)N1CC[C@@H](Cn2c(-c3ccc(-c4ccc(Cl)cc4)cc3F)n[nH]c2=O)C1]

The procedure described for Example 44 was used, replacing 2,4-difluorophenylboronic acid with 4-chlorophenylboronic acid (1.115 equiv.), to afford the title compound (41 mg, 38.1%). MS (ES)⁺ m/e 441.2 [M+H]⁺.

### Example 48

### 4-{[(3R)-1-(cyclopropylcarbonyl)-3-pyrrolidinyl]methyl}-5-[3′-(methyloxy)-4-biphenylyl]-2,4-dihydro-3H-1,2,4-triazol-3-one

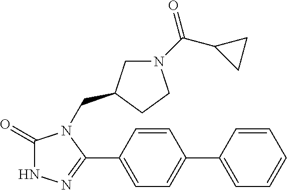 [SMILES: O=C(C1CC1)N1CC[C@@H](Cn2c(-c3ccc(-c4ccccc4)cc3)n[nH]c2=O)C1]

### a) 1,1-dimethylethyl(3S)-3-{[({2-[(4-bromophenyl)carbonyl]hydrazino}carbonyl)amino]methyl}-1-pyrrolidinecarboxylate

To a round bottom flask were added 1-dimethylethyl(3S)-3-(aminomethyl)-1-pyrrolidinecarboxylate (8.1 g, 40.4 mmol, 80% e.e), CDI (6.56 g, 40.4 mmol), and dry CH₂Cl₂ (90 mL). The mixture was purged with N₂ and stirred at room temperature for 16 h. LCMS analysis indicated desired intermediate formation. The solution was concentrated and 4-bromobenzohydrazide (9.0 g, 41.9 mmol) was added followed by THF (100 mL). The resulting mixture was refluxed for 6 h. LCMS analysis indicated desired product, and the solution was concentrated to give an off-white foam. The residue was purified by silica gel chromatography (0-20% i-PrOH+0.15% NH₄OH in CH₂Cl₂+0.15% NH₄OH). The desired fractions were collected and concentrated to afford the title compound (11 g, 62%) as a white foam. MS (ES)⁺ m/e 441.0, 443.1 [M+H]⁺.

### b) 5-(4-bromophenyl)-4-[(3R)-3-pyrrolidinylmethyl]-2,4-dihydro-3H-1,2,4-triazol-3-one

To a round bottom flask were added 1,1-dimethylethyl(3S)-3-{[({2-[(4-bromophenyl)carbonyl]hydrazino}carbonyl)amino]methyl}-1-pyrrolidinecarboxylate (8.6 g, 19.49 mmol), K₂CO₃ (13.47 g, 97 mmol), and water (250 mL). The resulting suspension was refluxed at 130° C. for 20 h. LCMS analysis indicated consumption of the starting material and desired product formation. The mixture was neutralized with 6 N HCl and concentrated to afford the title compound. MS (ES)⁺ m/e 323.0, 325.1 [M+H]⁺.

### c) 5-(4-bromophenyl)-4-{[(3R)-1-(cyclopropylcarbonyl)-3-pyrrolidinyl]methyl}-2,4-dihydro-3H-1,2,4-triazol-3-one

To a round bottom flask were added 5-(4-bromophenyl)-4-[(3R)-3-pyrrolidinylmethyl]-2,4-dihydro-3H-1,2,4-triazol-3-one (9 g, 27.8 mmol) and CH₂Cl₂ (99 mL) under N₂ and the mixture was cooled to −78° C. Cyclopropyl carbonyl chloride (2.60 mL, 28.4 mmol) and DIPEA (14.59 mL, 84 mmol) were added. The reaction mixture was allowed to warm room temperature and stirred for 30 min. LCMS analysis shows product and starting material. The mixture was recooled to −78° C. and another equivalent of cyclopropyl carbonyl chloride (2.60 mL, 28.4 mmol) was added. LCMS analysis showed full conversion to the desired product. The inorganic salts were filtered off and the filtrate was added to a separatory funnel containing CH₂Cl₂ and saturated aq. NaHCO₃. The aqueous phase was extracted thrice with CH₂Cl₂ and the combined organic layers were washed with brine, dried over Na₂SO₄, and concentrated. This material was purified by silica gel chromatography (150 g column, 0-10% i-PrOH/EtOAc, 30 min) and the desired fractions were combined to afford the desired compound (8.4 g, 77%) as an off-white solid. MS (ES)⁺ m/e 391.0, 392.8 [M+H]⁺. This material (80% ee) was further purified by chiral prep SFC (Chiralpak AD-H, 30×250 mm, 55% MeOH, 70 mL/min, UV 254 nm) to afford the title compound as an off-white solid (7.0 g, 99.2% ee, retention time=6.7 min.).

### d) 4-{[(3R)-1-(cyclopropylcarbonyl)-3-pyrrolidinyl]methyl}-5-[3′-(methyloxy)-4-biphenylyl]-2,4-dihydro-3H-1,2,4-triazol-3-one

To a microwave vial were added 5-(4-bromophenyl)-4-{[(3R)-1-(cyclopropylcarbonyl)-3-pyrrolidinyl]methyl}-2,4-dihydro-3H-1,2,4-triazol-3-one (30 mg, 0.077 mmol), [3-(methyloxy)phenyl]boronic acid (17.48 mg, 0.115 mmol), Cs₂CO₃ (62.5 mg, 0.192 mmol), and PdCl₂(dppf) (6.26 mg, 7.67 μmol), and the vial was purged with N₂. 1,4-Dioxane (1.0 mL) and water (0.5 mL) were added and the mixture was heated overnight to 100° C. overnight. LCMS analysis indicated desired product formation and consumption of the starting material. The mixture was filtered through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (20 mg, 63%) as a white solid. MS (ES)⁺ m/e 419.2 [M+H]⁺.

### Example 49

### N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylsulfamide

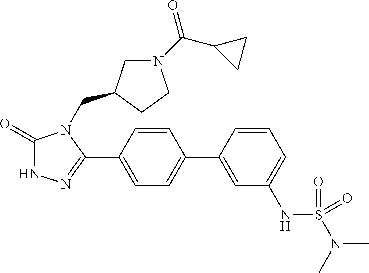 [SMILES: CN(C)S(=O)(=O)Nc1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with (3-{[(dimethylamino)sulfonyl]amino}phenyl)boronic acid to afford the title compound as an off-white solid (19 mg, 49%). MS (ES)⁺ m/e 511.0 [M+H]⁺.

### Example 50

### N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]methanesulfonamide

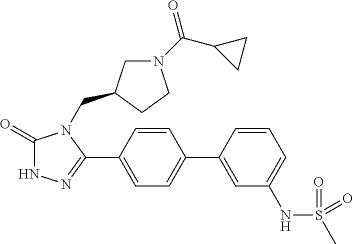 [SMILES: CS(=O)(=O)Nc1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with {3-[(methylsulfonyl)amino]phenyl}boronic acid, which provided the title compound as a white solid (23 mg, 62%). MS (ES)⁺ m/e 503.1 [M+H]⁺.

### Example 51

### N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]acetamide

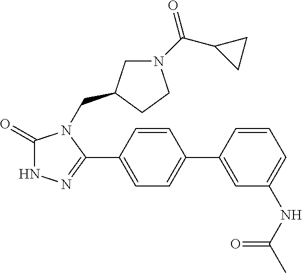 [SMILES: CC(=O)Nc1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with [3-(acetylamino)phenyl]boronic acid, which provided the title compound as a white solid (18 mg, 51%). MS (ES)⁺ m/e 446.3 [M+H]⁺.

### Example 52

### 4′-(4-{[(3R)-1-(cyclopropylcarbonyl)-3-pyrrolidinyl]methyl}-5-oxo-4,5-dihydro-1H-1,2,4-triazol-3-yl)-3-biphenylsulfonamide

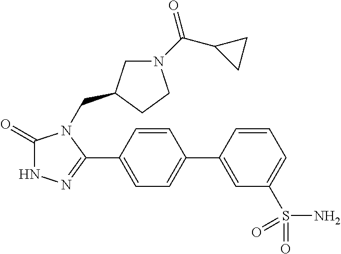 [SMILES: NS(=O)(=O)c1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with [3-(aminosulfonyl)phenyl]boronic acid, which provided the title compound as a white solid (18 mg, 50%). MS (ES)⁺ m/e 468.2 [M+H]⁺.

### Example 53

### 4′-(4-{[(3R)-1-(cyclopropylcarbonyl)-3-pyrrolidinyl]methyl}-5-oxo-4,5-dihydro-1H-1,2,4-triazol-3-yl)-3-biphenylcarboxamide

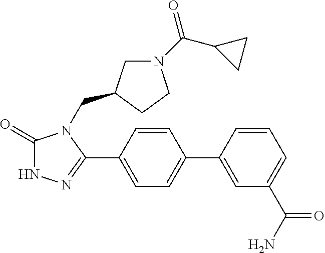 [SMILES: NC(=O)c1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with [3-(aminocarbonyl)phenyl]boronic acid, which provided the title compound as a white solid (13 mg, 39%). MS (ES)⁺ m/e 432.2 [M+H]⁺.

### Example 54

### 4′-(4-{[(3R)-1-(cyclopropylcarbonyl)-3-pyrrolidinyl]methyl}-5-oxo-4,5-dihydro-1H-1,2,4-triazol-3-yl)-3-biphenylcarbonitrile

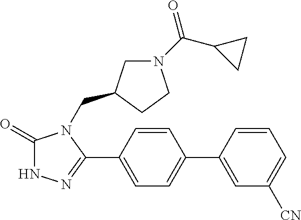 [SMILES: N#Cc1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with (3-cyanophenyl)boronic acid, which provided the title compound as a white solid (25 mg, 59%). MS (ES)⁺ m/e 414.2 [M+H]⁺.

### Example 55

### 4′-(4-{[(3R)-1-(cyclopropylcarbonyl)-3-pyrrolidinyl]methyl}-5-oxo-4,5-dihydro-1H-1,2,4-triazol-3-yl)-3-fluoro-4-biphenylcarbonitrile

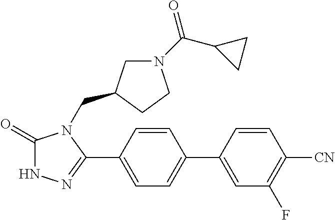 [SMILES: N#Cc1ccc(-c2ccc(-c3n[nH]c(=O)n3C[C@@H]3CCN(C(=O)C4CC4)C3)cc2)cc1F]

The procedure described for the preparation of Example 48(d) was followed, replacing [3-(methyloxy)phenyl]boronic acid with (4-cyano-3-fluorophenyl)boronic acid, provided the title compound as a white solid (24 mg, 64%). MS (ES)⁺ m/e 432.1 [M+H]⁺.

### Example 56

### 4-{[(3R)-1-(cyclopropylcarbonyl)-3-pyrrolidinyl]methyl}-5-[4-(7-methylimidazo[1,2-a]pyridin-6-yl)phenyl]-2,4-dihydro-3H-1,2,4-triazol-3-one

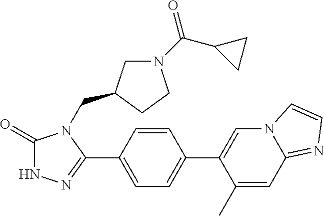 [SMILES: Cc1cc2nccn2cc1-c1ccc(-c2n[nH]c(=O)n2C[C@@H]2CCN(C(=O)C3CC3)C2)cc1]

The procedure described for the preparation of Example 48(d) was followed, replacing [3-(methyloxy)phenyl]boronic acid with (7-methylimidazo[1,2-a]pyridin-6-yl)boronic acid, which provided the title compound as a white solid (21 mg, 47%). MS (ES)⁺ m/e 443.2 [M+H]⁺.

### Example 57

### 5-(3′-acetyl-4-biphenylyl)-4-{[(3R)-1-(cyclopropylcarbonyl)-3-pyrrolidinyl]methyl}-2,4-dihydro-3H-1,2,4-triazol-3-one

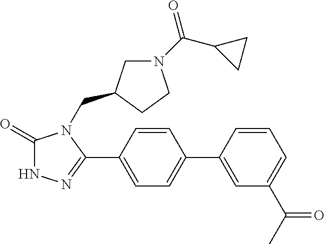 [SMILES: CC(=O)c1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with (3-acetylphenyl)boronic acid, which provided the title compound as a white solid (21 mg, 67%). MS (ES)⁺ m/e 431.1 [M+H]⁺.

### Example 58

### 4-{[(3R)-1-(cyclopropylcarbonyl)-3-pyrrolidinyl]methyl}-5-[4-(6-fluoro-3-pyridinyl)phenyl]-2,4-dihydro-3H-1,2,4-triazol-3-one

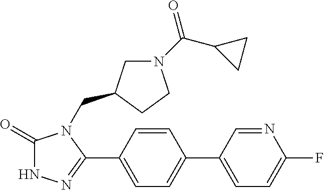 [SMILES: O=C(C1CC1)N1CC[C@@H](Cn2c(-c3ccc(-c4ccc(F)nc4)cc3)n[nH]c2=O)C1]

The procedure described for the preparation of Example 48(d) was followed, replacing [3-(methyloxy)phenyl]boronic acid with (6-fluoro-3-pyridinyl)boronic acid, provided the title compound as a white solid (26 mg, 72%). MS (ES)⁺ m/e 408.2 [M+H]⁺.

### Example 59

### 4-{[(3R)-1-(cyclopropylcarbonyl)-3-pyrrolidinyl]methyl}-5-(4-imidazo[1,2-a]pyridin-6-ylphenyl)-2,4-dihydro-3H-1,2,4-triazol-3-one

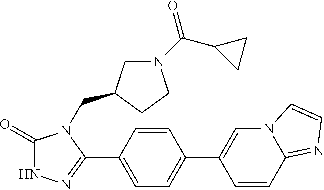 [SMILES: O=C(C1CC1)N1CC[C@@H](Cn2c(-c3ccc(-c4ccc5nccn5c4)cc3)n[nH]c2=O)C1]

The procedure described for the preparation of Example 48(d) was followed, replacing [3-(methyloxy)phenyl]boronic acid with imidazo[1,2-a]pyridin-6-ylboronic acid, provided the title compound as a white solid (22 mg, 50%). MS (ES)⁺ m/e 429.1 [M+H]⁺.

### Example 60

### 4-{[(3R)-1-(cyclopropylcarbonyl)-3-pyrrolidinyl]methyl}-5-[3′-(5-methyl-1,3,4-oxadiazol-2-yl)-4-biphenylyl]-2,4-dihydro-3H-1,2,4-triazol-3-one

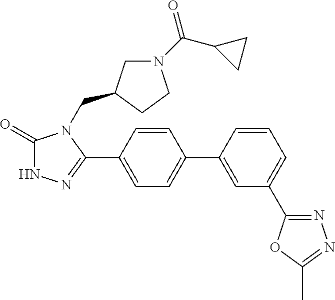 [SMILES: Cc1nnc(-c2cccc(-c3ccc(-c4n[nH]c(=O)n4C[C@@H]4CCN(C(=O)C5CC5)C4)cc3)c2)o1]

The procedure described for the preparation of Example 48(d) was followed, replacing [3-(methyloxy)phenyl]boronic acid with [3-(5-methyl-1,3,4-oxadiazol-2-yl)phenyl]boronic acid, which provided the title compound as a white solid (23 mg, 55%). MS (ES)⁺ m/e 471.2 [M+H]⁺.

### Example 61

### 5-[3′,4′-bis(methyloxy)-4-biphenylyl]-4-{[(3R)-1-(cyclopropylcarbonyl)-3-pyrrolidinyl]methyl}-2,4-dihydro-3H-1,2,4-triazol-3-one

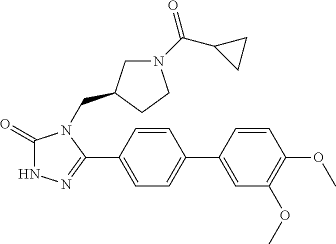 [SMILES: COc1ccc(-c2ccc(-c3n[nH]c(=O)n3C[C@@H]3CCN(C(=O)C4CC4)C3)cc2)cc1OC]

The procedure described for the preparation of Example 48(d) was followed, replacing [3-(methyloxy)phenyl]boronic acid with [3,4-bis(methyloxy)phenyl]boronic acid, which provided the title compound as a white solid (27 mg, 60%). MS (ES)⁺ m/e 449.1 [M+H]⁺.

### Example 62

### 4-{[(3R)-1-(cyclopropylcarbonyl)-3-pyrrolidinyl]methyl}-5-[4-(2,3-dihydro-1-benzofuran-5-yl)phenyl]-2,4-dihydro-3H-1,2,4-triazol-3-one

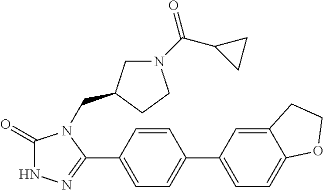 [SMILES: O=C(C1CC1)N1CC[C@@H](Cn2c(-c3ccc(-c4ccc5c(c4)CCO5)cc3)n[nH]c2=O)C1]

The procedure described for the preparation of Example 48(d) was followed, replacing [3-(methyloxy)phenyl]boronic acid with 2,3-dihydro-1-benzofuran-5-ylboronic acid, which provided the title compound as a white solid (19 mg, 50%). MS (ES)⁺ m/e 431.1 [M+H]⁺.

### Example 63

### 4-{[(3R)-1-(cyclopropylcarbonyl)-3-pyrrolidinyl]methyl}-5-{4′-[(trifluoromethyl)oxy]-4-biphenylyl}-2,4-dihydro-3H-1,2,4-triazol-3-one

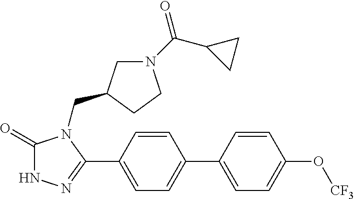 [SMILES: O=C(C1CC1)N1CC[C@@H](Cn2c(-c3ccc(-c4ccc(OC(F)(F)F)cc4)cc3)n[nH]c2=O)C1]

The procedure described for the preparation of Example 48(d) was followed, replacing [3-(methyloxy)phenyl]boronic acid with {4-[(trifluoromethyl)oxy]phenyl}boronic acid, which provided the title compound as a white solid (34 mg, 71%). MS (ES)⁺ m/e 473.3 [M+H]⁺.

### Example 64

### 4-{[(3R)-1-(cyclopropylcarbonyl)-3-pyrrolidinyl]methyl}-5-[2′,5′-difluoro-4′-(methyloxy)-4-biphenylyl]-2,4-dihydro-3H-1,2,4-triazol-3-one

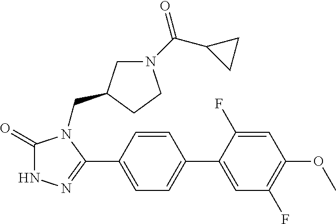 [SMILES: COc1cc(F)c(-c2ccc(-c3n[nH]c(=O)n3C[C@@H]3CCN(C(=O)C4CC4)C3)cc2)cc1F]

The procedure described for the preparation of Example 48(d) was followed, replacing [3-(methyloxy)phenyl]boronic acid with [2,5-difluoro-4-(methyloxy)phenyl]boronic acid, which provided the title compound as a white solid (27 mg, 57%). MS (ES)⁺ m/e 455.1 [M+H]⁺.

### Example 65

### 4-{[(3R)-1-(cyclopropylcarbonyl)-3-pyrrolidinyl]methyl}-5-[2′-fluoro-4′-(methyloxy)-4-biphenylyl]-2,4-dihydro-3H-1,2,4-triazol-3-one

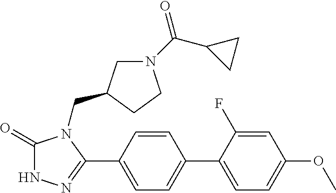 [SMILES: COc1ccc(-c2ccc(-c3n[nH]c(=O)n3C[C@@H]3CCN(C(=O)C4CC4)C3)cc2)c(F)c1]

The procedure described for the preparation of Example 48(d) was followed, replacing [3-(methyloxy)phenyl]boronic acid with [2-fluoro-4-(methyloxy)phenyl]boronic acid, which provided the title compound as a white solid (24 mg, 55%). MS (ES)⁺ m/e 437.3 [M+H]⁺.

### Example 66

4′-(4-{[(3R)-1-(cyclopropylcarbonyl)-3-pyrrolidinyl]methyl}-5-oxo-4,5-dihydro-1H-1,2,4-triazol-3-yl)-4-biphenylcarbonitrile

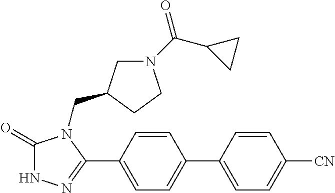 [SMILES: N#Cc1ccc(-c2ccc(-c3n[nH]c(=O)n3C[C@@H]3CCN(C(=O)C4CC4)C3)cc2)cc1]

The procedure described for the preparation of Example 48(d) was followed, replacing [3-(methyloxy)phenyl]boronic acid with (4-cyanophenyl)boronic acid, which provided the title compound as a white solid (26 mg, 62%). MS (ES)⁺ m/e 414.2 [M+H]⁺.

### Example 67

### 4′-(4-{[(3R)-1-(cyclopropylcarbonyl)-3-pyrrolidinyl]methyl}-5-oxo-4,5-dihydro-1H-1,2,4-triazol-3-yl)-4-biphenylcarboxamide

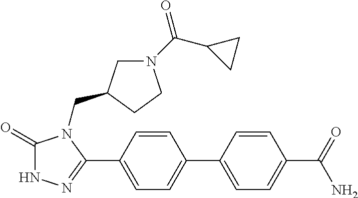 [SMILES: NC(=O)c1ccc(-c2ccc(-c3n[nH]c(=O)n3C[C@@H]3CCN(C(=O)C4CC4)C3)cc2)cc1]

The procedure described for the preparation of Example 48(d) was followed, replacing [3-(methyloxy)phenyl]boronic acid with [4-(aminocarbonyl)phenyl]boronic acid, which provided the title compound as a white solid (24 mg, 55%). MS (ES)⁺ m/e 432.2 [M+H]⁺.

### Example 68

### N-butyl-N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea

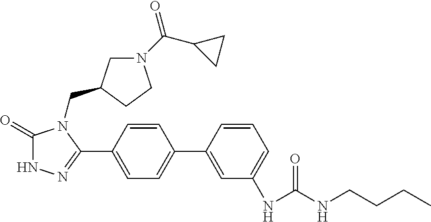 [SMILES: CCCCNC(=O)Nc1cccc(-c2ccc(-c3n[nH]c(=O)n3C[C@@H]3CCN(C(=O)C4CC4)C3)cc2)c1]

The procedure described for the preparation of Example 48(d) was followed, replacing [3-(methyloxy)phenyl]boronic acid with (3-{[(butylamino)carbonyl]amino}phenyl)boronic acid, which provided the title compound as a brown solid (20 mg, 37%). MS (ES)⁺ m/e 503.0 [M+H]⁺.

### Example 69

### 5-(2′-chloro-4′-fluoro-4-biphenylyl)-4-{[(3R)-1-(cyclopropylcarbonyl)-3-pyrrolidinyl]methyl}-2,4-dihydro-3H-1,2,4-triazol-3-one

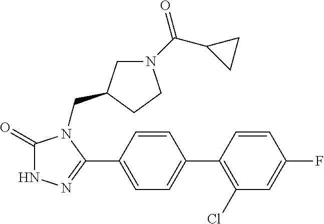 [SMILES: O=C(C1CC1)N1CC[C@@H](Cn2c(-c3ccc(-c4ccc(F)cc4Cl)cc3)n[nH]c2=O)C1]

The procedure described for the preparation of Example 48(d) was followed, replacing [3-(methyloxy)phenyl]boronic acid with (2-chloro-4-fluorophenyl)boronic acid, provided the title compound as a white solid (31 mg, 47%). MS (ES)⁺ m/e 441.2 [M+H]⁺.

### Example 70

### 4-{[(3R)-1-(cyclopropylcarbonyl)-3-pyrrolidinyl]methyl}-5-[4-(3-quinolinyl)phenyl]-2,4-dihydro-3H-1,2,4-triazol-3-one

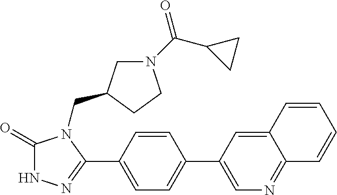 [SMILES: O=C(C1CC1)N1CC[C@@H](Cn2c(-c3ccc(-c4cnc5ccccc5c4)cc3)n[nH]c2=O)C1]

The procedure described for the preparation of Example 48(d) was followed, replacing [3-(methyloxy)phenyl]boronic acid with 3-(4,4,5,5-tetramethyl-1,3,2-dioxaborolan-2-yl)quinoline, which provided the title compound as a white solid (28 mg, 50%). MS (ES)⁺ m/e 440.1 [M+H]⁺.

### Example 71

### 4-{[(3R)-1-(cyclopropylcarbonyl)-3-pyrrolidinyl]methyl}-5-[4-(5-quinolinyl)phenyl]-2,4-dihydro-3H-1,2,4-triazol-3-one

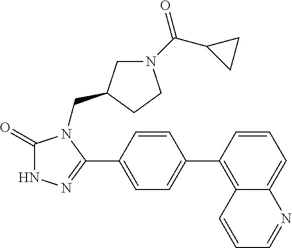 [SMILES: O=C(C1CC1)N1CC[C@@H](Cn2c(-c3ccc(-c4cccc5ncccc45)cc3)n[nH]c2=O)C1]

The procedure described for the preparation of Example 48(d) was followed, replacing [3-(methyloxy)phenyl]boronic acid with 5-(4,4,5,5-tetramethyl-1,3,2-dioxaborolan-2-yl)quinoline, which provided the title compound as a white solid (39 mg, 70%). MS (ES)⁺ m/e 440.1 [M+H]⁺.

### Example 72

### 4-{[(3R)-1-(cyclopropylcarbonyl)-3-pyrrolidinyl]methyl}-5-[4-(6-quinolinyl)phenyl]-2,4-dihydro-3H-1,2,4-triazol-3-one

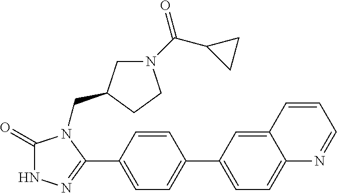 [SMILES: O=C(C1CC1)N1CC[C@@H](Cn2c(-c3ccc(-c4ccc5ncccc5c4)cc3)n[nH]c2=O)C1]

The procedure described for the preparation of Example 48(d) was followed, replacing [3-(methyloxy)phenyl]boronic acid with 6-(4,4,5,5-tetramethyl-1,3,2-dioxaborolan-2-yl)quinoline, which provided the title compound as a white solid (29 mg, 49%). MS (ES)⁺ m/e 440.1 [M+H]⁺.

### Example 73

### 4-{[(3R)-1-(cyclopropylcarbonyl)-3-pyrrolidinyl]methyl}-5-[4-(7-quinolinyl)phenyl]-2,4-dihydro-3H-1,2,4-triazol-3-one

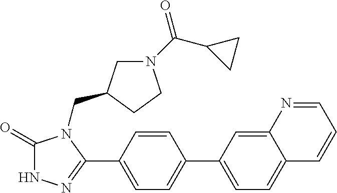 [SMILES: O=C(C1CC1)N1CC[C@@H](Cn2c(-c3ccc(-c4ccc5cccnc5c4)cc3)n[nH]c2=O)C1]

The procedure described for the preparation of Example 48(d) was followed, replacing [3-(methyloxy)phenyl]boronic acid with 7-(4,4,5,5-tetramethyl-1,3,2-dioxaborolan-2-yl)quinoline, which provided the title compound as a white solid (29 mg, 48%). MS (ES)⁺ m/e 440.1 [M+H]⁺.

### Example 74

### 4-{[(3R)-1-(cyclopropylcarbonyl)-3-pyrrolidinyl]methyl}-5-[3′-hydroxy-4′-(methyloxy)-4-biphenylyl]-2,4-dihydro-3H-1,2,4-triazol-3-one

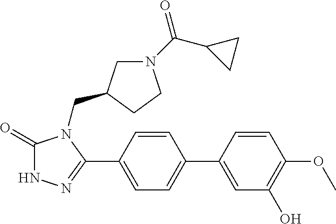 [SMILES: COc1ccc(-c2ccc(-c3n[nH]c(=O)n3C[C@@H]3CCN(C(=O)C4CC4)C3)cc2)cc1O]

The procedure described for the preparation of Example 48(d) was followed, replacing [3-(methyloxy)phenyl]boronic acid with 2-(methyloxy)-5-(4,4,5,5-tetramethyl-1,3,2-dioxaborolan-2-yl)phenol, which provided the title compound as a white solid (28 mg, 52%). MS (ES)⁺ m/e 435.3 [M+H]⁺.

### Example 75

### 4-{[(3R)-1-(cyclopropylcarbonyl)-3-pyrrolidinyl]methyl}-5-[3′-methyl-4′-(methyloxy)-4-biphenylyl]-2,4-dihydro-3H-1,2,4-triazol-3-one

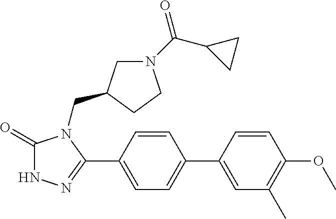 [SMILES: COc1ccc(-c2ccc(-c3n[nH]c(=O)n3C[C@@H]3CCN(C(=O)C4CC4)C3)cc2)cc1C]

The procedure described for the preparation of Example 48(d) was followed, replacing [3-(methyloxy)phenyl]boronic acid with 4,4,5,5-tetramethyl-2-[3-methyl-4-(methyloxy)phenyl]-1,3,2-dioxaborolane, which provided the title compound as a white solid (42 mg, 75%). MS (ES)⁺ m/e 433.3 [M+H]⁺.

### Example 76

### 4-{[(3R)-1-(cyclopropylcarbonyl)-3-pyrrolidinyl]methyl}-5-[4-(8-methyl-5-quinolinyl)phenyl]-2,4-dihydro-3H-1,2,4-triazol-3-one

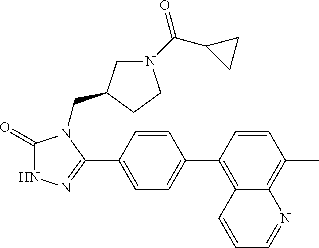 [SMILES: Cc1ccc(-c2ccc(-c3n[nH]c(=O)n3C[C@@H]3CCN(C(=O)C4CC4)C3)cc2)c2cccnc12]

The procedure described for the preparation of Example 48(d) was followed, replacing [3-(methyloxy)phenyl]boronic acid with 8-methyl-5-(4,4,5,5-tetramethyl-1,3,2-dioxaborolan-2-yl)quinoline, which provided the title compound as a white solid (38 mg, 64%). MS (ES)⁺ m/e 454.1 [M+H]⁺.

### Example 77

### 5-(4′-chloro-2′,6′-difluoro-4-biphenylyl)-4-{[(3R)-1-(cyclopropylcarbonyl)-3-pyrrolidinyl]methyl}-2,4-dihydro-3H-1,2,4-triazol-3-one

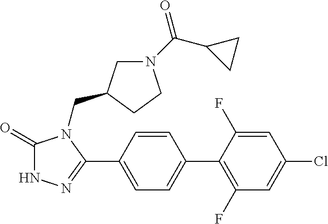 [SMILES: O=C(C1CC1)N1CC[C@@H](Cn2c(-c3ccc(-c4c(F)cc(Cl)cc4F)cc3)n[nH]c2=O)C1]

To a microwave vial were added 5-(4-bromophenyl)-4-{[(3R)-1-(cyclopropylcarbonyl)-3-pyrrolidinyl]methyl}-2,4-dihydro-3H-1,2,4-triazol-3-one (61 mg, 0.156 mmol), (4-chloro-2,6-difluorophenyl)boronic acid (46 mg, 0.239 mmol), Pd(P-t-Bu₃)₂ (4 mg, 7.83 μmol), CsF (47 mg, 0.309 mmol), and silver oxide (48 mg, 0.207 mmol), and the vial was purged with N₂. Dry DMF (1.1 mL) was added and the mixture was heated overnight on a hot plate at 100° C. LCMS analysis indicated desired product formation and consumption of the starting material. The mixture was filtered through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (15 mg, 21%) as a white solid. MS (ES)⁺ m/e 459.2 [M+H]⁺.

### Example 78

### 4-{[(3R)-1-(cyclopropylcarbonyl)-3-pyrrolidinyl]methyl}-5-(4-imidazo[1,2-a]pyridin-2-ylphenyl)-2,4-dihydro-3H-1,2,4-triazol-3-one

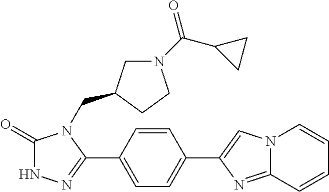 [SMILES: O=C(C1CC1)N1CC[C@@H](Cn2c(-c3ccc(-c4cn5ccccc5n4)cc3)n[nH]c2=O)C1]

To a microwave vial were added 5-(4-bromophenyl)-4-{[(3R)-1-(cyclopropylcarbonyl)-3-pyrrolidinyl]methyl}-2,4-dihydro-3H-1,2,4-triazol-3-one (60 mg, 0.153 mmol), 4,4,4′,4′,5,5,5′,5′-octamethyl-2,2′-bi-1,3,2-dioxaborolane (46.7 mg, 0.184 mmol), KOAc (52.7 mg, 0.537 mmol), and PdCl₂(dppf) (25.05 mg, 0.031 mmol), and the vial was purged with N₂. 1,4-Dioxane (1.0 mL) was added and the mixture was heated to 110° C. for 5 h. LCMS analysis indicated consumption of the starting material and formation of the boronic ester intermediate. 2-Bromoimidazo[1,2-a]pyridine (22.16 mg, 0.112 mmol), Cs₂CO₃ (100 mg, 0.307 mmol), PdCl₂(dppf) (25.05 mg, 0.031 mmol), and water (0.5 mL) were added to the reaction mixture. The reaction mixture was irradiated in a microwave reactor at 110° C. for 30 min. LCMS analysis indicated desired product formation. The mixture was filtered through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (6 mg, 13%) as a white solid. MS (ES)⁺ m/e 429.1 [M+H]⁺.

### Example 79

### 4-{[(3R)-1-(cyclopropylcarbonyl)-3-pyrrolidinyl]methyl}-5-(4-imidazo[1,2-a]pyridin-7-ylphenyl)-2,4-dihydro-3H-1,2,4-triazol-3-one

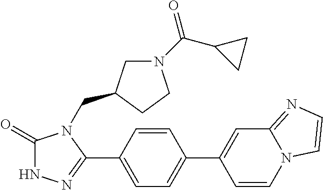 [SMILES: O=C(C1CC1)N1CC[C@@H](Cn2c(-c3ccc(-c4ccn5ccnc5c4)cc3)n[nH]c2=O)C1]

The procedure described for the preparation of Example 78 was followed, replacing 2-bromoimidazo[1,2-a]pyridine with 7-bromoimidazo[1,2-a]pyridine, which provided the title compound as a white solid (19 mg, 34%). MS (ES)⁺ m/e 429.1 [M+H]⁺.

### Example 80

### 4-{[(3R)-1-(cyclopropylcarbonyl)-3-pyrrolidinyl]methyl}-5-[4-(8-isoquinolinyl)phenyl]-2,4-dihydro-3H-1,2,4-triazol-3-one

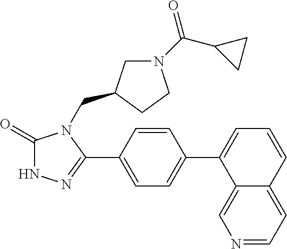 [SMILES: O=C(C1CC1)N1CC[C@@H](Cn2c(-c3ccc(-c4cccc5ccncc45)cc3)n[nH]c2=O)C1]

The procedure described for the preparation of Example 78 was followed replacing, 2-bromoimidazo[1,2-a]pyridine with 8-bromoisoquinoline, which provided the title compound as a tan solid (4 mg, 9%). MS (ES)⁺ m/e 440.1 [M+H]⁺.

### Example 81

### 5-[4-(3-amino-1H-indazol-6-yl)phenyl]-4-{[(3R)-1-(cyclopropylcarbonyl)-3-pyrrolidinyl]methyl}-2,4-dihydro-3H-1,2,4-triazol-3-one

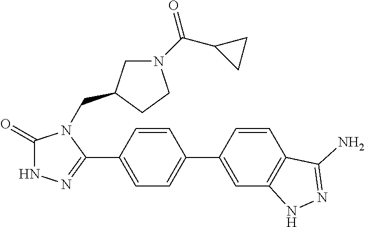 [SMILES: Nc1n[nH]c2cc(-c3ccc(-c4n[nH]c(=O)n4C[C@@H]4CCN(C(=O)C5CC5)C4)cc3)ccc12]

To a microwave vial was added 4′-(4-{[(3R)-1-(cyclopropylcarbonyl)-3-pyrrolidinyl]methyl}-5-oxo-4,5-dihydro-1H-1,2,4-triazol-3-yl)-3-fluoro-4-biphenylcarbonitrile (100 mg, 0.232 mmol), and the vial was purged with N₂. EtOH (1.0 mL) and hydrazine (72.7 μl, 2.318 mmol) were added and the mixture was irradiated in a microwave reactor for 20 min. at 110° C. LCMS analysis indicated desired product formation and consumption of the starting material. The mixture was filtered through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (25 mg, 24%) as a white solid. MS (ES)⁺ m/e 444.3 [M+H]⁺.

### Example 82

### 5-[4-(1,3-benzoxazol-5-yl)phenyl]-4-{[(3R)-1-(cyclopropylcarbonyl)-3-pyrrolidinyl]methyl}-2,4-dihydro-3H-1,2,4-triazol-3-one

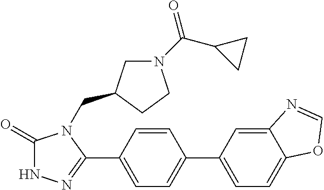 [SMILES: O=C(C1CC1)N1CC[C@@H](Cn2c(-c3ccc(-c4ccc5ocnc5c4)cc3)n[nH]c2=O)C1]

To a microwave vial were added 5-bromo-1,3-benzoxazole (50 mg, 0.253 mmol), 4,4,4′,4′,5,5,5′,5′-octamethyl-2,2′-bi-1,3,2-dioxaborolane (77 mg, 0.303 mmol), KOAc (74.3 mg, 0.758 mmol), and PdCl₂(dppf) (41 mg, 0.051 mmol), and the vial was purged with N₂. 1,4-Dioxane (1.0 mL) was added and the mixture was heated at 110° C. for 6 h. LCMS analysis indicated consumption of the starting material and formation of the boronic ester intermediate. 5-(4-Bromophenyl)-4-{[(3R)-1-(cyclopropylcarbonyl)-3-pyrrolidinyl]methyl}-2,4-dihydro-3H-1,2,4-triazol-3-one (79 mg, 0.202 mmol), Cs₂CO₃ (165 mg, 0.505 mmol), PdCl₂(dppf) (41 mg, 0.051 mmol), and water (0.5 mL) were added and the resulting mixture was irradiated in a microwave reactor at 110° C. for 30 min. LCMS analysis showed desired product formation. The mixture was filtered through a syringe filter and purified by reverse phase HPLC (Xbridge C-18: 19×100 mm column; 10-90% CH₃CN/water+0.1% NH₄OH) The desired fractions were collected and added to a reparatory funnel containing EtOAc and saturated aq. NaHCO₃. The aqueous phase was extracted thrice with EtOAc and the combined organic layers were washed with brine, dried over Na₂SO₄, and concentrated to afford the title compound (11 mg, 10%) as a white solid. MS (ES)⁺ m/e 430.2 [M+H]⁺.

### Example 83

### N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3-biphenylyl]-N,N-dimethylsulfamide

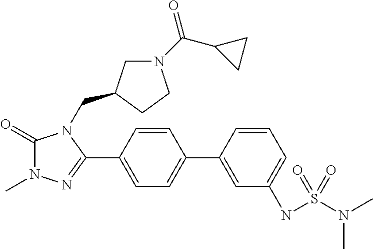 [SMILES: CN(C)S(=O)(=O)Nc1cccc(-c2ccc(-c3nn(C)c(=O)n3C[C@@H]3CCN(C(=O)C4CC4)C3)cc2)c1]

To a microwave vial were added 5-(4-bromophenyl)-4-{[(3R)-1-(cyclopropylcarbonyl)-3-pyrrolidinyl]methyl}-2-methyl-2,4-dihydro-3H-1,2,4-triazol-3-one (55 mg, 0.136 mmol), (3-{[(dimethylamino)sulfonyl]amino}phenyl)boronic acid (55 mg, 0.225 mmol), Cs₂CO₃ (111 mg, 0.339 mmol), and PdCl₂(dppf) (11.08 mg, 0.014 mmol), and the vial was purged with N₂. 1,4-Dioxane (1 mL) and water (0.5 mL) were added and the mixture was heated overnight on a hot plate at 100° C. LCMS analysis indicated desired product formation and consumption of the starting material. The mixture was filtered through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (52 mg, 73%) as an off-white solid. MS (ES)⁺ m/e 525.3 [M+H]⁺.

### Example 84

### 4′-(4-{[(3R)-1-(cyclopropylcarbonyl)-3-pyrrolidinyl]methyl}-1-methyl-5-oxo-4,5-dihydro-1H-1,2,4-triazol-3-yl)-3-fluoro-4-biphenylcarbonitrile

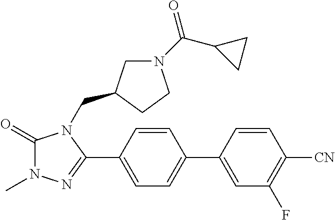 [SMILES: Cn1nc(-c2ccc(-c3ccc(C#N)c(F)c3)cc2)n(C[C@@H]2CCN(C(=O)C3CC3)C2)c1=O]

The procedure described for the preparation of Example 83 was followed, replacing (3-{[(dimethylamino)sulfonyl]amino}phenyl)boronic acid with (4-cyano-3-fluorophenyl)boronic acid, which provided the title compound as a tan solid (11 mg, 17%). MS (ES)⁺ m/e 446.3 [M+H]⁺.

### Example 85

### 4-{[(3R)-1-(cyclopropylcarbonyl)-3-pyrrolidinyl]methyl}-5-(4-imidazo[1,2-a]pyridin-6-ylphenyl)-2-methyl-2,4-dihydro-3H-1,2,4-triazol-3-one

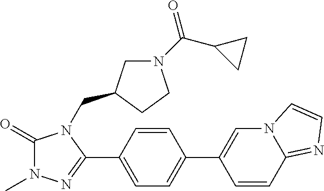 [SMILES: Cn1nc(-c2ccc(-c3ccc4nccn4c3)cc2)n(C[C@@H]2CCN(C(=O)C3CC3)C2)c1=O]

The procedure described for the preparation of Example 83 was followed, replacing (3-{[(dimethylamino)sulfonyl]amino}phenyl)boronic acid with imidazo[1,2-a]pyridin-6-ylboronic acid, which provided the title compound as a tan solid (15 mg, 27%). MS (ES)⁺ m/e 444.2 [M+H]⁺.

### Example 86

### 5-[4-(3-amino-1H-indazol-6-yl)phenyl]-4-{[(3R)-1-(cyclopropylcarbonyl)-3-pyrrolidinyl]methyl}-2-methyl-2,4-dihydro-3H-1,2,4-triazol-3-one

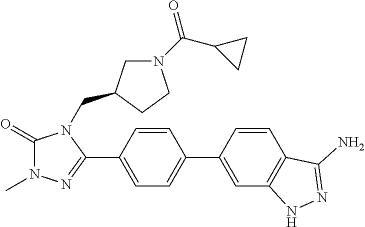 [SMILES: Cn1nc(-c2ccc(-c3ccc4c(N)n[nH]c4c3)cc2)n(C[C@@H]2CCN(C(=O)C3CC3)C2)c1=O]

The procedure described for the preparation of Example 81 was followed replacing 4′-(4-{[(3R)-1-(cyclopropylcarbonyl)-3-pyrrolidinyl]methyl}-5-oxo-4,5-dihydro-1H-1,2,4-triazol-3-yl)-3-fluoro-4-biphenylcarbonitrile with 4′-(4-{[(3R)-1-(cyclopropylcarbonyl)-3-pyrrolidinyl]methyl}-1-methyl-5-oxo-4,5-dihydro-1H-1,2,4-triazol-3-yl)-3-fluoro-4-biphenylcarbonitrile, which provided the title compound as a white solid (25 mg, 56%). MS (ES)⁺ m/e 458.3 [M+H]⁺.

### Example 87

### 5-[4-(4-{[(3R)-1-(cyclopropylcarbonyl)-3-pyrrolidinyl]methyl}-5-oxo-4,5-dihydro-1H-1,2,4-triazol-3-yl)phenyl]-3-methyl-1-benzofuran-2-carboxylic acid

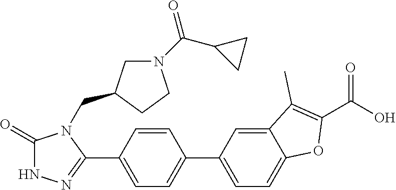 [SMILES: Cc1c(C(=O)O)oc2ccc(-c3ccc(-c4n[nH]c(=O)n4C[C@@H]4CCN(C(=O)C5CC5)C4)cc3)cc12]

### a) ethyl 5-bromo-3-methyl-1-benzofuran-2-carboxylate

To a microwave vial, 1-(5-bromo-2-hydroxyphenyl)ethanone (0.992 g, 4.61 mmol) and Cs₂CO₃ (4.85 g, 14.89 mmol) were added and purged with N₂. CH₃CN (15.38 mL) and ethyl bromoacetate (0.775 mL, 6.99 mmol) were added to the mixture. The suspension was heated at 100° C. for 65 h, and LCMS analysis indicated conversion to the desired product. The solution was acidified to pH=3 with 1 N aq. HCl and EtOAc (50 mL) was added. The aqueous phase was extracted thrice with EtOAc and the combined organic layers were washed with brine, dried over Na₂SO₄, and concentrated to dryness. This residue was triturated with a CH₂Cl₂:Et₂O:heptane (˜1:1:1) to filter off an undesired brown solid. The filtrate was concentrated and purified by silica gel chromatography (24 g column; 1-25% EtOAc/hexanes), and the desired fractions were combined and concentrated to afford the title compound (681 mg, 52%) as a white solid. MS (ES)⁺ m/e 283.0, 285.0 [M+H]⁺.

### b) 5-[4-(4-{[(3R)-1-(cyclopropylcarbonyl)-3-pyrrolidinyl]methyl}-5-oxo-4,5-dihydro-1H-1,2,4-triazol-3-yl)phenyl]-3-methyl-1-benzofuran-2-carboxylic acid

The procedure described for the preparation of Example 78 was followed replacing 2-bromoimidazo[1,2-a]pyridine with ethyl 5-bromo-3-methyl-1-benzofuran-2-carboxylate, which provided the title compound as a white solid (7 mg, 17%). MS (ES)⁺ m/e 487.3 [M+H]⁺.

### Example 88

### 4-{[(3R)-1-(cyclopropylcarbonyl)-3-pyrrolidinyl]methyl}-5-[4-(3-methyl-1-benzofuran-5-yl)phenyl]-2,4-dihydro-3H-1,2,4-triazol-3-one

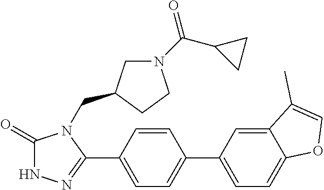 [SMILES: Cc1coc2ccc(-c3ccc(-c4n[nH]c(=O)n4C[C@@H]4CCN(C(=O)C5CC5)C4)cc3)cc12]

### a) 5-bromo-3-methyl-1-benzofuran-2-carboxylic acid

To a reaction vial were added ethyl 5-bromo-3-methyl-1-benzofuran-2-carboxylate (700 mg, 2.472 mmol) and KOH (600 mg, 10.69 mmol). MeOH (5 mL) and THF (5.00 mL) were added and the mixture was allowed to stir at room temperature for 2 h. LCMS analysis indicated conversion to the desired product. The solution was acidified to pH=3 with 1 N aq. HCl and EtOAc (40 mL) was added. The aqueous phase was extracted thrice with EtOAc and the combined organic layers were washed with brine, dried over Na₂SO₄, and concentrated to dryness to afford the title compound (500 mg, 81%) as an off-white solid. MS (ES)⁺ m/e 254.9, 256.9 [M+H]⁺.

### b) 5-bromo-3-methyl-1-benzofuran

To a microwave vial were added [(2-acetyl-4-bromophenyl)oxy]acetic acid (450 mg, 1.648 mmol) and NaOAc (811 mg, 9.89 mmol), and the vial was purged with N₂. Acetic anhydride (2021 μl, 21.42 mmol) and HOAc (2075 μl, 36.3 mmol) were added and the mixture was heated at 140° C. for 3.5 h. LCMS analysis shows one major product and consumption of the starting material. The mixture was diluted with water and EtOAc and the layers were separated. The organic layer was washed with 1 N aq. NaOH until the aqueous phase was basic (pH=12). The aqueous phase was extracted thrice with EtOAc and the combined organic layers were washed with brine, dried over Na₂SO₄, and concentrated to dryness to afford the title compound (310 mg, 89%) as an oil. ¹H NMR (400 MHz, CDCl₃) δ ppm 7.67 (d, J=1.77 Hz, 1H), 7.38-7.45 (m, 2H), 7.32-7.38 (m, 1H), 2.24 (d, J=1.26 Hz, 3H).

### c) 4-{[(3R)-1-(cyclopropylcarbonyl)-3-pyrrolidinyl]methyl}-5-[4-(3-methyl-1-benzofuran-5-yl)phenyl]-2,4-dihydro-3H-1,2,4-triazol-3-one

The procedure described for the preparation of Example 78 was followed replacing 2-bromoimidazo[1,2-a]pyridine with 5-bromo-3-methyl-1-benzofuran, which provided the title compound as a tan solid (20 mg, 29%). MS (ES)⁺ m/e 443.2 [M+H]⁺.

### Example 89

### 5-[4-(4-{[(3R)-1-(cyclopropylcarbonyl)-3-pyrrolidinyl]methyl}-5-oxo-4,5-dihydro-1H-1,2,4-triazol-3-yl)phenyl]-1H-indole-3-carbonitrile

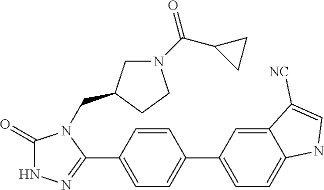 [SMILES: N#Cc1c[nH]c2ccc(-c3ccc(-c4n[nH]c(=O)n4C[C@@H]4CCN(C(=O)C5CC5)C4)cc3)cc12]

### a) 5-bromo-1H-indole-3-carbonitrile

To a round bottom flask, 5-bromoindole (1.032 g, 5.26 mmol) was added and the flask was purged with N₂. Dry CH₃CN (10 mL) was added under N₂ and the resulting solution was cooled to −45° C. In a separate vial, chlorosulfonyl isocyanate (0.487 mL, 5.61 mmol) was dissolved in dry CH₃CN (3 mL) under N₂ and cooled to −45° C. This solution was added dropwise to the reaction mixture. This mixture was stirred at −45° C. for 15 min. LCMS analysis showed consumption of the starting material. Dry DMF (10.19 mL, 132 mmol) was added slowly and the mixture was allowed to warm to room temperature. After 1 h, LCMS analysis shows complete conversion to the desired product. The solution was added to a separatory funnel containing EtOAc and saturated aq. NaHCO₃. The aqueous phase was extracted four times with EtOAc and the combined organic layers were washed twice with brine, dried over Na₂SO₄, and concentrated to afford the title compound (1.05 g, 90%) as a white solid. MS (ES)⁺ m/e 220.9, 222.8 [M+H]⁺.

### b) 5-[4-(4-{[(3R)-1-(cyclopropylcarbonyl)-3-pyrrolidinyl]methyl}-5-oxo-4,5-dihydro-1H-1,2,4-triazol-3-yl)phenyl]-1H-indole-3-carbonitrile

The procedure described for the preparation of Example 78 was followed replacing 2-bromoimidazo[1,2-a]pyridine with 5-bromo-1H-indole-3-carbonitrile, which provided the title compound as a white solid (20 mg, 28%). MS (ES)⁺ m/e 453.2 [M+H]⁺.

### Example 90

### 5-[4-(1-benzofuran-5-yl)phenyl]-4-{[(3R)-1-(cyclopentylcarbonyl)-3-pyrrolidinyl]methyl}-2,4-dihydro-3H-1,2,4-triazol-3-one

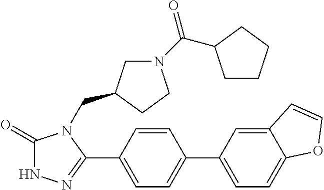 [SMILES: O=C(C1CCCC1)N1CC[C@@H](Cn2c(-c3ccc(-c4ccc5occc5c4)cc3)n[nH]c2=O)C1]

### a) 1,1-dimethylethyl(3S)-3-{[3-(4-bromophenyl)-5-oxo-1,5-dihydro-4H-1,2,4-triazol-4-yl]methyl}-1-pyrrolidinecarboxylate

To a round bottom flask were added 5-(4-bromophenyl)-4-[(3R)-3-pyrrolidinylmethyl]-2,4-dihydro-3H-1,2,4-triazol-3-one (12 g, 37 mmol) and NMP (75 mL), and the mixture was cooled to −78° C. Boc₂O (9.48 mL, 40.8 mmol) and DIPEA (25.9 mL, 149 mmol) were added and the mixture was allowed to warm to room temperature. After 3 h, LCMS analysis indicated desired product formation. The mixture was added to a separatory funnel containing EtOAc and saturated aq. NaHCO₃. The aqueous phase was extracted four times with EtOAc and the combined organic layers were washed twice with brine, dried over Na₂SO₄, and concentrated. The residue was purified using silica gel chromatography (200 g column; 10-100% EtOAc/hexanes). The desired fractions were collected and concentrated to a white solid. This material (80% ee) was further purified by chiral prep SFC (Chiralpak AD-H, 30×250 mm, 35% MeOH, 70 mL/min, UV 254 nm) to afford the title compound (3.4 g, 99. % ee) as a white solid. MS (ES)⁺ m/e 423.0, 424.8 [M+H]⁺.

### b) 1,1-dimethylethyl(3S)-3-({3-[4-(1-benzofuran-5-yl)phenyl]-5-oxo-1,5-dihydro-4H-1,2,4-triazol-4-yl}methyl)-1-pyrrolidinecarboxylate

To a microwave vial were added 1,1-dimethylethyl(3S)-3-{[3-(4-bromophenyl)-5-oxo-1,5-dihydro-4H-1,2,4-triazol-4-yl]methyl}-1-pyrrolidinecarboxylate (400 mg, 0.945 mmol), 5-(4,4,5,5-tetramethyl-1,3,2-dioxaborolan-2-yl)-1-benzofuran (390 mg, 1.598 mmol), Cs₂CO₃ (1089 mg, 3.34 mmol), and Pd(P-t-Bu₃)₂ (38 mg, 0.074 mmol), and the mixture was purged with N₂. 1,4-Dioxane (8 mL) and water (3 mL) were added and the reaction mixture was heated at 90° C. for 16 h. LCMS analysis indicated desired product formation and consumption of the starting material. The mixture was filtered and washed with EtOAc. The filtrate was added to a separatory funnel containing saturated aq. NaHCO₃. The aqueous phase was extracted thrice with EtOAc and the combined organic layers were washed with brine, dried over Na₂SO₄, and concentrated. The residue was purified by silica gel chromatography (40 g column, 5-100% EtOAc/hexanes). The desired fractions were collected and concentrated to afford the title compound (350 mg, 80%) as an off-white solid. MS (ES)⁺ m/e 461.3, 463.5 [M+H]⁺.

### c) 5-[4-(1-benzofuran-5-yl)phenyl]-4-[(3R)-3-pyrrolidinylmethyl]-2,4-dihydro-3H-1,2,4-triazol-3-one hydrochloride

To a round bottom flask were added 1,1-dimethylethyl(3S)-3-({3-[4-(1-benzofuran-5-yl)phenyl]-5-oxo-1,5-dihydro-4H-1,2,4-triazol-4-yl}methyl)-1-pyrrolidinecarboxylate (325 mg, 0.706 mmol) and HCl (3 mL, 4 M in 1,4-dioxane, 12.0 mmol) and the mixture was stirred at room temperature for 1 h. LCMS analysis indicated complete conversion to the desired product. The solution was concentrated to afford the title compound (275 mg, 98%) as a white solid. MS (ES)⁺ m/e 361.1 [M+H]⁺.

### d) 5-[4-(1-benzofuran-5-yl)phenyl]-4-{[(3R)-1-(cyclopentylcarbonyl)-3-pyrrolidinyl]methyl}-2,4-dihydro-3H-1,2,4-triazol-3-one

To a reaction vial was added 5-[4-(1-benzofuran-5-yl)phenyl]-4-[(3R)-3-pyrrolidinylmethyl]-2,4-dihydro-3H-1,2,4-triazol-3-one hydrochloride (52 mg, 0.131 mmol), and the vial was purged with N₂. NMP (0.8 mL) and DIPEA (105 μl, 0.600 mmol) were added and the mixture was cooled to −78° C. Cyclopentanecarbonyl chloride (16.04 μl, 0.132 mmol) in NMP (0.2 mL) was added and the reaction mixture was allowed to warm to room temperature and stirred for 1 h. LCMS analysis indicated desired product formation. The mixture was purified by reverse phase HPLC (10-90% CH₃CN/water+0.1% TFA). The desired fractions were collected and added to a reparatory funnel containing EtOAc and saturated aq. NaHCO₃. The aqueous phase was extracted thrice with EtOAc and the combined organic layers were washed with brine, dried over Na₂SO₄, and concentrated to afford the title compound (45 mg, 82%) as an off-white solid. MS (ES)⁺ m/e 457.2 [M+H]⁺.

### Example 91

### 5-[4-(1-benzofuran-5-yl)phenyl]-4-{[(3R)-1-(2-methylpropanoyl)-3-pyrrolidinyl]methyl}-2,4-dihydro-3H-1,2,4-triazol-3-one

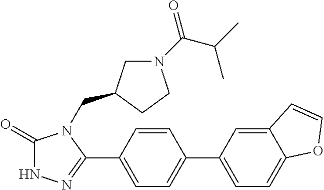 [SMILES: CC(C)C(=O)N1CC[C@@H](Cn2c(-c3ccc(-c4ccc5occc5c4)cc3)n[nH]c2=O)C1]

The procedure described for the preparation of Example 90(d) was followed replacing cyclopentanecarbonyl chloride with 2-methylpropanoyl chloride, which provided the title compound as a white solid (28 mg, 69%). MS (ES)⁺ m/e 431.1 [M+H]⁺.

### Example 92

### (3R)-3-({3-[4-(1-benzofuran-5-yl)phenyl]-5-oxo-1,5-dihydro-4H-1,2,4-triazol-4-yl}methyl)-N,N-dimethyl-1-pyrrolidinecarboxamide

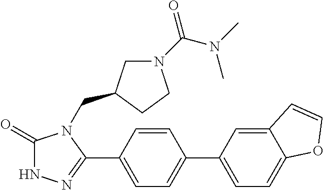 [SMILES: CN(C)C(=O)N1CC[C@@H](Cn2c(-c3ccc(-c4ccc5occc5c4)cc3)n[nH]c2=O)C1]

The procedure described for the preparation of Example 90(d) was followed replacing cyclopentanecarbonyl chloride with dimethylcarbamic chloride, which provided the title compound as a white solid (15 mg, 38%). MS (ES)⁺ m/e 432.0 [M+H]⁺.

### Example 93

### 5-[4-(1-benzofuran-5-yl)phenyl]-4-{[(3R)-1-propanoyl-3-pyrrolidinyl]methyl}-2,4-dihydro-3H-1,2,4-triazol-3-one

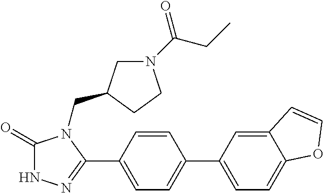 [SMILES: CCC(=O)N1CC[C@@H](Cn2c(-c3ccc(-c4ccc5occc5c4)cc3)n[nH]c2=O)C1]

The procedure described for the preparation of Example 90(d) was followed replacing cyclopentanecarbonyl chloride with propanoyl chloride, which provided the title compound as a white solid (25 mg, 66%). MS (ES)⁺ m/e 417.2 [M+H]⁺.

### Example 94

### Methyl(3S)-3-({3-[4-(1-benzofuran-5-yl)phenyl]-5-oxo-1,5-dihydro-4H-1,2,4-triazol-4-yl}methyl)-1-pyrrolidinecarboxylate

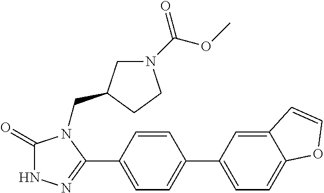 [SMILES: COC(=O)N1CC[C@@H](Cn2c(-c3ccc(-c4ccc5occc5c4)cc3)n[nH]c2=O)C1]

The procedure described for the preparation of Example 90(d) was followed replacing cyclopentanecarbonyl chloride with methyl chloroformate, which provided the title compound as a yellow solid (20 mg, 69%). MS (ES)⁺ m/e 419.2 [M+H]⁺.

### Example 95

### 4-{[(3R)-1-acetyl-3-pyrrolidinyl]methyl}-5-[4-(1-benzofuran-5-yl)phenyl]-2,4-dihydro-3H-1,2,4-triazol-3-one

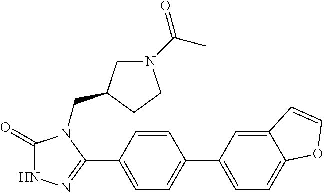 [SMILES: CC(=O)N1CC[C@@H](Cn2c(-c3ccc(-c4ccc5occc5c4)cc3)n[nH]c2=O)C1]

The procedure described for the preparation of Example 90(d) was followed replacing cyclopentanecarbonyl chloride with acetyl chloride, which provided the title compound as a yellow solid (30 mg, 74%). MS (ES)⁺ m/e 403.0 [M+H]⁺.

### Example 96

### 5-[4-(1-benzofuran-5-yl)phenyl]-4-{[(3R)-1-(2,2-dimethylpropanoyl)-3-pyrrolidinyl]methyl}-2,4-dihydro-3H-1,2,4-triazol-3-one

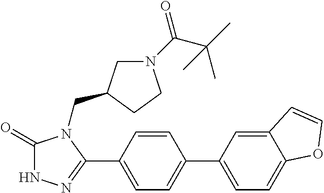 [SMILES: CC(C)(C)C(=O)N1CC[C@@H](Cn2c(-c3ccc(-c4ccc5occc5c4)cc3)n[nH]c2=O)C1]

The procedure described for the preparation of Example 90(d) was followed replacing cyclopentanecarbonyl chloride with pivaloyl chloride, which provided the title compound as a yellow solid (25 mg, 64%). MS (ES)⁺ m/e 445.2 [M+H]⁺.

### Example 97

### 5-[4-(1-benzofuran-5-yl)phenyl]-4-({(3R)-1-[(1-methylcyclopropyl)carbonyl]-3-pyrrolidinyl}methyl)-2,4-dihydro-3H-1,2,4-triazol-3-one

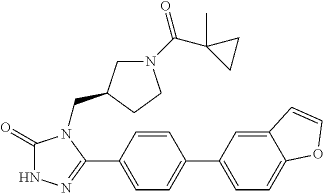 [SMILES: CC1(C(=O)N2CC[C@@H](Cn3c(-c4ccc(-c5ccc6occc6c5)cc4)n[nH]c3=O)C2)CC1]

To a reaction vial were added 5-[4-(1-benzofuran-5-yl)phenyl]-4-[(3R)-3-pyrrolidinylmethyl]-2,4-dihydro-3H-1,2,4-triazol-3-one hydrochloride (50 mg, 0.126 mmol), 1-methylcyclopropanecarboxylic acid (12.61 mg, 0.126 mmol), EDC (60.4 mg, 0.315 mmol), and HOBt (48.2 mg, 0.315 mmol), and the vial was purged with N₂. DMF (840 μl) and DIPEA (66.0 μl, 0.378 mmol) were added and the mixture was stirred at room temperature. After 1 h, LCMS analysis indicated conversion to the desired product. The mixture was purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (33 mg, 60%) as a white solid. MS (ES)⁺ m/e 443.1 [M+H]⁺.

### Example 98

### 5-[4-(1-benzofuran-5-yl)phenyl]-4-{[(3R)-1-(trifluoroacetyl)-3-pyrrolidinyl]methyl}-2,4-dihydro-3H-1,2,4-triazol-3-one

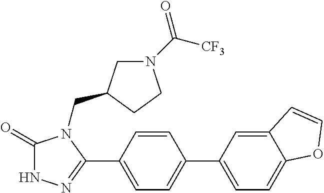 [SMILES: O=C(N1CC[C@@H](Cn2c(-c3ccc(-c4ccc5occc5c4)cc3)n[nH]c2=O)C1)C(F)(F)F]

To a reaction vial were added 5-[4-(1-benzofuran-5-yl)phenyl]-4-[(3R)-3-pyrrolidinylmethyl]-2,4-dihydro-3H-1,2,4-triazol-3-one hydrochloride (41 mg, 0.103 mmol) and NMP (800 ul), and the vial was purged with N₂. Trifluoroacetyl chloride was bubbled into the solution for 5 seconds. The solution was stirred at room temperature for 30 min. and LCMS analysis indicated desired product formation. The mixture was purified by reverse phase HPLC (10-90% CH₃CN/water+0.1% TFA). The desired fractions were collected and added to a reparatory funnel containing EtOAc and saturated aq. NaHCO₃. The aqueous phase was extracted thrice with EtOAc and the combined organic layers were washed with brine, dried over Na₂SO₄, and concentrated to afford the title compound (16 mg, 37%) as an off-white solid. MS (ES)⁺ m/e 457.1 [M+H]⁺.

### Example 99

### 5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer A)

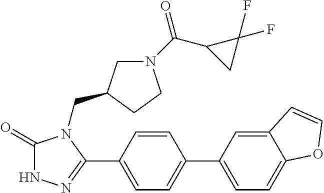 [SMILES: O=C(C1CC1(F)F)N1CC[C@@H](Cn2c(-c3ccc(-c4ccc5occc5c4)cc3)n[nH]c2=O)C1]

### a) 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one

The procedure described for the preparation of Example 97 was followed replacing 1-methylcyclopropanecarboxylic acid with racemic 2,2-difluorocyclopropanecarboxylic acid which provided the solid, 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one (700 mg, 34%) as a mixture of diasteromers. MS (ES)⁺ m/e 427.0, 428.9 [M+H]⁺. This material was further separated via chiral prep SFC (Chiralcel OD-H, 30×250 mm, 25% MeOH, 70 mL/min, UV-254 nm), to afford two white solids: 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one (Diastereomer A, Retention time: 6.3 min, 170 mg; 99.0% d.e.) and 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one (Diastereomer B, Retention time: 7.9 min, 203 mg; 99.2% d.e.)

### b) 5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer A)

To a microwave vial were added 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one (Diastereomer A, 56 mg, 0.131 mmol), 5-(4,4,5,5-tetramethyl-1,3,2-dioxaborolan-2-yl)-1-benzofuran (53 mg, 0.217 mmol), Cs₂CO₃ (155 mg, 0.476 mmol), and PdCl₂(dppf) (9 mg, 11 μmol), and the vial was purged with N₂. 1,4-Dioxane (1 mL) and water (0.5 mL) were added and the mixture was heated overnight on a hot plate at 100° C. LCMS analysis indicated desired product formation and consumption of the starting material. The mixture was filtered through a syringe filter and purified by reverse phase HPLC (10-90% CH₃CN/water+0.1% TFA). The desired fractions were collected and added to a reparatory funnel containing EtOAc and saturated aq. NaHCO₃. The aqueous phase was extracted thrice with EtOAc and the combined organic layers were washed with brine, dried over Na₂SO₄, and concentrated to afford the title compound (Diastereomer A, 37 mg, 61%) as a white solid. MS (ES)⁺ m/e 465.2 [M+H]⁺.

### Example 100

### 5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer B)

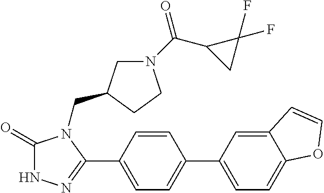 [SMILES: O=C(C1CC1(F)F)N1CC[C@@H](Cn2c(-c3ccc(-c4ccc5occc5c4)cc3)n[nH]c2=O)C1]

The procedure described for the preparation of Example 99(b) was followed replacing 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one (Diastereomer A) with 5-(4-bromophenyl)-4-({(3R)-1-[(2,2-difluorocyclopropyl)carbonyl]-3-pyrrolidinyl}methyl)-2,4-dihydro-3H-1,2,4-triazol-3-one (Diastereomer B), which provided the title compound as a white solid (25 mg, 47%). MS (ES)⁺ m/e 465.2 [M+H]⁺.

### Example 101

### N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylurea

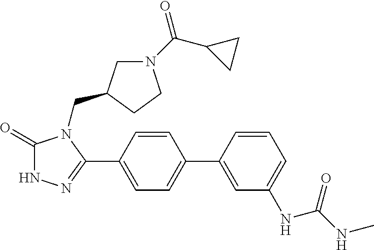 [SMILES: CNC(=O)Nc1cccc(-c2ccc(-c3n[nH]c(=O)n3C[C@@H]3CCN(C(=O)C4CC4)C3)cc2)c1]

### a) N-(3-bromophenyl)-N′-methylurea

To a microwave vial, (3-bromophenyl)amine 3-bromoaniline (850 μl, 7.81 mmol) was added under N₂ and dissolved in CHCl₃ (17 mL). Methyl isocyanate (500 mg, 8.76 mmol) was added and the solution was heated to 70° C. for 16 h. LCMS analysis showed desired product formation. The mixture was added to a separatory funnel containing CH₂Cl₂ and saturated NaHCO₃. The aqueous phase was extracted thrice with CH₂Cl₂ and the combined organic layers were washed with brine, dried over Na₂SO₄, and concentrated. This material was triturated with Et₂O:heptane (˜7 mL:7 mL) and the desired product was collected via vacuum filtration to afford the title compound (1.2 g, 67%) as a white solid. MS (ES)⁺ m/e 228.9, 230.8 [M+H]⁺.

### b) N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylurea

The procedure described for the preparation of Example 82 was followed replacing 5-bromo-1,3-benzoxazole with N-(3-bromophenyl)-N′-methylurea, which provided the title compound as a yellow solid (12 mg, 16%). MS (ES)⁺ m/e 461.3 [M+H]⁺.

### Example 102

### N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylurea

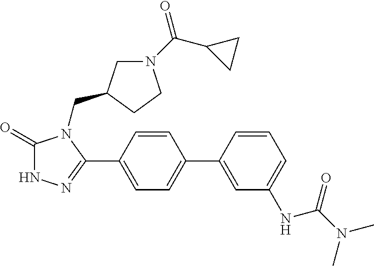 [SMILES: CN(C)C(=O)Nc1cccc(-c2ccc(-c3n[nH]c(=O)n3C[C@@H]3CCN(C(=O)C4CC4)C3)cc2)c1]

### a) N-(3-bromophenyl)-N,N-dimethylurea

To a microwave vial, 3-bromoaniline (300 μl, 2.755 mmol) and THF (2.5 mL) were added under N₂. Dimethylcarbamic chloride (279 μl, 3.031 mmol) and DIPEA (1925 μl, 11 mmol) were added and the solution was heated at 50° C. for 5 h. LCMS analysis showed desired product formation. The mixture was added to a separatory funnel containing EtOAc and saturated aq. NaHCO₃. The aqueous phase was extracted thrice with EtOAc and the combined organic layers were washed with brine, dried over Na₂SO₄, and concentrated. The residue was purified by silica gel chromatography (24 g column, 0-12% i-PrOH/EtOAc) and the desired fractions were combined to afford the title compound (504 mg, 75%) as a tan solid. MS (ES)⁺ m/e 243.0, 245.0 [M+H]⁺.

### b) N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylurea

The procedure described for the preparation of Example 82 was followed replacing 5-bromo-1,3-benzoxazole with N-(3-bromophenyl)-N,N-dimethylurea, which provided the title compound as a white solid (15 mg, 19%). MS (ES)⁺ m/e 475.1 [M+H]⁺.

### Example 103

### N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea

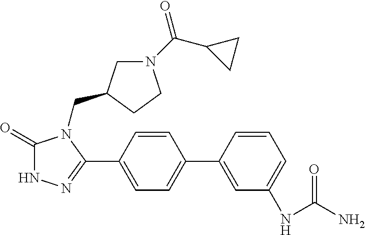 [SMILES: NC(=O)Nc1cccc(-c2ccc(-c3n[nH]c(=O)n3C[C@@H]3CCN(C(=O)C4CC4)C3)cc2)c1]

### a) N-(3-bromophenyl)urea

To a round bottom flask, KOCN (559 mg, 6889 μmol), water (7828 μl), and HOAc (783 μl) were added under N₂. 3-Bromoaniline (750 μl, 6889 μmol) was added slowly to the solution, and a precipitate began to form immediately. The mixture was stirred for 1 h at room temperature and LCMS analysis indicated desired product formation. The brown gummy precipitate was filtered off and dissolved in CH₂Cl₂. Heptane was added and a pinkish-white solid precipitated. This solid was collected and dried via vacuum filtration to afford the title compound. MS (ES)⁺ m/e 214.8, 217.0 [M+H]⁺.

### b) N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea

The procedure described for the preparation of Example 82 was followed replacing 5-bromo-1,3-benzoxazole with N-(3-bromophenyl)urea, which provided the title compound as a white solid (8 mg, 9%). MS (ES)⁺ m/e 447.2 [M+H]⁺.

### Example 104

### N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylsulfamide

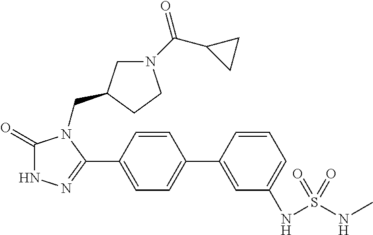 [SMILES: CNS(=O)(=O)Nc1cccc(-c2ccc(-c3n[nH]c(=O)n3C[C@@H]3CCN(C(=O)C4CC4)C3)cc2)c1]

### a) N-(3-bromophenyl)-N′-methylsulfamide

To a round bottom flask, methylsulfamic acid (1.62 g, 14.58 mmol) and PCl₅ (2.85 g, 13.69 mmol) were added under N₂ and dissolved in benzene (6 mL). This solution was heated to 50° C. for 5 min., then allowed to stir at room temperature for 30 min., and then heated to 70° C. for 1 h. LCMS analysis showed desired intermediate and the solution was concentrated. In a separate vial, 3-bromo aniline (2.5 g, 14.5 mmol) was dissolved in THF (50 mL) and then added to the concentrated material. The resulting solution was stirred for 30 min. at room temperature and LCMS analysis indicated desired product formation. This solution was concentrated and purified by reverse phase HPLC (10-90% CH₃CN/water+0.1% TFA). The desired fractions were combined and concentrated to afford the title compound as a tan solid. MS (ES)⁺ m/e 464.8, 266.8 [M+H]⁺.

### b) N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylsulfamide

The procedure described for the preparation of Example 82 was followed replacing 5-bromo-1,3-benzoxazole with N-(3-bromophenyl)-N′-methylsulfamide, which provided the title compound as a white solid (21 mg, 22%). MS (ES)⁺ m/e 497.1 [M+H]⁺.

### Example 105

### 5-(4-bromophenyl)-4-{[(3R)-1-(cyclopropylcarbonyl)-3-pyrrolidinyl]methyl}-2,4-dihydro-3H-1,2,4-triazol-3-one

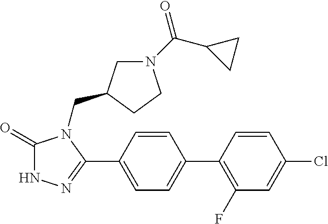 [SMILES: O=C(C1CC1)N1CC[C@@H](Cn2c(-c3ccc(-c4ccc(Cl)cc4F)cc3)n[nH]c2=O)C1]

To a microwave vial were added 5-(4-bromophenyl)-4-{[(3R)-1-(cyclopropylcarbonyl)-3-pyrrolidinyl]methyl}-2,4-dihydro-3H-1,2,4-triazol-3-one (60 mg, 0.153 mmol), (4-chloro-2-fluorophenyl)boronic acid (40.1 mg, 0.230 mmol), Cs₂CO₃ (150.5 mg, 0.460 mmol), and PdCl₂(dppf) (10.0 mg, 12.25 μmol), and the vial was purged with N₂. 1,4-Dioxane (1.0 mL) and water (0.5 mL) were added and the mixture was heated overnight on a hot plate at 100° C. LCMS analysis indicated desired product formation and consumption of the starting material. The mixture was filtered through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted thrice with EtOAc and the combined organic layers were washed with brine, dried over Na₂SO₄, and concentrated to afford the title compound (39 mg, 58%) as a tan solid. MS (ES)⁺ m/e 441.0, 443.0 [M+H]⁺.

### Example 106

### 5-[4-(1-benzofuran-5-yl)phenyl]-4-{[(3R)-1-(cyclopropylcarbonyl)-3-pyrrolidinyl]methyl}-2,4-dihydro-3H-1,2,4-triazole-3-thione

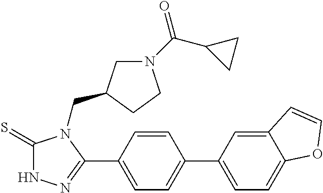 [SMILES: O=C(C1CC1)N1CC[C@@H](Cn2c(-c3ccc(-c4ccc5occc5c4)cc3)n[nH]c2=S)C1]

### a) 1,1-dimethylethyl(3S)-3-{[({2-[(4-bromophenyl)carbonyl]hydrazino}carbonothioyl)amino]methyl}-1-pyrrolidinecarboxylate

1,1-Dimethylethyl(3S)-3-(aminomethyl)-1-pyrrolidinecarboxylate (0.500 g, 2.497 mmol) and 1,1′-thiocarbonyldiimidazole (0.445 g, 2.497 mmol) were combined in dry CH₂Cl₂ (5 mL) and stirred at ambient temperature overnight. The reaction mixture was concentrated to dryness and the solvent replaced with dry THF (7 mL). The reaction mixture was treated with 4-bromobenzohydrazide (0.537 g, 2.497 mmol) at reflux for 2 h with stirring. The reaction mixture was concentrated to dryness and the residue was purified by flash chromatography (40-100% EtOAc/hexanes) to afford the title compound (0.86 g, 75%) as a white foam. LC-MS (ES) m/z 455.24, 457.18 [M−1]. LC-MS (ES⁺) m/z 457.24, 459.06 [M+H]⁺. ¹H NMR (400 MHz, DMSO-d₆) δ ppm 10.43 (s, 1H), 9.36 (s, 1H), 8.20-8.28 (m, 1H), 7.85 (d, J=8.52 Hz, 2H), 7.73 (d, J=8.52 Hz, 2H), 3.10-3.57 (m, 6H), 2.96 (dd, J=10.67, 6.56 Hz, 1H), 1.74-1.88 (m, 1H), 1.49-1.66 (m, 1H), 1.38 (s, 9H).

### b) 5-(4-bromophenyl)-4-{[(3R)-1-(cyclopropylcarbonyl)-3-pyrrolidinyl]methyl}-2,4-dihydro-3H-1,2,4-triazole-3-thione

1,1-Dimethylethyl(3S)-3-{[({2-[(4-bromophenyl)carbonyl]hydrazino}carbonothioyl)amino]methyl}-1-pyrrolidinecarboxylate (0.86 g, 1.880 mmol) was treated with 5% aq. K₂CO₃ (60 mL, 21.71 mmol) at reflux for 3 d, after which time analysis by LCMS indicated complete conversion to the deprotected and cyclized product. The reaction mixture was cooled to ambient temperature and treated dropwise with cyclopropanecarbonyl chloride (0.786 g, 7.52 mmol) and stirred for 4 h. Additional cyclopropanecarbonyl chloride (0.846 g, 8.09 mmol) was added to the reaction mixture, which was then stirred for 3 d. Analysis by LCMS still indicated ˜25% of the amine remained. 1 N aq. NaOH (20 mL, 20.00 mmol) was added to the reaction mixture followed by dropwise addition of cyclopropanecarbonyl chloride (0.79 g, 7.56 mmol). The mixture was stirred for 2 d at ambient temperature. The reaction mixture was extracted thrice with EtOAc. The aqueous layer was acidified to pH=1 with 1 N aq. HCl and extracted with EtOAc. The organic layers were combined and concentrated to dryness. The residue was purified via flash chromatography (40-100% EtOAc/hexanes) to afford the title compound (0.563 g, 74%) as a white foam. LC-MS (ES⁺) m/z 407.21, 409.07 [M+H]⁺. ¹H NMR (400 MHz, DMSO-d₆) δ ppm 14.07 (s, 1H), 7.73-7.82 (m, 2H), 7.64-7.72 (m, 2H), 4.13-4.21 (m, 2H), 2.84-3.56 (m, 4H), 2.35-2.58 (m, 1H), 1.32-1.89 (m, 3H), 0.58-0.69 (m, 4H).

### c) 5-[4-(1-benzofuran-5-yl)phenyl]-4-{[(3R)-1-(cyclopropylcarbonyl)-3-pyrrolidinyl]methyl}-2,4-dihydro-3H-1,2,4-triazole-3-thione

5-(4-Bromophenyl)-4-{[(3R)-1-(cyclopropylcarbonyl)-3-pyrrolidinyl]methyl}-2,4-dihydro-3H-1,2,4-triazole-3-thione (0.075 g, 0.184 mmol), 5-(4,4,5,5-tetramethyl-1,3,2-dioxaborolan-2-yl)-1-benzofuran (0.070 g, 0.287 mmol), K₃PO₄ (0.156 g, 0.737 mmol), and Pd(PPh₃)₄ (0.021 g, 0.018 mmol) were combined in EtOH (2 mL) and water (2 mL), purged with N₂, and irradiated in a microwave reactor for 1 h at 120° C., after which time analysis by LCMS indicated incomplete conversion. The reaction mixture was returned to the microwave reactor and irradiated an additional 3 h at 120° C., after which time analysis by LCMS indicated ˜50% conversion to product. The reaction mixture was filtered through Celite®, concentrated to dryness, taken into water, and extracted thrice with CH₂Cl₂. The organic phases were combined, dried over MgSO₄, filtered, and concentrated in vacuo. Purification of the residue by reverse phase HPLC (20-90% CH₃CN/water with 0.1% TFA) afforded the title product (0.0299 g, 36%) as a white foam. LC-MS (ES⁺) m/z 445.45 [M+H]⁺. ¹H NMR (400 MHz, DMSO-d₆) δ ppm 14.04 (s, 1H), 8.07 (d, J=2.15 Hz, 1H), 8.04 (s, 1H), 7.87-7.93 (m, 2H), 7.79-7.85 (m, 2H), 7.70-7.73 (m, 2H), 7.02-7.06 (m, 1H), 4.26 (dd, J=7.47, 3.81 Hz, 2H), 2.89-3.58 (m, 4H), 2.41-2.65 (m, 1H), 1.66-1.90 (m, 1H), 1.36-1.63 (m, 2H), 0.54-0.67 (m, 4H).

### Example 107

### 5-[2′-chloro-4′-(methyloxy)-4-biphenylyl]-4-{[(3R)-1-(cyclopropylcarbonyl)-3-pyrrolidinyl]methyl}-2,4-dihydro-3H-1,2,4-triazole-3-thione

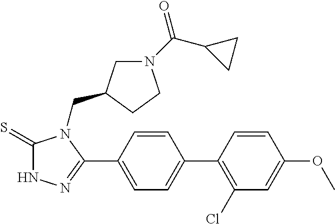 [SMILES: COc1ccc(-c2ccc(-c3n[nH]c(=S)n3C[C@@H]3CCN(C(=O)C4CC4)C3)cc2)c(Cl)c1]

The procedure described for the preparation of Example 106(c) was followed replacing 5-(4,4,5,5-tetramethyl-1,3,2-dioxaborolan-2-yl)-1-benzofuran with [2-chloro-4-(methyloxy)phenyl]boronic acid (2.0 equiv.), which gave the title product as a white foam (0.0256 g, 30%). LC-MS (ES⁺) m/z 469.42 [M+H]⁺. ¹H NMR (400 MHz, DMSO-d₆) δ ppm 14.05 (s, 1H), 7.80 (dd, J=8.17, 5.91 Hz, 2H), 7.59 (d, J=8.27 Hz, 2H), 7.41 (dd, J=8.54, 5.96 Hz, 1H), 7.19 (d, J=2.58 Hz, 1H), 7.05 (ddd, J=8.62, 2.50, 1.56 Hz, 1H), 4.17-4.29 (m, 2H), 3.84 (s, 3H), 2.91-3.60 (m, 4H), 2.43-2.64 (m, 1H), 1.66-1.89 (m, 1H), 1.34-1.64 (m, 2H), 0.57-0.70 (m, 4H).

### Example 108

### 4-{[(3R)-1-(cyclopropylcarbonyl)-3-pyrrolidinyl]methyl}-5-[4-(1H-indol-6-yl)phenyl]-2,4-dihydro-3H-1,2,4-triazole-3-thione

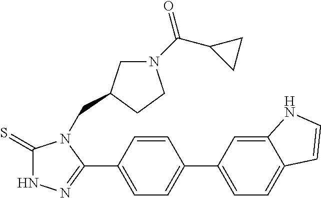 [SMILES: O=C(C1CC1)N1CC[C@@H](Cn2c(-c3ccc(-c4ccc5cc[nH]c5c4)cc3)n[nH]c2=S)C1]

The procedure described for the preparation of Example 106(c) was followed replacing 5-(4,4,5,5-tetramethyl-1,3,2-dioxaborolan-2-yl)-1-benzofuran with 1H-indol-6-ylboronic acid (2.4 equiv.), which gave the title product as a white solid (0.0328 g, 40%). LC-MS (ES⁻) m/z 442.09 [M−1]. LC-MS (ES⁺) m/z 443.98 [M+H]⁺. ¹H NMR (400 MHz, DMSO-d₆) δ ppm 14.03 (s, 1H), 11.26 (s, 1H), 7.36-7.91 (m, 8H), 6.47 (br. s., 1H), 4.26 (t, J=6.61 Hz, 2H), 2.91-3.57 (m, 4H), 2.43-2.66 (m, 1H), 1.67-1.90 (m, 1H), 1.38-1.64 (m, 2H), 0.55-0.69 (m, 4H).

### Example 109

### 4-{[(3R)-1-(cyclopropylcarbonyl)-3-pyrrolidinyl]methyl}-5-(4′-fluoro-4-biphenylyl)-2,4-dihydro-3H-1,2,4-triazole-3-thione

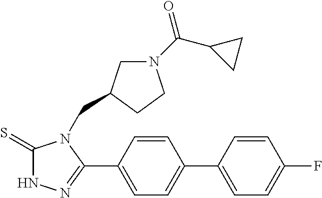 [SMILES: O=C(C1CC1)N1CC[C@@H](Cn2c(-c3ccc(-c4ccc(F)cc4)cc3)n[nH]c2=S)C1]

The procedure described for the preparation of Example 106(c) was followed replacing 5-(4,4,5,5-tetramethyl-1,3,2-dioxaborolan-2-yl)-1-benzofuran with (4-fluorophenyl)boronic acid (2.7 equiv.), which gave the title product as a white foam (0.0245 g, 32%). LC-MS (ES⁻) m/z 421.70 [M−1]. LC-MS (ES⁺) m/z 423.40 [M+H]⁺. ¹H NMR (400 MHz, DMSO-d₆) δ ppm 14.05 (s, 1H), 7.77-7.89 (m, 6H), 7.34 (m, 2H), 4.24 (dd, J=7.47, 3.28 Hz, 2H), 2.87-3.57 (m, 4H), 2.39-2.64 (m, 1H), 1.66-1.89 (m, 1H), 1.34-1.63 (m, 2H), 0.54-0.66 (m, 4H).

### Example 110

### 4-{[(3R)-1-(cyclopropylcarbonyl)-3-pyrrolidinyl]methyl}-5-(3,4′-difluoro-4-biphenylyl)-2,4-dihydro-3H-1,2,4-triazol-3-one

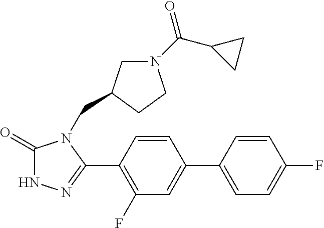 [SMILES: O=C(C1CC1)N1CC[C@@H](Cn2c(-c3ccc(-c4ccc(F)cc4)cc3F)n[nH]c2=O)C1]

The procedure described for Example 44 was used, replacing 2,4-difluorophenylboronic acid with 4-fluorophenylboronic acid (1.17 equiv.), to afford the title compound (49 mg, 46%). MS (ES)⁺ m/e 425.0 [M+H]⁺.

### Example 111

### 4′-(4-{[(3R)-1-(cyclopropylcarbonyl)-3-pyrrolidinyl]methyl}-5-oxo-4,5-dihydro-1H-1,2,4-triazol-3-yl)-3,3′-difluoro-4-biphenylcarbonitrile

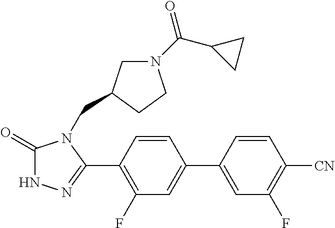 [SMILES: N#Cc1ccc(-c2ccc(-c3n[nH]c(=O)n3C[C@@H]3CCN(C(=O)C4CC4)C3)c(F)c2)cc1F]

The procedure described for Example 44 was used, replacing 2,4-difluorophenylboronic acid with 4-cyano-3-fluorophenylboronic acid (1.117 equiv.), to afford the title compound (42 mg, 38%). MS (ES)⁺ m/e 450.1 [M+H]⁺.

### Example 112

### 4-{[(3R)-1-(cyclopropylcarbonyl)-3-pyrrolidinyl]methyl}-5-[2′,3-difluoro-4′-(methyloxy)-4-biphenylyl]-2,4-dihydro-3H-1,2,4-triazol-3-one

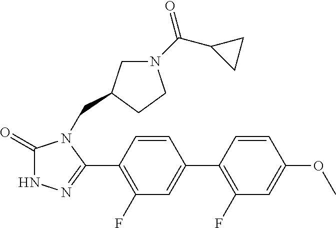 [SMILES: COc1ccc(-c2ccc(-c3n[nH]c(=O)n3C[C@@H]3CCN(C(=O)C4CC4)C3)c(F)c2)c(F)c1]

5-(4-Bromo-2-fluorophenyl)-4-{[(3R)-1-(cyclopropylcarbonyl)-3-pyrrolidinyl]methyl}-2,4-dihydro-3H-1,2,4-triazol-3-one (115 mg, 0.281 mmol), 2-fluoro-4-methoxyphenylboronic acid (55 mg, 0.324 mmol), PdCl₂(dppf) (20 mg, 0.024 mmol), 1,4-dioxane (2 mL), and 2 M aq. K₂CO₃ (1 mL) were placed in a 5 mL microwave vial. The vial was sealed and heated to 110° C. After 2 h, LCMS displayed the reaction had progressed to 50% completion. Added to the vial was 2-fluoro-4-methoxyphenylboronic acid (20 mg, 0.42 eq). The vial was capped and the reaction was stirred at 110° C. for 72 h. LCMS indicated that the reaction was complete with no starting materials present. The reaction mixture was cooled to room temperature, neutralized with 6 N aq. HCl and diluted with 20 mL EtOAc. The reaction mixture was washed with water. The organic layer was separated, dried over Na₂SO₄, filtered and concentrated to dryness. The crude material was taken up in 2 mL DMSO and purified by reverse phase HPLC, eluting with 10-70% CH₃CN/H₂O (0.1% NH₄OH). The desired fractions were collected and concentrated to dryness. The solid was triturated with hexane (3×) and dried to afford the title compound (20 mg, 15%). MS (ES)⁺ m/e 455.0 [M+H]⁺.

### Example 113

### 4-{[(3R)-1-(cyclopropylcarbonyl)-3-pyrrolidinyl]methyl}-5-(2′-fluoro-4-biphenylyl)-2,4-dihydro-3H-1,2,4-triazol-3-one

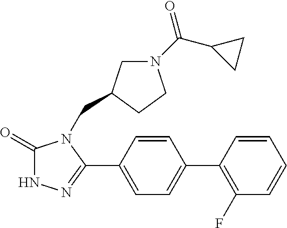 [SMILES: O=C(C1CC1)N1CC[C@@H](Cn2c(-c3ccc(-c4ccccc4F)cc3)n[nH]c2=O)C1]

5-(4-Bromophenyl)-4-{[(3R)-1-(cyclopropylcarbonyl)-3-pyrrolidinyl]methyl}-2,4-dihydro-3H-1,2,4-triazol-3-one (100 mg, 0.256 mmol), 2-fluorophenylboronic acid (40 mg, 0.286 mmol), PdCl₂(dppf)-CH₂Cl₂ adduct (20 mg, 0.025 mmol), 1,4-dioxane (2 mL), and 2 M aq. K₂CO₃ (1 mL) were placed in a 5 mL microwave vial. The vial was sealed and heated to 110° C. After 1 h, LCMS revealed that the reaction was complete. The reaction mixture was cooled to room temperature, neutralized with 6 N aq. HCl and diluted with 20 mL EtOAc. The mixture was washed with water. The organic layer was separated, dried over Na₂SO₄, filtered, and concentrated to dryness. The crude material was taken up in 2 mL DMSO and purified by reverse phase HPLC, eluting with 10-70% CH₃CN/H₂O (0.1% NH₄OH). The desired fractions were collected and concentrated to dryness to afford the title compound (65 mg, 62%). MS (ES)⁺ m/e 407.2 [M+H]⁺.

### Example 114

### 4-{[(3R)-1-(cyclopropylcarbonyl)-3-pyrrolidinyl]methyl}-5-(2′,3-difluoro-4-biphenylyl)-2,4-dihydro-3H-1,2,4-triazol-3-one

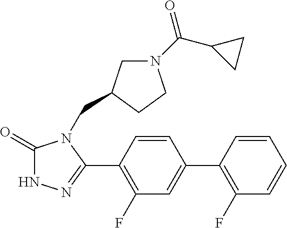 [SMILES: O=C(C1CC1)N1CC[C@@H](Cn2c(-c3ccc(-c4ccccc4F)cc3F)n[nH]c2=O)C1]

The procedure described for Example 44 was used, replacing 2,4-difluorophenylboronic acid with 2-fluorophenylboronic acid (1.125 equiv.), to afford the title compound (31 mg, 46%). MS (ES)⁺ m/e 425.0 [M+H]⁺.

### Example 115

### 4-{[(3R)-1-(cyclopropylcarbonyl)-3-pyrrolidinyl]methyl}-5-[2-fluoro-4-(7-quinolinyl)phenyl]-2,4-dihydro-3H-1,2,4-triazol-3-one

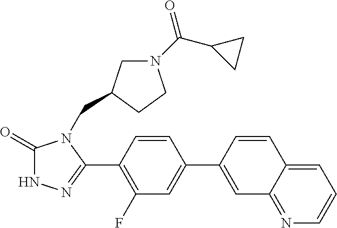 [SMILES: O=C(C1CC1)N1CC[C@@H](Cn2c(-c3ccc(-c4ccc5cccnc5c4)cc3F)n[nH]c2=O)C1]

5-(4-Bromo-2-fluorophenyl)-4-{[(3R)-1-(cyclopropylcarbonyl)-3-pyrrolidinyl]methyl}-2,4-dihydro-3H-1,2,4-triazol-3-one (100 mg, 0.244 mmol), 7-quinolineboronic acid pinacol ester (65 mg, 0.255 mmol), PdCl₂(dppf) (20 mg, 0.024 mmol), 1,4-dioxane (2 mL), and 2 M aq. K₂CO₃ (1 mL) were placed in a 5 mL microwave vial. The vial was sealed and heated to 110° C. After 1 h, LCMS revealed that the reaction was complete. The reaction mixture was cooled to room temperature, neutralized with 6 N aq. HCl, and diluted with 20 mL EtOAc. The reaction mixture was washed with water. The organic layer was separated, dried over Na₂SO₄, filtered, and concentrated to dryness. The crude material was taken up in 2 mL DMSO and purified by reverse phase HPLC, eluting with 10-70% CH₃CN/H₂O (0.1% NH₄OH). The desired fractions were collected and concentrated to dryness. The recovered material was purified by reverse phase HPLC, eluting with 5-35% CH₃CN/H₂O (0.1% TFA). The desired fractions were collected and concentrated to dryness in a 4 mL vial. To neutralize the TFA salt, added to the vial was DCM (2 mL) and MP-Carbonate (200 mg, 2.8 mmol/g). The solution was lightly agitated for 2 h at room temperature, then filtered and concentrated to dryness to afford the title compound (53 mg, 47%). MS (ES)⁺ m/e 458.2 [M+H]⁺.

### Example 116

### 4-{[(3R)-1-(cyclopropylcarbonyl)-3-pyrrolidinyl]methyl}-5-[2-fluoro-4-(3-quinolinyl)phenyl]-2,4-dihydro-3H-1,2,4-triazol-3-one

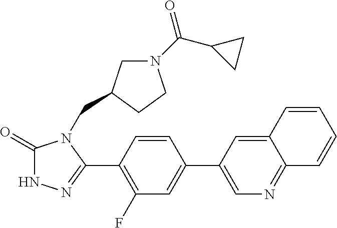 [SMILES: O=C(C1CC1)N1CC[C@@H](Cn2c(-c3ccc(-c4cnc5ccccc5c4)cc3F)n[nH]c2=O)C1]

5-(4-Bromo-2-fluorophenyl)-4-{[(3R)-1-(cyclopropylcarbonyl)-3-pyrrolidinyl]methyl}-2,4-dihydro-3H-1,2,4-triazol-3-one (100 mg, 0.244 mmol), 3-quinolineboronic acid (45 mg, 0.260 mmol), PdCl₂(dppf) (20 mg, 0.024 mmol), 1,4-dioxane (2 mL), and 2 M aq. K₂CO₃ (1 mL) were placed in a 5 mL microwave vial. The vial was sealed and heated to 110° C. After 1 h, LCMS revealed that the reaction was complete. The reaction mixture was cooled to room temperature, neutralized with 6 N aq. HCl, and diluted with 20 mL EtOAc. The reaction mixture was washed with water. The organic layer was separated, dried over Na₂SO₄, filtered, and concentrated to dryness. The crude material was taken up in 2 mL DMSO and purified by reverse phase HPLC, eluting with 10-70% CH₃CN/H₂O (0.1% NH₄OH). The desired fractions were collected and concentrated to dryness. The recovered material was purified by reverse phase HPLC, eluting with 2-35% CH₃CN/H₂O (no modifiers) to afford the title compound (38 mg, 34%). MS (ES)⁺ m/e 458.2 [M+H]⁺.

### Example 117

### 4′-(4-{[(3R)-1-(cyclopropylcarbonyl)-3-pyrrolidinyl]methyl}-5-oxo-4,5-dihydro-1H-1,2,4-triazol-3-yl)-2,3′-difluoro-4-biphenylcarbonitrile

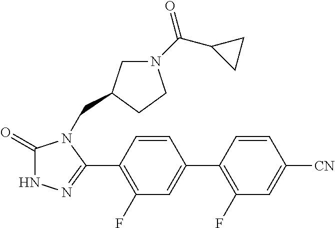 [SMILES: N#Cc1ccc(-c2ccc(-c3n[nH]c(=O)n3C[C@@H]3CCN(C(=O)C4CC4)C3)c(F)c2)c(F)c1]

5-(4-Bromo-2-fluorophenyl)-4-{[(3R)-1-(cyclopropylcarbonyl)-3-pyrrolidinyl]methyl}-2,4-dihydro-3H-1,2,4-triazol-3-one (123 mg, 0.301 mmol), potassium acetate (120 mg, 1.223 mmol), bis(pinacolato)diboron (85 mg, 0.335 mmol), PdCl₂(dppf)-CH₂Cl₂ adduct (25 mg, 0.031 mmol), and 1,4-dioxane (2 mL) were placed in a 5 mL microwave vial. The vial was sealed and heated to 110° C. After 2 h, LCMS analysis indicated that the boronate ester (and boronic acid) intermediate had completely formed. 4-Bromo-3-fluorobenzonitrile (65 mg, 0.325 mmol) and 2 M aq. K₂CO₃ (1 mL) were added. The vial was sealed and returned to stirring at 110° C. After 1 h, LCMS revealed that the reaction was complete. The reaction mixture was cooled to room temperature, neutralized with 6 N aq. HCl, and diluted with 20 mL EtOAc. The reaction mixture was washed with water. The organic layer was separated, dried over Na₂SO₄, filtered, and concentrated to dryness. The crude material was taken up in 2 mL DMSO and purified by reverse phase HPLC, eluting with 10-70% CH₃CN/H₂O (0.1% NH₄OH). The desired fractions were collected and concentrated to dryness to afford the title compound (61 mg, 45%). MS (ES)⁺ m/e 450.0 [M+H]⁺.

### Example 118

### 4-{[(3R)-1-(cyclopropylcarbonyl)-3-pyrrolidinyl]methyl}-5-[4-(2-hydroxy-3-quinolinyl)phenyl]-2,4-dihydro-3H-1,2,4-triazol-3-one

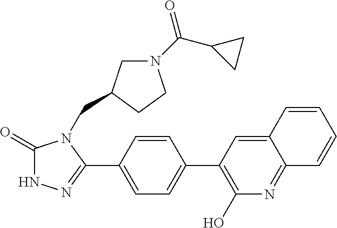 [SMILES: O=C(C1CC1)N1CC[C@@H](Cn2c(-c3ccc(-c4cc5ccccc5nc4O)cc3)n[nH]c2=O)C1]

To a microwave vial were added 5-(4-bromophenyl)-4-{[(3R)-1-(cyclopropylcarbonyl)-3-pyrrolidinyl]methyl}-2,4-dihydro-3H-1,2,4-triazol-3-one (63 mg, 0.161 mmol), [2-fluoro-3-quinolinyl]boronic acid (48 mg, 0.251 mmol), Cs₂CO₃ (201 mg, 0.614 mmol), palladium(II) acetate (13 mg, 0.058 mmol), and S-phos (23 mg, 0.056 mmol), and the vial was purged with N₂. 1,4-Dioxane (1.0 mL) and water (0.5 mL) were added and the mixture was heated at 100° C. for 16 h. LCMS analysis indicated consumption of the starting material and formation of a new major peak (MS (ES)⁺ m/e 456.1 [M+H]⁺). The mixture was passed through a syringe filter and purified by reverse phase HPLC (10-90% CH₃CN/water+0.1% TFA). The desired fractions were collected and added to a separatory funnel containing EtOAc and saturated aq. NaHCO₃. The aqueous phase was extracted with EtOAc (3×) and the combined organic layers were washed with brine, dried over Na₂SO₄, and concentrated to afford the title compound (15 mg, 21%) as a white solid. MS (ES)⁺ m/e 456.1 [M+H]⁺.

### Example 119

### 4-{[(3R)-1-(cyclopropylcarbonyl)-3-pyrrolidinyl]methyl}-5-[4′-(methyloxy)-4-biphenylyl]-2,4-dihydro-3H-1,2,4-triazol-3-one-d₂

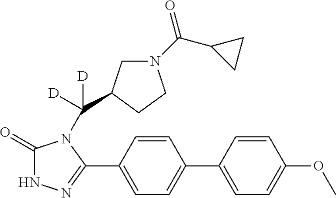 [SMILES: [2H]C([2H])([C@@H]1CCN(C(=O)C2CC2)C1)n1c(-c2ccc(-c3ccc(OC)cc3)cc2)n[nH]c1=O]

### a) (3S)-1-(phenylmethyl)-3-pyrrolidinyl methanesulfonate

A solution of (3S)-1-(phenylmethyl)-3-pyrrolidinol (10 g, 56.4 mmol) in CH₂Cl₂ (200 mL) and DIPEA (15.77 mL, 90 mmol) was cooled to 0° C. and treated with mesyl chloride (5.28 mL, 67.7 mmol) in a drop-wise fashion. The reaction was stirred and allowed to warm to room temperature under nitrogen gas. After 1 h, the solution was transferred to a separatory funnel and washed with saturated NaHCO₃ and brine. The organic phase was dried over Na₂SO₄, filtered and concentrated to dryness in vacuo to afford the title compound (13 g, 90% yield) which used without further purification. MS (ES)⁺ m/e 256.1 [M+H]⁺.

### b) (3R)-1-(phenylmethyl)-3-pyrrolidinecarbonitrile

A solution of (3S)-1-(phenylmethyl)-3-pyrrolidinyl methanesulfonate (13 g, 50.9 mmol) in DMF (100 mL) was treated with sodium cyanide (7.49 g, 153 mmol) in several portions. The solution was stirred under nitrogen gas at 100° C. After stirring for 18 h, the mixture was allowed to cool to room temperature, at which point the mixture was filtered through Celite®. The resulting brown filtrate was diluted with EtOAc (500 mL) and washed three times with brine. The organic phase was concentrated down to a residue. Purification of the residue by silica gel chromatography (0-100% EtOAc/hexanes gradient, 35 min) afforded the title compound (2.45 g, 26%) as a yellow tinted oil. MS (ES)⁺ m/e 187.0 [M+H]⁺.

### c) {[(3R)-1-(phenylmethyl)-3-pyrrolidinyl]methyl}amine-d₂

A solution of lithium aluminum deuteride (2.231 g, 53.2 mmol) in Et₂O (100 mL) was cooled to 0° C. in an ice water bath and was treated with a solution of (3R)-1-(phenylmethyl)-3-pyrrolidinecarbonitrile (2.2 g, 11.81 mmol) in diethyl ether (100 mL) in a drop-wise fashion. The mixture was stirred at 0° C. under nitrogen for 15 min. at which point it was removed from the ice bath and allowed to warm to room temperature. The reaction was quenched with water (1.95 mL), 15% NaOH in water (1.95 mL), and water 5.95 mL, sequentially, with ˜5 min. of stirring in between each addition. After the final addition, the mixture was stirred for 1 h. The resulting precipitate was filtered off and the filtrate was further diluted with Et₂O (100 mL). The organic phase was separated and the aqueous layer was extracted twice more with Et₂O. The combined Et₂O layer was washed with brine, dried over Na₂SO₄, filtered, and concentrated to dryness in vacuo to afford the title compound as a clear oil (2.3 g, quantitative yield). MS (ES)⁺ m/e 193.0 [M+H]⁺.

### d) 2-[(4-bromophenyl)carbonyl]-N-{[(3R)-1-(phenylmethyl)-3-pyrrolidinyl]methyl}hydrazinecarboxamide-d₂

A solution of {[(3R)-1-(phenylmethyl)-3-pyrrolidinyl]methyl}amine-d₂ (2.3 g, 11.96 mmol) was dissolved in CH₂Cl₂ (50 mL) was treated with carbonyldiimidazole (1.93 g, 11.96 mmol). The solution was stirred at room temperature under a nitrogen atmosphere. After 45 minutes the reaction mixture was concentrated to dryness in vacuo. The resulting yellow residue was taken up in THF (50 mL) and treated with 4-bromobenzohydrazide (2.57 g, 11.96 mmol) and DIPEA (3.13 mL, 17.94 mmol). The solution was stirred at 50° C. under nitrogen. After stirring for 18 h the solution was allowed to cool to room temperature at which point it was concentrated to dryness in vacuo. The residue was taken up in CH₂Cl₂ (100 mL) and washed with water and brine. The organic phase was then dried over Na₂SO₄, filtered and concentrated to dryness in vacuo. Purification of the residue by silica gel chromatography (0-10% MeOH/CH₂Cl₂ gradient, 60 min) afforded the title compound (3.02 g, 56%) as an off-white amorphous solid. MS (ES)⁺ m/e 433.1/434.8 [M+H]⁺.

### e) 5-(4-bromophenyl)-4-{[(3R)-1-(phenylmethyl)-3-pyrrolidinyl]methyl}-2,4-dihydro-3H-1,2,4-triazol-3-one-d₂

A suspension of 2-[(4-bromophenyl)carbonyl]-N-{[(3R)-1-(phenylmethyl)-3-pyrrolidinyl]methyl}hydrazinecarboxamide-d₂ (1 g, 2.308 mmol) in water (75 mL) was treated with potassium carbonate (1.595 g, 11.54 mmol). With vigorous stirring, the reaction mixture was heated to reflux (oil bath was set at 110° C.). After 18 h, the reaction was not yet complete, so the heat of the oil bath was increased to 120° C. for an additional 6 h. The reaction mixture was then allowed to cool to room temperature. Addition of 1N HCl was used to adjust the pH to ˜4. The aqueous mixture was then extracted with CH₂Cl₂, dried over Na₂SO₄, filtered and concentrated to dryness in vacuo. Purification of the residue by silica gel chromatography (0-10% MeOH/CH₂Cl₂ gradient, 20 min) afforded the title compound (385 mg, 40%) as a yellow amorphous solid. MS (ES)⁺ m/e 415.0/417.0 [M+H]⁺.

### f) 5-[4′-(methyloxy)-4-biphenylyl]-4-{[(3R)-1-(phenylmethyl)-3-pyrrolidinyl]methyl}-2,4-dihydro-3H-1,2,4-triazol-3-one-d₂

A solution of 5-(4-bromophenyl)-4-{[(3R)-1-(phenylmethyl)-3-pyrrolidinyl]methyl}-2,4-dihydro-3H-1,2,4-triazol-3-one-d₂ (173 mg, 0.417 mmol) in 1,4-dioxane (2.5 mL) was treated with [4-(methyloxy)phenyl]boronic acid (69.6 mg, 0.458 mmol), PdCl₂(dppf) (17.01 mg, 0.021 mmol), and aq. K₂CO₃ (0.625 mL, 1.250 mmol). The vessel was purged with nitrogen gas, sealed, and irradiated in a Biotage Initiator® microwave reactor at 150° C. for 15 min. The reaction solution was then diluted with water (50 mL) and its pH was adjusted to ˜7 with 1N HCl. The aqueous mixture was extracted with CH₂Cl₂. The combined organic phases were treated with Silicycle Si-thiol (˜20 mg) for 30 min., dried over Na₂SO₄, filtered, and concentrated to dryness in vacuo. Purification of the residue by silica gel chromatography (3-10% MeOH/CH₂Cl₂) afforded the title compound (102 mg, 55%) as a brown amorphous solid. MS (ES)⁺ m/e 443.2 [M+H]⁺.

### g) 4-{[(3R)-1-(cyclopropylcarbonyl)-3-pyrrolidinyl]methyl}-5-[4′-(methyloxy)-4-biphenylyl]-2,4-dihydro-3H-1,2,4-triazol-3-one-d₂

A solution of 5-[4′-(methyloxy)-4-biphenylyl]-4-{[(3R)-1-(phenylmethyl)-3-pyrrolidinyl]methyl}-2,4-dihydro-3H-1,2,4-triazol-3-one-d₂ (100 mg, 0.226 mmol) in MeOH (7 mL) was treated with 20% Pd(OH)₂ on carbon and H₂ at 70° C. for 2 h in an H-Cube™ Hydrogenation Reactor. The solution was concentrated to dryness in vacuo. The resulting off-white intermediate was then dissolved in CH₂Cl₂ (2 mL) and treated with DIPEA (0.079 mL, 0.452 mmol) and cyclopropanecarbonyl chloride (26.0 mg, 0.249 mmol) at room temperature. The reaction was stirred for 15 min. and the solution was concentrated to dryness in vacuo. The residue was taken up in MeOH (1 mL) and stirred at 40° C. After 3 h, the solution was concentrated to dryness in vacuo. Purification of the residue by silica gel chromatography (0-10% MeOH/CH₂Cl₂) followed by reverse phase HPLC (25-55% CH₃CN/water+0.1% TFA) afforded the title compound (27 mg, 27%) as an off-white amorphous solid. MS (ES)⁺ m/e 421.2 [M+H]⁺.

### Biological Assays

### FAS Assay

FAS activity was measured through one of the two following assays.

### Assay #1:

Inhibition of FAS activity can be measured based on the detection of residual NADPH substrate after the FAS assay is quenched. This assay is run as a 10 μL endpoint assay in 384-well format, where the reaction contains 20 μM malonyl-CoA, 2 μM acetyl-CoA, 30 μM NADPH and 40 nM FAS in 50 mM sodium phosphate, pH 7.0. The assay is run by sequentially dispensing 5 μl of a malonyl-CoA solution, then enzyme solution (containing the acetyl-CoA, and NADPH) into a black, low volume assay plate (Greiner 784076) pre-dispensed with 100 nL compound solutions in DMSO. The reaction is incubated at ambient temperature for 60 minutes, then quenched with 5 μL of a developing solution composed of 90 μM resazurin, 0.3 IU/ml diaphorase in 50 mM sodium phosphate, pH 7.0. The developed reaction is read on a Molecular Devices Analyst or Acquest (or equivalent) plate reader using a 530 nm excitation wavelength filter, a 580 nm emission filter, and 561 nm dichroic filter. The test compounds are prepared in neat DMSO at a concentration of 10 mM. For inhibition curves, compounds are diluted using a three fold serial dilution and tested at 11 concentrations (e.g. 25 μM-0.42 nM). Curves are analysed using ActivityBase and XLfit, and results are expressed as pIC50 values.

### Assay #2:

Inhibition of FAS can also be quantified based on the detection of the CoA products with a thio-reactive coumarin dye. This assay is run as a 10 μL endpoint assay in 384-well format, where the reaction contains 20 μM malonyl-CoA, 20 μM acetyl-CoA, 40 μM NADPH and 2 nM FAS in 50 mM sodium phosphate, pH 7.0, and 0.04% Tween-20. The assay is run by adding 5 μL enzyme solution to a black, low volume assay plate (Greiner 784076) pre-dispensed with 100 nl compound solutions in DMSO. After 30 minutes, 5 μL substrate is added, and the reaction incubated at ambient temperature for an additional 60 minutes. The reaction is then quenched with 10 μL of 6M guanidine-HCl containing 50 μM CPM (7-diethylamino-3-(4′-maleimidylphenyl)-4-methylcoumarin (CPM; thio-reactive dye), and incubated for 30 minutes. The plate is read on an Envision (PerkinElmer) or equivalent plate reader using a 380 nm excitation wavelength filter, and a 486 nm emission filter. Data fitting and compound preparations are done as described above.

### Biological Data

Exemplified compounds of the present invention (Examples 1-119) were tested according to the above assays and were found to be inhibitors of FAS. The IC₅₀ values ranged from about 1 to about 2,000 nM; the IC₅₀ values of the preferred compounds ranged from about 1 to about 100 nM. The compounds described below were tested generally according to the assays described herein. The IC₅₀ for each compound was either reported in at least one experiment or the average of multiple experiments.

Example 107: 72 nM

Example 105: 50 nM

Example 103: 316 nM

Example 97: 4 nM

Example 92: 25 nM

Example 91: 16 nM

Example 79: 100 nM

Example 78: 398 nM

Example 71: 20 nM

Example 66: 79 nM

Example 58: 1995 nM

Example 49: 40 nM

Example 47: 16 nM

Example 42: 263 nM

Example 35: 79 nM

Example 21: 4 nM

Example 20: 40 nM

Example 4: 25 nM

Example 2: 158 nM

### Lipogenesis Assay

Cultured primary human pre-adipocytes (Zen-Bio, Cat# ASC062801) are plated at confluence (3×104 cells/well) in 96-well plates (Costar, Cat#3598) coated with 0.2% gelatin (Sigma, Cat# G-6650) in DMEM/F12 medium (InvitroGen Cat#11330-032) supplemented with 10% heat inactivated fetal bovine serum (InvitroGen, Cat#16000-044). The following day (day 1) the cell differentiation is induced by replacing the seeding medium with the differentiation medium composed of DMEM/F12 medium supplemented with 10% heat inactivated fetal bovine serum, 200 μM 3-isobutyl-1-methylxanthine (Sigma, Cat# I-5879), 20 nM dexamethasone (Sigma, Cat# D-8893), 20 nM GW1929 (Sigma, Cat# G5668) and 20 nM insulin (InvitroGen, Cat#03-0110SA). On day 7, differentiation medium is replaced by the re-feed medium made of DMEM/F12 supplemented with 10% heat inactivated serum and 20 nM insulin. The appropriate concentration of tested compounds and controls are added into this medium at that time. On day 12, the relative amount of cellular triglyceride is estimated by using a Trinder kit (Sigma, Cat# TR0100). Re-feed medium is aspirated and cells are washed with PBS (InvitroGen, Cat#14190-144) and the assay is performed according the kit manufacturer protocol. Briefly, reconstituted solutions A and B are mixed with 0.01% digitonin (Sigma, Cat# D-5628) prior to performing the assay and added onto the cells; plates are incubated at 37° C. for one hour. The absorbance is read at 540 nm. The data is first normalized using the following equation: 100*((UNK−Control 1)/(Control 2−Control 1)) where Control 1 is the Robust Mean of the 0% response control and Control 2 is the Robust Mean of the 100% response control. When multiple dilutions of compounds are tested, pXC50 are calculated from curves using the 4-parameter curve fitting with the following equation: y=(a−d)/(1+(s/c)̂1))+d and with IRLS (Iterative Re-weighted Least Squares) algorithms to weight outliers (Mosteller, F. & Tukey J. W. (1977) Data Analysis and Regression, pp 353-365, Addison-Wesley).

1. A compound of Formula (I): 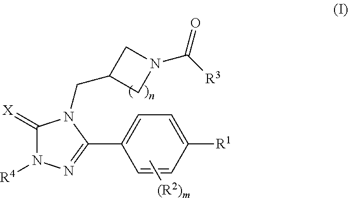
wherein:
R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;
when present each R² is independently selected from the group consisting of halogen, (C₁-C₆)alkyl, hydroxyl, and (C₁-C₄)alkoxy;
R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;
R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;
R⁵ is selected from the group consisting of hydrogen, (C₁-C₄)alkyl, (C₃-C₇)cycloalkyl, phenyl, and phenyl(C₁-C₃)alkyl-;
R⁶ is hydrogen, (C₁-C₄)alkyl, or (C₃-C₇)cycloalkyl;
or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁷ and R⁸ are each independently hydrogen, (C₁-C₄)alkyl, or (C₃-C₇)cycloalkyl;
or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶;
X is O or S;
m is 0-3; and
n is 1 or 2;
or pharmaceutically acceptable salts thereof.

2. A compound of Formula (I)(A): 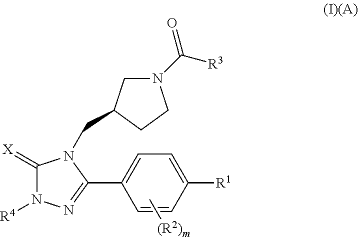
wherein:
R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;
when present each R² is independently selected from the group consisting of halogen, (C₁-C₆)alkyl, hydroxyl, and (C₁-C₄)alkoxy;
R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;
R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;
R⁵ is selected from the group consisting of hydrogen, (C₁-C₄)alkyl, (C₃-C₇)cycloalkyl, phenyl, and phenyl(C₁-C₃)alkyl-;
R⁶ is hydrogen, (C₁-C₄)alkyl, or (C₃-C₇)cycloalkyl;
or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁷ and R⁸ are each independently hydrogen, (C₁-C₄)alkyl, or (C₃-C₇)cycloalkyl;
or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶;
X is O or S; and
m is 0-3;
or pharmaceutically acceptable salts thereof.

3. A compound of Formula (I)(B): 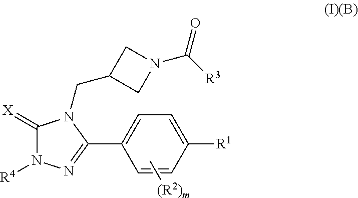
wherein:
R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;
when present each R² is independently selected from the group consisting of halogen, (C₁-C₆)alkyl, hydroxyl, and (C₁-C₄)alkoxy;
R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;
R⁴ is hydrogen, (C₁-C₆)alkyl, (C₃-C₇)cycloalkyl, —CO(C₁-C₄)alkyl, —CO(C₃-C₇)cycloalkyl, or —CONR⁵R⁶; wherein said (C₁-C₆)alkyl is optionally substituted by —CF₃, cyano, phenyl, —CONR⁵R⁶, hydroxyl, (C₁-C₄)alkoxy, or —NR⁵R⁶;
R⁵ is selected from the group consisting of hydrogen, (C₁-C₄)alkyl, (C₃-C₇)cycloalkyl, phenyl, and phenyl(C₁-C₃)alkyl-;
R⁶ is hydrogen, (C₁-C₄)alkyl, or (C₃-C₇)cycloalkyl;
or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁷ and R⁸ are each independently hydrogen, (C₁-C₄)alkyl, or (C₃-C₇)cycloalkyl;
or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶;
X is O or S; and
m is 0-3;
or pharmaceutically acceptable salts thereof.

4. The compound or salt according to claim 1, wherein R¹ is phenyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹.

5. The compound or salt according to claim 1, wherein R¹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹.

6. The compound or salt according to claim 1, wherein R¹ is benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹.

7. The compound or salt according to claim 1, wherein:
R² is fluoro, chloro, hydroxyl, methoxy, or methyl; and m is 1.

8. The compound or salt according to claim 1, wherein R³ is (C₁-C₄)alkyl, —CF₃, (C₃-C₆)cycloalkyl, methoxy, or dimethylamino, wherein said (C₃-C₆)cycloalkyl is optionally substituted 1 or 2 times independently by fluoro or methyl.

9. The compound or salt according to claim 1, wherein R³ is cyclopropyl.

10. The compound or salt according to claim 1, wherein R⁴ is hydrogen, (C₁-C₄)alkyl, —CO(C₁-C₄)alkyl, —CO(C₃-C₆)cycloalkyl, or —CONR⁷R⁸; wherein said (C₁-C₄)alkyl is optionally substituted by phenyl, —CONR⁷R⁸, hydroxyl, (C₁-C₄)alkoxy, or —NR⁷R⁸.

11. The compound according to claim 1 which is:
5-[4-(1-benzofuran-5-yl)-3-fluorophenyl]-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)-3-fluorophenyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-[3-fluoro-4-(1H-indol-5-yl)phenyl]-2,4-dihydro-3H-1,2,4-triazol-3-one;
4-{[(3R)-1-(cyclopropylcarbonyl)-3-pyrrolidinyl]methyl}-5-[3-fluoro-4-(1H-indol-6-yl)phenyl]-2,4-dihydro-3H-1,2,4-triazol-3-one;
5-[4-(1-benzofuran-5-yl)-2-fluorophenyl]-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)-2-fluorophenyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-[2-fluoro-4-(1H-indol-5-yl)phenyl]-2,4-dihydro-3H-1,2,4-triazol-3-one;
4-{[(3R)-1-(cyclopropylcarbonyl)-3-pyrrolidinyl]methyl}-5-[2-fluoro-4-(1H-indol-6-yl)phenyl]-2,4-dihydro-3H-1,2,4-triazol-3-one;
N′-[4′-(4-{[(3R)-1-(cyclopropylcarbonyl)-3-pyrrolidinyl]methyl}-5-oxo-4,5-dihydro-1H-1,2,4-triazol-3-yl)-3′-fluoro-3-biphenylyl]-N,N-dimethylsulfamide;
N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3′-fluoro-3-biphenylyl]-N,N-dimethylsulfamide;
4-{[(3R)-1-(cyclopropylcarbonyl)-3-pyrrolidinyl]methyl}-2-methyl-5-[2′,3,5′-trifluoro-4′-(methyloxy)-4-biphenylyl]-2,4-dihydro-3H-1,2,4-triazol-3-one;
5-[4-(1-benzofuran-5-yl)-2-methylphenyl]-4-{[(3R)-1-(cyclopropylcarbonyl)-3-pyrrolidinyl]methyl}-2,4-dihydro-3H-1,2,4-triazol-3-one;
N′-[4′-(4-{[(3R)-1-(cyclopropylcarbonyl)-3-pyrrolidinyl]methyl}-5-oxo-4,5-dihydro-1H-1,2,4-triazol-3-yl)-3′-methyl-3-biphenylyl]-N,N-dimethylsulfamide;
4-{[(3R)-1-(cyclopropylcarbonyl)-3-pyrrolidinyl]methyl}-5-[4-(1H-indol-6-yl)-2-methylphenyl]-2,4-dihydro-3H-1,2,4-triazol-3-one;
4-{[(3R)-1-(cyclopropylcarbonyl)-3-pyrrolidinyl]methyl}-5-(4′-fluoro-3-methyl-4-biphenylyl)-2,4-dihydro-3H-1,2,4-triazol-3-one;
4-{[(3R)-1-(cyclopropylcarbonyl)-3-pyrrolidinyl]methyl}-5-[4-(1H-indol-5-yl)-2-methylphenyl]-2,4-dihydro-3H-1,2,4-triazol-3-one;
5-[2-chloro-4-(1H-indol-6-yl)phenyl]-4-{[(3R)-1-(cyclopropylcarbonyl)-3-pyrrolidinyl]methyl}-2,4-dihydro-3H-1,2,4-triazol-3-one;
5-[2-chloro-4-(1H-indol-5-yl)phenyl]-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)-2-chlorophenyl]-4-{[(3R)-1-(cyclopropylcarbonyl)-3-pyrrolidinyl]methyl}-2,4-dihydro-3H-1,2,4-triazol-3-one;
5-(3-chloro-4′-fluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(1H-indol-6-yl)phenyl]-2,4-dihydro-3H-1,2,4-triazol-3-one;
5-[4-(1-benzofuran-5-yl)phenyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(1H-indazol-6-yl)phenyl]-2,4-dihydro-3H-1,2,4-triazol-3-one;
4-{[(3R)-1-(cyclopropylcarbonyl)-3-pyrrolidinyl]methyl}-5-[4-(1H-indazol-5-yl)phenyl]-2,4-dihydro-3H-1,2,4-triazol-3-one;
5-(4′-chloro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2,4-dihydro-3H-1,2,4-triazol-3-one;
5-[2′-chloro-4′-(methyloxy)-4-biphenylyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(1H-indol-5-yl)phenyl]-2,4-dihydro-3H-1,2,4-triazol-3-one;
5-[4-(1H-benzimidazol-5-yl)phenyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-(2′,4′-dichloro-4-biphenylyl)-2,4-dihydro-3H-1,2,4-triazol-3-one;
4-{[(3R)-1-(cyclopropylcarbonyl)-3-pyrrolidinyl]methyl}-5-[4-(1H-indol-6-yl)phenyl]-2-methyl-2,4-dihydro-3H-1,2,4-triazol-3-one;
5-[4-(1-benzofuran-5-yl)phenyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-[4-(1H-indol-5-yl)phenyl]-2-methyl-2,4-dihydro-3H-1,2,4-triazol-3-one;
4-{[(3R)-1-(cyclopropylcarbonyl)-3-pyrrolidinyl]methyl}-5-[4-(1H-indazol-6-yl)phenyl]-2-methyl-2,4-dihydro-3H-1,2,4-triazol-3-one;
5-[2′-chloro-4′-(methyloxy)-4-biphenylyl]-4-{[(3R)-1-(cyclopropylcarbonyl)-3-pyrrolidinyl]methyl}-2-methyl-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-methyl-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-(2-hydroxy-2-methylpropyl)-2,4-dihydro-3H-1,2,4-triazol-3-one;
2-acetyl-4-{[(3R)-1-(cyclopropylcarbonyl)-3-pyrrolidinyl]methyl}-5-(4′-fluoro-4-biphenylyl)-2,4-dihydro-3H-1,2,4-triazol-3-one;
2-(cyclopropylcarbonyl)-4-{[(3R)-1-(cyclopropylcarbonyl)-3-pyrrolidinyl]methyl}-5-(4′-fluoro-4-biphenylyl)-2,4-dihydro-3H-1,2,4-triazol-3-one;
4-{[(3R)-1-(cyclopropylcarbonyl)-3-pyrrolidinyl]methyl}-N-ethyl-3-(4′-fluoro-4-biphenylyl)-5-oxo-4,5-dihydro-1H-1,2,4-triazole-1-carboxamide;
4-{[(3R)-1-(cyclopropylcarbonyl)-3-pyrrolidinyl]methyl}-5-(4′-fluoro-4-biphenylyl)-2-[2-oxo-2-(1-pyrrolidinyl)ethyl]-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-(phenylmethyl)-2,4-dihydro-3H-1,2,4-triazol-3-one;
4-{[(3R)-1-(cyclopropylcarbonyl)-3-pyrrolidinyl]methyl}-5-(4′-fluoro-4-biphenylyl)-2-[2-(1-pyrrolidinyl)ethyl]-2,4-dihydro-3H-1,2,4-triazol-3-one;
4-{[(3R)-1-(cyclopropylcarbonyl)-3-pyrrolidinyl]methyl}-5-(2′,3,4′-trifluoro-4-biphenylyl)-2,4-dihydro-3H-1,2,4-triazol-3-one;
4-{[(3R)-1-(cyclopropylcarbonyl)-3-pyrrolidinyl]methyl}-5-[3-fluoro-4′-(methyloxy)-4-biphenylyl]-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-3′-fluoro-4-biphenylcarbonitrile;
5-(4′-chloro-3-fluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[3′-(methyloxy)-4-biphenylyl]-2,4-dihydro-3H-1,2,4-triazol-3-one;
N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylsulfamide;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]methanesulfonamide;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]acetamide;
4′-(4-{[(3R)-1-(cyclopropylcarbonyl)-3-pyrrolidinyl]methyl}-5-oxo-4,5-dihydro-1H-1,2,4-triazol-3-yl)-3-biphenylsulfonamide;
4′-(4-{[(3R)-1-(cyclopropylcarbonyl)-3-pyrrolidinyl]methyl}-5-oxo-4,5-dihydro-1H-1,2,4-triazol-3-yl)-3-biphenylcarboxamide;
4′-(4-{[(3R)-1-(cyclopropylcarbonyl)-3-pyrrolidinyl]methyl}-5-oxo-4,5-dihydro-1H-1,2,4-triazol-3-yl)-3-biphenylcarbonitrile;
4′-(4-{[(3R)-1-(cyclopropylcarbonyl)-3-pyrrolidinyl]methyl}-5-oxo-4,5-dihydro-1H-1,2,4-triazol-3-yl)-3-fluoro-4-biphenylcarbonitrile;
4-{[(3R)-1-(cyclopropylcarbonyl)-3-pyrrolidinyl]methyl}-5-[4-(7-methylimidazo[1,2-a]pyridin-6-yl)phenyl]-2,4-dihydro-3H-1,2,4-triazol-3-one;
5-(3′-acetyl-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(6-fluoro-3-pyridinyl)phenyl]-2,4-dihydro-3H-1,2,4-triazol-3-one;
4-{[(3R)-1-(cyclopropylcarbonyl)-3-pyrrolidinyl]methyl}-5-(4-imidazo[1,2-a]pyridin-6-ylphenyl)-2,4-dihydro-3H-1,2,4-triazol-3-one;
4-{[(3R)-1-(cyclopropylcarbonyl)-3-pyrrolidinyl]methyl}-5-[3′-(5-methyl-1,3,4-oxadiazol-2-yl)-4-biphenylyl]-2,4-dihydro-3H-1,2,4-triazol-3-one;
5-[3′,4′-bis(methyloxy)-4-biphenylyl]-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(2,3-dihydro-1-benzofuran-5-yl)phenyl]-2,4-dihydro-3H-1,2,4-triazol-3-one;
4-{[(3R)-1-(cyclopropylcarbonyl)-3-pyrrolidinyl]methyl}-5-{4′-[(trifluoromethyl)oxy]-4-biphenylyl}-2,4-dihydro-3H-1,2,4-triazol-3-one;
4-{[(3R)-1-(cyclopropylcarbonyl)-3-pyrrolidinyl]methyl}-5-[2′,5′-difluoro-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-[2′-fluoro-4′-(methyloxy)-4-biphenylyl]-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-4-biphenylcarbonitrile;
4′-(4-{[(3R)-1-(cyclopropylcarbonyl)-3-pyrrolidinyl]methyl}-5-oxo-4,5-dihydro-1H-1,2,4-triazol-3-yl)-4-biphenylcarboxamide;
N-butyl-N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea;
5-(2′-chloro-4′-fluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-[4-(3-quinolinyl)phenyl]-2,4-dihydro-3H-1,2,4-triazol-3-one;
4-{[(3R)-1-(cyclopropylcarbonyl)-3-pyrrolidinyl]methyl}-5-[4-(5-quinolinyl)phenyl]-2,4-dihydro-3H-1,2,4-triazol-3-one;
4-{[(3R)-1-(cyclopropylcarbonyl)-3-pyrrolidinyl]methyl}-5-[4-(6-quinolinyl)phenyl]-2,4-dihydro-3H-1,2,4-triazol-3-one;
4-{[(3R)-1-(cyclopropylcarbonyl)-3-pyrrolidinyl]methyl}-5-[4-(7-quinolinyl)phenyl]-2,4-dihydro-3H-1,2,4-triazol-3-one;
4-{[(3R)-1-(cyclopropylcarbonyl)-3-pyrrolidinyl]methyl}-5-[3′-hydroxy-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-[3′-methyl-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-[4-(8-methyl-5-quinolinyl)phenyl]-2,4-dihydro-3H-1,2,4-triazol-3-one;
5-(4′-chloro-2′,6′-difluoro-4-biphenylyl)-4-{[(3R)-1-(cyclopropylcarbonyl)-3-pyrrolidinyl]methyl}-2,4-dihydro-3H-1,2,4-triazol-3-one;
4-{[(3R)-1-(cyclopropylcarbonyl)-3-pyrrolidinyl]methyl}-5-(4-imidazo[1,2-a]pyridin-2-ylphenyl)-2,4-dihydro-3H-1,2,4-triazol-3-one;
4-{[(3R)-1-(cyclopropylcarbonyl)-3-pyrrolidinyl]methyl}-5-(4-imidazo[1,2-a]pyridin-7-ylphenyl)-2,4-dihydro-3H-1,2,4-triazol-3-one;
4-{[(3R)-1-(cyclopropylcarbonyl)-3-pyrrolidinyl]methyl}-5-[4-(8-isoquinolinyl)phenyl]-2,4-dihydro-3H-1,2,4-triazol-3-one;
5-[4-(3-amino-1H-indazol-6-yl)phenyl]-4-{[(3R)-1-(cyclopropylcarbonyl)-3-pyrrolidinyl]methyl}-2,4-dihydro-3H-1,2,4-triazol-3-one;
5-[4-(1,3-benzoxazol-5-yl)phenyl]-4-{[(3R)-1-(cyclopropylcarbonyl)-3-pyrrolidinyl]methyl}-2,4-dihydro-3H-1,2,4-triazol-3-one;
N′-[4′-(4-{[(3R)-1-(cyclopropylcarbonyl)-3-pyrrolidinyl]methyl}-1-methyl-5-oxo-4,5-dihydro-1H-1,2,4-triazol-3-yl)-3-biphenylyl]-N,N-dimethylsulfamide;
4′-(4-{[(3R)-1-(cyclopropylcarbonyl)-3-pyrrolidinyl]methyl}-1-methyl-5-oxo-4,5-dihydro-1H-1,2,4-triazol-3-yl)-3-fluoro-4-biphenylcarbonitrile;
4-{[(3R)-1-(cyclopropylcarbonyl)-3-pyrrolidinyl]methyl}-5-(4-imidazo[1,2-a]pyridin-6-ylphenyl)-2-methyl-2,4-dihydro-3H-1,2,4-triazol-3-one;
5-[4-(3-amino-1H-indazol-6-yl)phenyl]-4-{[(3R)-1-(cyclopropylcarbonyl)-3-pyrrolidinyl]methyl}-2-methyl-2,4-dihydro-3H-1,2,4-triazol-3-one;
5-[4-(4-{[(3R)-1-(cyclopropylcarbonyl)-3-pyrrolidinyl]methyl}-5-oxo-4,5-dihydro-1H-1,2,4-triazol-3-yl)phenyl]-3-methyl-1-benzofuran-2-carboxylic acid;
4-{[(3R)-1-(cyclopropylcarbonyl)-3-pyrrolidinyl]methyl}-5-[4-(3-methyl-1-benzofuran-5-yl)phenyl]-2,4-dihydro-3H-1,2,4-triazol-3-one;
5-[4-(4-{[(3R)-1-(cyclopropylcarbonyl)-3-pyrrolidinyl]methyl}-5-oxo-4,5-dihydro-1H-1,2,4-triazol-3-yl)phenyl]-1H-indole-3-carbonitrile;
5-[4-(1-benzofuran-5-yl)phenyl]-4-{[(3R)-1-(cyclopentylcarbonyl)-3-pyrrolidinyl]methyl}-2,4-dihydro-3H-1,2,4-triazol-3-one;
5-[4-(1-benzofuran-5-yl)phenyl]-4-{[(3R)-1-(2-methylpropanoyl)-3-pyrrolidinyl]methyl}-2,4-dihydro-3H-1,2,4-triazol-3-one;
(3R)-3-({3-[4-(1-benzofuran-5-yl)phenyl]-5-oxo-1,5-dihydro-4H-1,2,4-triazol-4-yl}methyl)-N,N-dimethyl-1-pyrrolidinecarboxamide;
5-[4-(1-benzofuran-5-yl)phenyl]-4-{[(3R)-1-propanoyl-3-pyrrolidinyl]methyl}-2,4-dihydro-3H-1,2,4-triazol-3-one;
methyl(3S)-3-({3-[4-(1-benzofuran-5-yl)phenyl]-5-oxo-1,5-dihydro-4H-1,2,4-triazol-4-yl}methyl)-1-pyrrolidinecarboxylate;
4-{[(3R)-1-acetyl-3-pyrrolidinyl]methyl}-5-[4-(1-benzofuran-5-yl)phenyl]-2,4-dihydro-3H-1,2,4-triazol-3-one;
5-[4-(1-benzofuran-5-yl)phenyl]-4-{[(3R)-1-(2,2-dimethylpropanoyl)-3-pyrrolidinyl]methyl}-2,4-dihydro-3H-1,2,4-triazol-3-one;
5-[4-(1-benzofuran-5-yl)phenyl]-4-({(3R)-1-[(1-methylcyclopropyl)carbonyl]-3-pyrrolidinyl}methyl)-2,4-dihydro-3H-1,2,4-triazol-3-one;
5-[4-(1-benzofuran-5-yl)phenyl]-4-{[(3R)-1-(trifluoroacetyl)-3-pyrrolidinyl]methyl}-2,4-dihydro-3H-1,2,4-triazol-3-one;
5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer A);
5-[4-(1-benzofuran-5-yl)phenyl]-4-({(3R)-1-[(2,2-difluorocyclopropyl)carbonyl]-3-pyrrolidinyl}methyl)-2,4-dihydro-3H-1,2,4-triazol-3-one (Diastereomer B);
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylurea;
N′-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N,N-dimethylurea;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]urea;
N-[4′-(4-{[(3R)-1-(cyclopropylcarbonyl)-3-pyrrolidinyl]methyl}-5-oxo-4,5-dihydro-1H-1,2,4-triazol-3-yl)-3-biphenylyl]-N′-methylsulfamide;
5-(4-bromophenyl)-4-{[(3R)-1-(cyclopropylcarbonyl)-3-pyrrolidinyl]methyl}-2,4-dihydro-3H-1,2,4-triazol-3-one;
5-[4-(1-benzofuran-5-yl)phenyl]-4-{[(3R)-1-(cyclopropylcarbonyl)-3-pyrrolidinyl]methyl}-2,4-dihydro-3H-1,2,4-triazole-3-thione;
5-[2′-chloro-4′-(methyloxy)-4-biphenylyl]-4-{[(3R)-1-(cyclopropylcarbonyl)-3-pyrrolidinyl]methyl}-2,4-dihydro-3H-1,2,4-triazole-3-thione;
4-{[(3R)-1-(cyclopropylcarbonyl)-3-pyrrolidinyl]methyl}-5-[4-(1H-indol-6-yl)phenyl]-2,4-dihydro-3H-1,2,4-triazole-3-thione;
4-{[(3R)-1-(cyclopropylcarbonyl)-3-pyrrolidinyl]methyl}-5-(4′-fluoro-4-biphenylyl)-2,4-dihydro-3H-1,2,4-triazole-3-thione;
4-{[(3R)-1-(cyclopropylcarbonyl)-3-pyrrolidinyl]methyl}-5-(3,4′-difluoro-4-biphenylyl)-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-3,3′-difluoro-4-biphenylcarbonitrile;
4-{[(3R)-1-(cyclopropylcarbonyl)-3-pyrrolidinyl]methyl}-5-[2′,3-difluoro-4′-(methyloxy)-4-biphenylyl]-2,4-dihydro-3H-1,2,4-triazol-3-one;
4-{[(3R)-1-(cyclopropylcarbonyl)-3-pyrrolidinyl]methyl}-5-(2′-fluoro-4-biphenylyl)-2,4-dihydro-3H-1,2,4-triazol-3-one;
4-{[(3R)-1-(cyclopropylcarbonyl)-3-pyrrolidinyl]methyl}-5-(2′,3-difluoro-4-biphenylyl)-2,4-dihydro-3H-1,2,4-triazol-3-one;
4-{[(3R)-1-(cyclopropylcarbonyl)-3-pyrrolidinyl]methyl}-5-[2-fluoro-4-(7-quinolinyl)phenyl]-2,4-dihydro-3H-1,2,4-triazol-3-one;
4-{[(3R)-1-(cyclopropylcarbonyl)-3-pyrrolidinyl]methyl}-5-[2-fluoro-4-(3-quinolinyl)phenyl]-2,4-dihydro-3H-1,2,4-triazol-3-one;
4′-(4-{[(3R)-1-(cyclopropylcarbonyl)-3-pyrrolidinyl]methyl}-5-oxo-4,5-dihydro-1H-1,2,4-triazol-3-yl)-2,3′-difluoro-4-biphenylcarbonitrile;
4-{[(3R)-1-(cyclopropylcarbonyl)-3-pyrrolidinyl]methyl}-5-[4-(2-hydroxy-3-quinolinyl)phenyl]-2,4-dihydro-3H-1,2,4-triazol-3-one; or
4-{[(3R)-1-(cyclopropylcarbonyl)-3-pyrrolidinyl]methyl}-5-[4′-(methyloxy)-4-biphenylyl]-2,4-dihydro-3H-1,2,4-triazol-3-one-d₂;
or pharmaceutically acceptable salts thereof.

12. A pharmaceutical composition comprising the compound according to claim 1 and a pharmaceutically acceptable carrier.

13. A method of treating cancer which comprises administering to a human in need thereof an effective amount of the compound according to claim 1.

14. The method of claim 13 wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, renal, ovarian, pancreatic, prostate, sarcoma, osteosarcoma, and giant cell tumor of bone and thyroid.
